SEQUENCE LISTING (109 sequences)

SEQ ID NO: 1 - protein, 332 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 2 - protein, 63 aa, Drosophila sp.
WKTNKSESQYTSLEYDFRVTCDLNYYGSGCAKFCRPRDDSFGHSTCSETGEIICLTGWQGDYC

SEQ ID NO: 3 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 4 - protein, 63 aa, Mus sp.
WSQDLHSSGRTDLRYSYRFVCDEHYYGEGCSVFCRPRDDAFGHFTCGDRGEKMCDPGWKGQYC

SEQ ID NO: 5 - protein, 63 aa, Rattus sp.
WSQDLHSSGRTDLRYSYRFVCDEHYYGEGCSVFCRPRDDAFGHFTCGERGEKMCDPGWKGQYC

SEQ ID NO: 6 - protein, 63 aa, Mus sp.
WRTDEQNDTLTRLSYSYRVICSDNYYGESCSRLCKKRDDHFGHYECQPDGSLSCLPGWTGKYC

SEQ ID NO: 7 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 8 - protein, 63 aa, Rattus sp.
WQTLKQNTGIAHFEYQIRVTCDDHYYGFGCNKFCRPRDDFFGHYACDQNGNKTCMEGWMGPEC

SEQ ID NO: 9 - protein, 63 aa, Mus sp.
WQTLKQNTGIAHFEYQIRVTCDDHYYGFGCNKFCRPRDDFFGHYACDQNGNKTCMEGWMGPDC

SEQ ID NO: 10 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 11 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 12 - protein, 63 aa, Gallus sp.
WKTLQFNGPVANFEVQIRVKCDENYYSALCNKFCGPRDDFVGHYTCDQNGNKACMEGWMGEEC

SEQ ID NO: 13 - protein, 63 aa, Mus sp.
WKSLHFSGHVAHLELQIRVRCDENYYSATCNKFCRPRNDFFGHYTCDQYGNKACMDGWMGKEC

SEQ ID NO: 14 - protein, 63 aa, Homo sapiens; identical to SEQ ID NO: 13

SEQ ID NO: 15 - protein, 63 aa, Rattus sp.; identical to SEQ ID NO: 13

SEQ ID NO: 16 - protein, 63 aa, Homo sapiens; identical to SEQ ID NO: 13

SEQ ID NO: 17 - protein, 63 aa, Drosophila sp.
WKTLDHIGRNARITYRVRVQCAVTYYNTTCTTFCRPRDDQFGHYACGSEGQKLCLNGWQGVNC

SEQ ID NO: 18 - protein, 723 aa, Homo sapiens [sequence omitted]

SEQ ID NO: 19 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 20 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 21 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 22 - protein, 1238 aa, Homo sapiens [sequence omitted]

SEQ ID NO: 23 - protein, 1424 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTPGYPGIPGAAGLKGQKGAPAKGEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTLGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPESLFHQL

SEQ ID NO: 24 - protein, 1049 aa, Homo sapiens
MDWSFFRVVAVLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 25 - protein, 2649 aa, Homo sapiens
MHSSSYSYRSSDSVFSNTTSTRTSLDSNENLLLVHCGPTLINSCISFGSESFDGHRLEMLQQIANRVQRDSVICEDKLILAGNALQSDSKRLESGVQFQNEAEIAGYILECENLLRQHVIDVQILIDGKYYQADQLVQRVAKLRDEIMALRNECSSVYSKGRILTTEQTKLMISGITQSLNSGFAQTLHPSLTSGLTQSLTPSLTSSSMTSGLSSGMTSRLTPSVTPAYTPGFPSGLVPNFSSGVEPNSLQTLKLMQIRKPLLKSSLLDQNLTEEEINMKFVQDLLNWVDEMQVQLDRTEWGSDLPSVESHLENHKNVHRAIEEFESSLKEAKISEIQMTAPLKLTYAEKLHRLESQYAKLLNTSRNQERHLDTLHNFVSRATNELIWLNEKEEEEVAYDWSERNTNIARKKDYHAELMRELDQKEENIKSVQEIAEQLLLENHPARLTIEAYRAAMQTQWSWILQLCQCVEQHIKENTAYFEFFNDAKEATDYLRNLKDAIQRKYSCDRSSSIHKLEDLVQESMEEKEELLQYKSTIANLMGKAKTIIQLKPRNSDCPLKTSIPIKAICDYRQIEITIYKDDECVLANNSHRAKWKVISPTGNEAMVPSVCFTVPPPNKEAVDLANRIEQQYQNVLTLWHESHINMKSVVSWHYLINEIDRIRASNVASIKTMLPGEHQQVLSNLQSRFEDFLEDSQESQVFSGSDITQLEKEVNVCKQYYQELLKSAEREEQEESVYNLYISEVRNIRLRLENCEDRLIRQIRTPLERDDLHESVFRITEQEKLKKELERLKDDLGTITNKCEEFFSQAAASSSVPTLRSELNVVLQNMNQVYSMSSTYIDKLKTVNLVLKNTQAAEALVKLYETKLCEEEAVIADKNNIENLISTLKQWRSEVDEKRQVFHALEDELQKAKAISDEMFKTYKERDLDFDWHKEKADQLVERWQNVHVQIDNRLRDLEGIGKSLKYYRDTYHPLDDWIQQVETTQRKIQENQPENSKTLATQLNQQKMLVSEIEMKQSKMDECQKYAEQYSATVKDYELQTMTYRAMVDSQQKSPVKRRRMQSSADLIIQEFMDLRTRYTALVTLMTQYIKFAGDSLKRLEEEEIKRCKETSEHGAYSDLLQRQKATVLENSKLTGKISELERMVAELKKQKSRVEEELPKVREAAENELRKQQRNVEDISLQKIRAESEAKQYRRELETIVREKEAAERELERVRQLTIEAEAKRAAVEENLLNFRNQLEENTFTRRTLEDHLKRKDLSLNDLEQQKNKLMEELRRKRDNEEELLKLIKQMEKDLAFQKQVAEKQLKEKQKIELEARRKITEIQYTCRENALPVCPITQATSCRAVTGLQQEHDKQKAEELKQQVDELTAANRKAEQDMRELTYELNALQLEKTSSEEKARLLKDKLDETNNTLRCLKLELERKDQAEKGYSQQLRELGRQLNQTTGKAEEAMQEASDLKKIKRNYQLELESLNHEKGKLQREVDRITRAHAVAEKNIQHLNSQIHSFRDEKELERLQICQRKSDHLKEQFEKSHEQLLQNIKAEKENNDKIQRLNEELEKSNECAEMLKQKVEELTRQNNETKLMMQRIQAESENIVLEKQTIQQRCEALKIQADGFKDQLRSTNEHLHKQTKTEQDFQRKIKCLEEDLAKSQNLVSEFKQKCDQQNIIIQNTKKEVRNLNAELNASKEEKRRGEQKVQLQQAQVQELNNRLKKVQDELHLKTIEEQMTHRKMVLFQEESGKFKQSAEEFRKKMEKLMESKVITENDISGIRLDFVSLQQENSRAQENAKLCETNIKELERQLQQYREQMQQGQHMEANHYQKCQKLEDELIAQKREVENLKQKMDQQIKEHEHQLVLLQCEIQKKSTAKDCTFKPDFEMTVKECQHSGELSSRNTGHLHPTPRSPLLRWTQEPQPLEEKWQHRVVEQIPKEVQFQPPGAPLEKEKSQQCYSEYFSQTSTELQITFDETNPITRLSEIEKIRDQALNNSRPPVRYQDNACEMELVKVLTPLEIAKNKQYDMHTEVTTLKQEKNPVPSAEEWMLEGCRASGGLKKGDFLKKGLEPETFQNFDGDHACSVRDDEFKFQGLRHTVTARQLVEAKLLDMRTIEQLRLGLKTVEEVQKTLNKFLTKATSIAGLYLESTKEKISFASAAERIIIDKMVALAFLEAQAATGFIIDPISGQTYSVEDAVLKGVVDPEFRIRLLEAEKAAVGYSYSSKTLSVFQAMENRMLDRQKGKHILEAQIASGGVIDPVRGIRVPPEIALQQGLLNNAILQFLHEPSSNTRVFPNPNNKQALYYSELLRMCVFDVESQCFLFPFGERNISNLNVKKTHRISVVDTKTGSELTVYEAFQRNLIEKSIYLELSGQQYQWKEAMFFESYGHSSHMLTDTKTGLHFNINEAIEQGTIDKALVKKYQEGLITLTELADSLLSRLVPKKDLHSPVAGYWLTASGERISVLKASRRNLVDRITALRCLEAQVSTGGIIDPLTGKKYRVAEALHRGLVDEGFAQQLRQCELVITGIGHPITNKMMSVVEAVNANIINKEMGIRCLEFQYLTGGLIEPQVHSRLSIEEALQVGIIDVLIATKLKDQKSYVRNIICPQTKRKLTYKEALEKADFDFHTGLKLLEVSEPLMTGISSLYYSS

SEQ ID NO: 26 - protein, 15 aa, Homo sapiens
LNSKIAFKIVSQEPA

SEQ ID NO: 27 - protein, 15 aa, Homo sapiens
TPMFLLSRNTGEVRT

SEQ ID NO: 28 - protein, 256 aa, Homo sapiens
MAHRPPSPALASVLLALLLSGAARAAEIVGGHEAQPHSRPYMASLQMRGNPGSHFCGGTLIHPSFVLTAPHCLRDIPQRLVNVVLGAHNVRTQEPTQQHFSVAQVFLNNYDAENKLNDILLIQLSSPANLSASVTSVQLPQQDQPVPHGTQCLAMGWGRVGAHDPPAQVLQELNVTVVTFFCRPHNICTFVPRRKAGICFGDSGGPLICDGIIQGIDSFVIWGCATRLFPDFFTRVALYVDWIRSTLRRVEAKGRP

SEQ ID NO: 29 - protein, 1039 aa, Homo sapiens [sequence omitted]

SEQ ID NO: 30 - protein, 788 aa, Homo sapiens [sequence omitted]

SEQ ID NO: 31 - protein, 291 aa, Homo sapiens
MAALQEKKTCGQRMEEFQRYCWNPDTGQMLGRTLSRWVWISLYYVAFYVVMTGLFALCLYVLMQTVDPYTPDYQDQLRSPGVTLRPDVYGEKGLEIVYNVSDNRTWADLTQTLHAFLAGYSPAAQEDSINCTSEQYFFQESFRAPNHTKFSCKFTADMLQNCSGLADPNFGFEEGKPCFIIKMNRIVKFLPSNGSAPRVDCAFLDQPRELGQPLQVKYYPPNGTFSLHYFPYYGKKAQPHYSNPLVAAKLLNIPRNAEVAIVCKVMAEHVTFNNPHDPYEGKVEFKLKIEK

SEQ ID NO: 32 - DNA, 1545 bp, Homo sapiens
  modified_base (216): a, c, g, t, unknown or other
atggggctagaagcactggtgcccctggccatgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccagttgcggcgccgacttcgggacgtgttcagcctgcanctggcctggacgccggtggtcgtgctcaatgggctggcggccgtgcgcgaggcgctggtgacccacggcgaggacaccgccgaccgcccgcctgtgcccatcacccagatcctgggcttcgggccgcgttcccaaggggtgttcctggcgcgctatgggcccgcgtggcgcgagcagaggcgcttctccgtctccaccttgcgcaacttgggcctgggcaagaagtcgctggagcagtgggtgaccgaggaggcngcctgcctttgtgccgccttcgccaaccactccggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgctgaatgctgtccccgtcctcctgcatatcccngcgctggctggcaaggtcctacgcttccaaaaggctttcctgacccagctggatgagctgctaactgagcacaggatgacctgggacccagcccagcccccccgagacctgactgaggccttcctggcagagatggagaaggccaaggggaaccctgcgagcagcttcaatgatgagaacctgcgcatagtggtggctgacctgttctctgccgggatggtgaccacctcgaccacgctggcctggggcctcctgctcatgatcctacatccggatgtgcagcgccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaggtacagggcttcngcatccctaagggaacgacactcatcaccaacctgtcatcggtnctgaaggatgaggccgtctgggagaagcccttccgcttccaccccgaacacttcctggatgcccagggccactttgtgaagccggaggccttcctgcctttctcagcaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctcctagcccagaggctctaatgtac

SEQ ID NO: 33 - protein, 516 aa, Homo sapiens [sequence omitted]

SEQ ID NO: 34 - protein, 405 aa, Homo sapiens [sequence omitted]

SEQ ID NO: 35 - protein, 173 aa, Homo sapiens
MDVTIQHPWFKRTLGPFYPSRLFDQFFGEGLFEYDLLPFLSSTISPYYRQSLFRTVLDSGISEVRSDRDKFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 36 - protein, 508 aa, Homo sapiens
MWELVALLLLTLAYLFWPKRRCPGAKYPKSLLSLPLVGSLPFLPRHGHMHNNFFKLQKKYGPIYSVRMGTKTTVIVGHHQLAKEVLIKKGKDFSGRPQMATLDIASNNRKGIAFADSGAHWQLHRRLAMATFALFKDGDQKLEKIICQEISTLCDMLATHNGQSIDISFPVFVAVTNVISLICFNTSYKNGDPELNVIQNYNEGIIDNLSKDSLVDLVPWLKIFPNKTLEKLKSHVKIRNDLLNKILENYKEKFRSDSITNMLDTLMQAKMNSDNGNAGPDQDSELLSDNHILTTIGDIFGAGVETTTSVVKWTLAFLLHNPQVKKKLYEEIDQNVGFSRTPTISDRNRLLLLEATIREVLRLRPVAPMLIPHKANVDSSIGEFAVDKGTEVIINLWALHHNEKEWHQPDQFMPERFLNPAGTQLISPSVSYLPFGAGPRSCIGEILARQELFLIMAWLLQRFDLEVPDDGQLPSLEGIPKVVFLIDSFKVKIKVRQAWREAQAEGST

SEQ ID NO: 37 - protein, 860 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 38 - protein, 885 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMFLPSLGHRAPVSQAAKFDPSTKIYEISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 39 - protein, 355 aa, Homo sapiens
MAAPAFEPGRQSDLLVKLNRLMERCLRNSKCIDTESLCVVAGEKVWQIRVDLHLLNHDGNIIDAASIAAIVALCHFRRPDVSVQGDEVTLYTPEERDPVPLSIHHMPICVSFAFFQQGTYLLVDPNEREERVMDGLLVIAMNKHREICTIQSSGGIMLLKDQVLRCSKIAGVKVAEITELILKALENDQKVRKEGGKFGFAESIANQRITAFKMEKAPIDTSDVEEKAEEIIAEAEPPSEVVSTPVLWTPGTAQIGEGVENSWGDLEDSEKEDDEGGGDQAIILDGIKMDTGVEVSDIGSQDAPIILSDSEEEEMIILEPDKNPKKIRTQTTSAKQEKAPSKKPVKRRKKKRAAN

SEQ ID NO: 40 - protein, 372 aa, Homo sapiens
MAAPAFEPGRQSDLLVKLNRLMERCLRNSKCIDTESLCVVAGEKVWQIRVDLHLLNHDGNIIDAASIAAIVALCHFRRPDVSVQGDEVTLYTPEERDPVPLSIHHMPICVSFAFFQQGTYLLVDPNEREERVMDGLLVIAMNKHREICTIQSSGGIMLLKDQVLRCSKIAGVKVAEITELILKALENDQKVRKEGGKFGFAESIANQRITAFKMEKAPIDTSDVEEKAEEIIAEAEPPSEVVSTPVLWTPGTAQIGEGVENSWGDLEDSEKEDDEGGGDQAIILDGIKMDTGVEVSDIGSQELGFHHVGQTGLEFLTSDAPIILSDSEEEEMIILEPDKNPKKIRTQTTSAKQEKAPSKKPVKRRKKKRAAN

SEQ ID NO: 41 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 42 - protein, 712 aa, Homo sapiens
MGGEEKPIGAGEEKQKEGGKKKNKEGSGDGGRAELNPWPEYIYTRLEMYNILKAEHDSILAEKAEKDSKPIKVTLPDGKQVDAESWKTTPYQIACGISQGLADNTVIAKVNNVVWDLDRPLEEDCTLELLKFEDEEAQAVYWHSSAHIMGEGMERVYGGCLCYGPPIENGFYYDMYLEEGGVSSNDFSSLEALCKKIIKEKQAFERLEVKKETLLAMFKYNKFKCRILNEKVNTPTTTVYRCGPLIDLCRGPHVRHTGKIKALKIHKNSSTYWEGKADMETLQRIYGISFPDPKMLKEWEKFQEEAKNRDHRKIGRDQELYFFHELSPGSCFFLPKGVYIYNALIEFIRSEYRKRGFQEVVTPNIFNSRLWMTSGHWQHYSENMFSFEVEKELFALKPMNCPGHSLMFDHRPRSWRELPLRLADFGGLHRNELSGALTGLTRVRRFQQDDAHIFCAMEQIEDEIKGCLDFLRTVYSVFGFSFKLNLSTRPEKFLGDIEVWDQAEKQLENSLNEFGEKWELNSGDGAFYGPKIDIQIKDAIGRYHQCATIQLDFQLPIRFNLTYVSHDGEDKKRPVIVHRAILGSVERMIAILTENYGGKLAPFWLSPRQVMVVPVGPTCDEYAQNVRQQFHDAKFMADIDLDPGCTLNKKIRNAQLAQYNFILVVGEKEKITGTVNIRTRDNKVHGERTISETIERLQQLKEFRSKQAEEEF

SEQ ID NO: 43 - protein, 968 aa, Homo sapiens
MDSTLTASEIRQRFIDFFKRNEHTYVHSSATIPLDDPTLLFANAGMNQFKPIFLNTIDPSHPMAKLSRAANTQKCIRAGGKQNDLDDVGKDVYHHTFFEMLGSWSFGDYFKELACKMALELLTQEFGIPIERLYVTYFGGDEAAGLEADLECKQIWQNLGLDDTKILPGNMKDNFWEMGDTGPCGPCSEIHYDRIGGRDAAHLVNQDDPNVLEIWNLVFIQYNREADGILKPLPKKSIDTGMGLERLVSVLQNKMSNYDTDLFVPYFEAIQKGTGARPYTGKVGAEDADGIDMAYRVLADHARTITVALADGGRPDNTGRGYVLRRILRRAVRYAHEKLNASRGFFATLVDVVVQSLGDAFPELKKDPDMVKDIINEEEVQFLKTLSRGRRILDRKIQSLGDSKTIPGDTAWLLYDTYGFPVDLTGLIAEEKGLVVDMDGFEEERKLAQLKSQGKGAGGEDLIMLDIYAIEELRARGLEVTDDSPKYNYHLDSSGSYVFENTVATVMALRREKMFVEEVSTGQECGVVLDKTCFYAEQGGQIYDEGYLVKVDDSSEDKTEFTVKNAQVRGGYVLHIGTIYGDLKVGDQVWLFIDEPRRRPIMSNHTATHILNFALRSVLGEADQKGSLVAPDRLRFDFTAKGAMSTQQIKKAEEIANEMIEAAKAVYTQDCPLAAAKAIQGLRAVFDETYPDPVRVVSIGVPVSELLDDPSGPAGSLTSVEFCGGTHLRNSSHAGAFVIVTEEAIAKGIRRIVAVTGAEAQKALRKAESLKKCLSVMEAKVKAQTAPNKDVQREIADLGEALATAVIPQWQKDELRETLKSLKKVMDDLDRASKADVQKRVLEKTKQFIDSNPNQPLVILEMESGASAKALNEALKLFKMHSPQTSAMLFTVDNEAGKITCLCQVPQNAANRGLKASEWVQQVSGLMDGKGGGKDVSAQATGKNVGCLQEALQLATSFAQLRLGDVKN

SEQ ID NO: 44 - protein, 685 aa, Homo sapiens
MDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 45 - protein, 277 aa, Homo sapiens [sequence omitted]

SEQ ID NO: 46 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 47 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 48 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 49 - protein, 23 aa, Homo sapiens
GRTQDENPVVHFFKNIVTPRTPP

SEQ ID NO: 50 - protein, 22 aa, Homo sapiens
AVYVYIYFNTWTTCQFIAFPFK

SEQ ID NO: 51 - protein, 21 aa, Homo sapiens
SQRHGSKYLATASTMDHARHG

SEQ ID NO: 52 - protein, 20 aa, Homo sapiens
RDTGILDSIGRFFGGDRGAP

SEQ ID NO: 53 - protein, 20 aa, Homo sapiens
QKSHGRTQDENPVVHFFKNI

SEQ ID NO: 54 - protein, 14 aa, Homo sapiens
DENPVVHFFKNIVT

SEQ ID NO: 55 - protein, 15 aa, Homo sapiens
ENPVVHFFKNIVTPR

SEQ ID NO: 56 - protein, 13 aa, Homo sapiens
HFFKNIVTPRTPP

SEQ ID NO: 57 - protein, 14 aa, Homo sapiens
KGFKGVDAQGTLSK

SEQ ID NO: 58 - protein, 20 aa, Homo sapiens
VDAQGTLSKIFKLGGRDSRS

SEQ ID NO: 59 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 60 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 61 - protein, 979 aa, Homo sapiens
MRRPRRPGGLGGSGGLRLLLCLLLLSSRPGGCSAVSAHGCLFDRRLCSHLEVCIQDGLFGQCQVGVGQARPLLQVTSPVLQRLQGVLRQLMSQGLSWHDDLTQYVISQEMERIPRLRPPEPRPRDRSGLAPKRPGPAGELLLQDIPTGSAPAAQHRLPQPPVGKGGAGASSSLSPLQAELLPPLLEHLLLPPQPPHPSLSYEPALLQPYLFHQFGSRDGSRVSEGSPGMVSVGPLPKAEAPALFSRTASKGIFGDHPGHSYGDLPGPSPAQLFQDSGLLYLAQELPAPSRARVPRLPEQGSSSRAEDSPEGYEKEGLGDRGEKPASPAVQPDAALQRLAAVLAGYGVELRQLTPEQLSTLLTLLQLLPKGAGRNPGGVVNVGADIKKTMEGPVEGRDTAELPARTSPMPGHPTASPTSSEVQQVPSPVSSEPPKAARPPVTPVLLEKKSPLGQSQPTVAGQPSARPAAEEYGYIVTDQKPLSLAAGVKLLEILAEHVHMSSGSFINISVVGPALTFRIRHNEQNLSLADVTQQAGLVKSELEAQTGLQILQTGVGQREEAAAVLPQTAHSTSPMRSVLLTLVALAGVAGLLVALAVALCVRQHARQQDKERLAALGPEGAHGDTTFEYQDLCRQHMATKSLFNRAEGPPEPSRVSSVSSQFSDAAQASPSSHSSTPSWCEEPAQANMDISTGHMILAYMEDHLRNRDRLAKEWQALCAYQAEPNTCATAQGEGNIKKNRHPDFLPYDHARIKLKVESSPSRSDYINASPIIEHDPRMPAYIATQGPLSHTIADFWQMVWESGCTVIVMLTPLVEDGVKQCDRYWPDEGASLYHVYEVNLVSEHIWCEDFLVRSFYLKNVQTQETRTLTQFHFLSWPAEGTPASTRPLLDFRRKVNKCYRGRSCPIIVHCSDGAGRTGTYILIDMVLNRMAKGVKEIDIAATLEHVRDQRPGLVRSKDQFEFALTAVAEEVNAILKALPQ

SEQ ID NO: 62 - protein, 622 aa, Homo sapiens
MSMRSPISAQLALDGVGTMVNCTIKSEEKKEPCHEAPQGSATAAEPQPGDPARASQDSADPQAPAQGNFRGSWDCSSPEGNGSPEPKRPGASEAASGSQEKLDFNRNLKEVVPAIEKLLSSDWKERFLGRNSMEAKDVKGTQESLAEKELQLLVMIHQLSTLRDQLLTAHSEQKNMAAMLFEKQQQQMELARQQQEQIAKQQQQLIQQQHKINLLQQQIQQVNMPYVMIPAFPPSHQPLPVTPDSQLALPIQPIPCKPVEYPLQLLHSPPAPVVKRPGAMATHHPLQEPSQPLNLTAKPKAPELPNTSSSPSLKMSSCVPRPPSHGGPTRDLQSSPPSLPLGFLGEGDAVTKAIQDARQLLHSHSGALDGSPNTPFRKDLISLDSSPAKERLEDGCVHPLEEAMLSCDMDGSRHFPESRNSSHIKRPMNAFMVWAKDERRKILQAFPDMHNSSISKILGSRWKSMTNQEKQPYYEEQARLSRQHLEKYPDYKYKPRPKRTCIVEGKRLRVGEYKALMRTRRQDARQSYVIPPQAGQVQMSSSDVLYPRAAGMPLAQPLVEHYVPRSLDPNMPVIVNTCSLREEGEGTDDRHSVADGEMYRYSEDEDSEGEEKSDGELVVLTD

SEQ ID NO: 63 - protein, 483 aa, Homo sapiens [sequence omitted]

SEQ ID NO: 64 - protein, 471 aa, Homo sapiens
MSHHPSGLRAGFSSTSYRRTFGPPPSLSPGAFSYSSSSRFSSSRLLGSASPSSSVRLGSFRSPRAGAGALLRLPSERLDFSMAEALNQEFLATRSNEKQELQELNDRFANFIEKVRFLEQQNAALRGELSQARGQEPARADQLCQQELRELRRELELLGRERDRVQVERDGLAEDLAALKQRLEEETRKREDAEHNLVLFRKDVDDATLSRLELERKIESLMDEIEFLKKLHEEELRDLQVSVESQQVQQVEVEATVKPELTAALRDIRAQYESIAAKNLQEAEEWYKSKYADLSDAANRNHEALRQAKQEMNESRRQIQSLTCEVDGLRGTNEALLRQLRELEEQFALEAGGYQAGAARLEEELRQLKEEMARHLREYQELLNVKMALDIEIATYRKLLEGEESRISVPVHSFASLNIKTTVPEVEPPQDSHSRKTVLIKTIETRNGEQVVTESQKEQRSELDKSSAHSY

SEQ ID NO: 65 - protein, 306 aa, Homo sapiens
DCQAKSTPVIVSATTKKGLSSDLEGEKTTSLKWKSDEVDEQVACQEVKVSVAIEEDLEPENGILELETKSSKLVQNIIQTAVDQFVRTEETATEMLTSELQTQAHMIKADSQDAGQETEKEGEEPQASAQDETPITSAKEESESTAVGQAHSDISKDMSEASEKTMTVEVEGSTVNDQQLEEVVLPSEEEGGGAGTKSVPEDDGHALLAERIEKSLVEPKEDEKGDDVDDPENQNSALADTDASGGLTKESPDTNGPKQKEKEDAQEVELQEGKVHSESDKAITPQAQEELQKQERESAKSELTES

SEQ ID NO: 66 - protein, 520 aa, Homo sapiens
MHGGQGPLLLLLLLAVCLGGTQRNLRNQEERLLADLMQNYDPNLRPAERDSDVVNVSLKLTLTNLISLNEREEALTTNVWIEMQWCDYRLRWDPRDYEGLWVLRVPSTMVWRPDIVLENNVDGVFEVALYCNVLVSPDGCIYWLPPAIFRSACSISVTYFPFDWQNCSLIFQSQTYSTNEIDLQLSQEDGQTIEWIFIDPEAFTENGEWAIQHRPAKMLLDPAAPAQEAGHQKVVFYLLIQRKPLFYVINIIAPCVLISSVAILIHFLPAKAGGQKCTVAINVLLAQTVFLFLVAKKVPETSQAVPLISKYLTFLLVVTILIVVNAVVVLNVSLRSPHTHSMARGVRKVFLRLLPQLLRMHVRPLAPAAVQDTQSRLQNGSSGWSITTGEEVALCLPRSELLFQQWQRQGLVAAALEKLEKGPELGLSQFCGSLKQAAPAIQACVEACNLIACARHQQSHFDNGNEEWFLVGRVLDRVCFLAMLSLFICGTAGIFLMAHYNRVPALPFPGDPRPYLPSPD

SEQ ID NO: 67 - protein, 457 aa, Homo sapiens [sequence omitted]

SEQ ID NO: 68 - protein, 36 aa, Homo sapiens
TVGLQLIQLINVDEVNQIVTTNVRLKQQWVDYNLKW

SEQ ID NO: 69 - protein, 20 aa, Homo sapiens
QIVTTNVRLKQQWVDYNLKW

SEQ ID NO: 70 - protein, 7 aa, Homo sapiens
QWVDYNL

SEQ ID NO: 71 - protein, 18 aa, Homo sapiens
GGVKKIHIPSEKIWRPDL

SEQ ID NO: 72 - protein, 12 aa, Homo sapiens
AIVKFTKVLLQY

SEQ ID NO: 73 - protein, 20 aa, Homo sapiens
WTPPAIFKSYCEIIVTHFPF

SEQ ID NO: 74 - protein, 13 aa, Homo sapiens
MKLGTWTYDGSVV

SEQ ID NO: 75 - protein, 13 aa, Homo sapiens
MKLGIWTYDGSVV

SEQ ID NO: 76 - protein, 9 aa, Homo sapiens
WTYDGSVVA

SEQ ID NO: 77 - protein, 17 aa, Homo sapiens
SCCPDTPYLDITYHFVM

SEQ ID NO: 78 - protein, 18 aa, Homo sapiens
DTPYLDITYHFVMQRLPL

SEQ ID NO: 79 - protein, 21 aa, Homo sapiens
FIVNVIIPCLLFSFLTGLVFY

SEQ ID NO: 80 - protein, 13 aa, Homo sapiens
LLVIVELIPSTSS

SEQ ID NO: 81 - protein, 19 aa, Homo sapiens
STHVMPNWVRKVFIDTIPN

SEQ ID NO: 82 - protein, 18 aa, Homo sapiens
NWVRKVFIDTIPNIMFFS

SEQ ID NO: 83 - protein, 18 aa, Homo sapiens
IPNIMFFSTMKRPSREKQ

SEQ ID NO: 84 - protein, 16 aa, Homo sapiens
AAAEWKYVAMVMDHIL

SEQ ID NO: 85 - protein, 19 aa, Homo sapiens
IIGTLAVFAGRLIELNQQG

SEQ ID NO: 86 - protein, 20 aa, Homo sapiens
GQTIEWIFIDPEAFTENGEW

SEQ ID NO: 87 - protein, 20 aa, Homo sapiens
MAHYNRVPALPFPGDPRPYL

SEQ ID NO: 88 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 89 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 90 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 91 - protein, 475 aa, Homo sapiens [sequence omitted]

SEQ ID NO: 92 - protein, 764 aa, Homo sapiens [sequence omitted]

SEQ ID NO: 93 - protein, 933 aa, Homo sapiens [sequence omitted]

SEQ ID NO: 94 - protein, 43 aa, Artificial Sequence
  Description of Artificial Sequence Synthetic amino acid consensus sequence
CXXXXXXXXCXXXCXXXXXXXXXXXCXXXXXXXCXXXXXXXXC

SEQ ID NO: 95 - protein, 43 aa, Artificial Sequence; identical to SEQ ID NO: 94
  Description of Artificial Sequence Synthetic amino acid consensus sequence

SEQ ID NO: 96 - protein, 43 aa, Artificial Sequence
  Description of Artificial Sequence Synthetic amino acid consensus sequence
CXXXYYXXXCXXXCRPRBDXFGHXXCXXXGXXXCXXGWXGXXC

SEQ ID NO: 97 - protein, 175 aa, Artificial Sequence
  Description of Artificial Sequence Synthetic EGF-like domain
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 98 - protein, 864 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 21 aa, Mus musculus
MEVGWYRSPFSRVVHLYRNGK

SEQ ID NO: 100 - DNA, 26 bp, Artificial Sequence
  Description of Artificial Sequence Synthetic primer
caccatgggcagtcggtgcgcgctgg

SEQ ID NO: 101 - DNA, 45 bp, Artificial Sequence
  Description of Artificial Sequence Synthetic primer
gtctacgtttaaacttaacactcgtcaatccccagctcgcaggtg

SEQ ID NO: 102 - DNA, 999 bp, Artificial Sequence
  Description of Artificial Sequence Synthetic nucleotide construct
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgttaa

SEQ ID NO: 103 - protein, 332 aa, Artificial Sequence; identical to SEQ ID NO: 1
  Description of Artificial Sequence Synthetic protein construct

SEQ ID NO: 104 - protein, 13 aa, Homo sapiens
VHFFKNIVTPRTP

SEQ ID NO: 105 - protein, 7 aa, Homo sapiens
FKNIVTP

SEQ ID NO: 106 - protein, 6 aa, Artificial Sequence
  Description of Artificial Sequence Synthetic peptide
DKQTLL

SEQ ID NO: 107 - protein, 4 aa, Artificial Sequence
  Description of Artificial Sequence Synthetic peptide
KDEL

SEQ ID NO: 108 - protein, 6 aa, Artificial Sequence
  Description of Artificial Sequence Synthetic peptide
DEKKMP

SEQ ID NO: 109 - protein, 525 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRALQNTLLNKSF

(19) United States
(12) Patent Application Publication
(10) Pub. No.: US 2006/0204508 A1
(43) Pub. Date: Sep. 14, 2006

(54) Treatment of autoimmune diseases using an activator for the notch signalling pathway

(76) Inventors: Brian Robert Champion, Cambridge (GB); Silvia Ragno, Cambridge (GB); Lesley Lynn Young, Cambridge (GB)

Correspondence Address: FROMMER LAWRENCE & HAUG, 745 FIFTH AVENUE- 10TH FL., NEW YORK, NY, 10151, US

(21) Appl. No.: 11/188,417

(22) Filed: Jul. 25, 2005

Related U.S. Application Data
(63) Continuation-in-part of application No. PCT/GB04/00263, filed on Jan. 23, 2004.

(30) Foreign Application Priority Data
Jan. 23, 2003 (GB) 0301519.5
Jan. 23, 2003 (GB) 0301518.7
Jan. 23, 2003 (GB) 0301515.3
Jan. 23, 2003 (GB) 0301513.8
Jan. 23, 2003 (GB) 0301512.0
Jan. 23, 2003 (GB) 0301510.4
Jan. 23, 2003 (GB) 0301521.1
Jan. 23, 2003 (GB) 0301522.9
Jan. 23, 2003 (GB) 0301524.5
Jan. 23, 2003 (GB) 0301526.0
Jan. 23, 2003 (GB) 0301527.8
Jan. 23, 2003 (GB) 0301529.4
May 24, 2003 (GB) 0312062.3
Oct. 3, 2003 (GB) 0323130.5
Apr. 4, 2003 (WO) PCT/GB03/01525
Aug. 1, 2003 (WO) PCT/GB03/03285
Jan. 7, 2004 (WO) PCT/GB04/00046

(51) Int. Cl. A61K 39/00; A61K 35/14

(52) U.S. Cl. 424/184.1; 424/93.7

(57) ABSTRACT

A product is disclosed comprising i) a modulator of the Notch signalling pathway; and ii) an autoantigen or bystander antigen, or a polynucleotide coding for an autoantigen or bystander antigen; as a combined preparation for simultaneous, contemporaneous, separate or sequential use for modulation of immune response.

### CROSS REFERENCE TO RELATED APPLICATIONS

This application is a continuation-in-part of International Application No. PCT/GB2004/000263, filed on Jan. 23, 2004, published as WO 2004/064863 on Aug. 5, 2004, and claiming priority to GB Application Serial Nos. 0301519.5, 0301518.7, 0301515.3, 0301513.8, 0301512.0, 0301510.4, 0301521.1, 0301522.9, 0301524.5, 0301526.0, 0301527.8, and 0301529.4, all filed Jan. 23, 2003, 0312062.3, filed May 24, 2003 and 0323130.5, filed Oct. 3, 2003 and to International Application Nos. PCT/GB03/001525, filed Apr. 4, 2003, PCT/GB03/003285, filed Aug. 1, 2003 and PCT/GB04/00046, filed Jan. 7, 2004.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; Ser. No. 11/058,066, filed Feb. 14, 2005; Ser. No. 11/071,796, filed Mar. 3, 2005; Ser. No. 11/078,735, filed Mar. 10, 2005 and Ser. No. 11/103,077, filed Apr. 11, 2005. Reference is also made to the application filed on Jul. 11, 2005, entitled “Therapeutic Use of Modulators of Notch,” having attorney docket number 674525-2021.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the modulation of immune function.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins.

A description of the Notch signalling pathway and conditions affected by it may be found in our published PCT Applications WO 98/20142, WO 00/36089 and WO 01/35990. The text of each of PCT/GB97/03058 (WO 98/20142), PCT/GB99/04233 (WO 00/36089) and PCT/GB00/04391 (WO 01/35990) is hereby incorporated herein by reference (see also Hoyne G. F. et al. (1999) Int Arch Allergy Immunol 118:122-124; Hoyne et al. (2000) Immunology 100:281-288; Hoyne G. F. et al. (2000) Intl Immunol 12:177-185; Hoyne, G. et al. (2001) Immunological Reviews 182:215-227).

A description of the Notch signalling pathway and conditions affected by it may be found, for example, in our published PCT Applications as follows:

PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);

PCT/GB99/04233 (filed on 15 Dec. 1999 and published as WO 00/36089; claiming priority from GB 9827604.1 filed on 15 Dec. 1999);

PCT/GB00/04391 (filed on 17 Nov. 2000 and published as WO 0135990; claiming priority from GB 9927328.6 filed on 18 Nov. 1999);

PCT/GB01/03503 (filed on 3 Aug. 2001 and published as WO 02/12890; claiming priority from GB 0019242.7 filed on 4 Aug. 2000);

PCT/GB02/02438 (filed on 24 May 2002 and published as WO 02/096952; claiming priority from GB 0112818.0 filed on 25 May 2001);

PCT/GB02/03381 (filed on 25 Jul. 2002 and published as WO 03/012111; claiming priority from GB 0118155.1 filed on 25 Jul. 2001);

PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);

PCT/GB02/04390 (filed on 27 Sep. 2002 and published as WO 03/029293; claiming priority from GB 0123379.0 filed on 28 Sep. 2001);

PCT/GB02/05137 (filed on 13 Nov. 2002 and published as WO 03/041735; claiming priority from GB 0127267.3 filed on 14 Nov. 2001, PCT/GB02/03426 filed on 25 Jul. 2002, GB 0220849.4 filed on 7 Sep. 2002, GB 0220913.8 filed on 10 Sep. 2002 and PCT/GB02/004390 filed on 27 Sep. 2002);

PCT/GB02/05133 (filed on 13 Nov. 2002 and published as WO 03/042246; claiming priority from GB 0127271.5 filed on 14 Nov. 2001 and GB 0220913.8 filed on 10 Sep. 2002); PCT/GB2003/001525 (filed on 4 Apr. 2003), published as WO 03/087159; and PCT/GB2003/003285 filed on 1 Aug. 2003 (claiming priority from GB 0312062.3 and others).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735), PCT/GB02/05133 (WO 03/042246) and PCT/GB2003/001525 (WO 03/087159) and PCT/GB2003/003285, is hereby incorporated herein by reference

The present invention seeks to provide further methods of modulating the immune system particularly, but without limitation, in the prevention and/or treatment of autoimmune disease.

For example, according to one aspect of the present invention, it has suprisingly been found that Notch signalling provides a “bystander effect” or “bystander suppression effect” which may be used in a wide variety of ways to suppress unwanted immune responses in immune diseases and disorders. In particular, this “Notch bystander effect” may, for example, be used to provide targeted immune suppression at a disease locus with less of an undesirable general immunosuppressant effect on the whole body as compared, for example, to immunoppressant drugs or steroids, which are relatively indiscriminate in action.

The “Notch bystander effect” identified in one aspect of the present invention is particularly suited to treatment of autoimmune disease, but is not limited to such treatment, and may also be used to treat other immune related disorders. Use of the effect makes it possible to provide localised immune suppression in an autoimmune disease even where the primary autoantigen or autoantigens are uncertain or not fully characterised, so long as a relevant “bystander antigen” can be identified. Thus, when using this “bystander effect” it is not always necessary to identify key pathogenic autoantigens as targets for immune suppression (although this will be possible in some cases). Chosen antigens or antigenic determinants may simply need to be expressed in or delivered to the diseased tissue (or lymphatic tissue draining these sites).

### SUMMARY OF THE INVENTION

According to a first aspect of the invention there is provided a product comprising a modulator of the Notch signalling pathway and an autoantigen or bystander antigen or antigenic determinant thereof or a polynucleotide coding for an autoantigen or bystander antigen or antigenic determinant thereof as a combined preparation for simultaneous, contemporaneous, separate or sequential use for modulation of immune response.

According to a further aspect of the invention there is provided a method of modulating the immune system in a mammal comprising simultaneously, contemporaneously, separately or sequentially administering to a mammal in need thereof an effective amount of a modulator of the Notch signalling pathway and an effective amount of an autoantigen or bystander antigen or antigenic determinant thereof or a polynucleotide coding for an autoantigen or bystander antigen or antigenic determinant thereof.

According to a further aspect of the invention there is provided a combination of a modulator of the Notch signalling pathway and an autoantigen or bystander antigen or antigenic determinant thereof or a polynucleotide coding for an autoantigen or bystander antigen or antigenic determinant thereof; for simultaneous, contemporaneous, separate or sequential use in modulating the immune system.

According to a further aspect of the invention there is provided a modulator of the Notch signalling pathway for use in modulating the immune system in simultaneous, contemporaneous, separate or sequential combination with an autoantigen or bystander antigen or antigenic determinant thereof or a polynucleotide coding for an autoantigen or bystander antigen or antigenic determinant thereof.

According to a further aspect of the invention there is provided the use of a combination of a modulator of the Notch signalling pathway and an autoantigen or bystander antigen or antigenic determinant thereof or a polynucleotide coding for an autoantigen or bystander antigen or antigenic determinant thereof; in the manufacture of a medicament for modulation of immune response.

According to a further aspect of the invention there is provided the use of a modulator of the Notch signalling pathway in the manufacture of a medicament for modulation of immune response in simultaneous, contemporaneous, separate or sequential combination with an autoantigen or bystander antigen or antigenic determinant thereof or a polynucleotide coding for an autoantigen or bystander antigen or antigenic determinant thereof.

According to a further aspect of the invention there is provided a pharmaceutical kit comprising a modulator of the Notch signalling pathway and an autoantigen or bystander antigen or antigenic determinant thereof or a polynucleotide coding for an autoantigen or bystander antigen or antigenic determinant thereof.

Preferably, in any aspect of the present invention, the modulator of the Notch signalling pathway will be an activator of the Notch signalling pathway, and preferably a direct activator of a Notch receptor (“Notch receptor agonist”), such as a Notch ligand or fragment, derivative or variant thereof.

Preferably the methods, uses, products and compositions etc. of the present invention are for in vivo (rather than ex-vivo or in vitro) administration.

Preferably, for treatment of humans, the autoantigen or bystander antigen or antigenic determinant for use with the invention in any of its aspects will be a human autoantigen, bystander antigen or antigenic determinant thereof, or a polynucleotide coding for any of the foregoing.

Preferably the modulation of immune response comprises immunotherapy.

Preferably the modulation of immune response comprises reducing the immune response to the autoantigen or bystander antigen.

Preferably the modulation of immune response comprises modulation of T-cell activity, preferably peripheral T-cell activity.

According to one embodiment the modulation of immune response comprises treatment of an organ-specific autoimmune disease.

According to an alternative embodiment the modulation of immune response comprises treatment of a systemic autoimmune disease.

In one embodiment of the present invention the autoantigen or bystander antigen may be a Goodpasture's autoantigen or bystander antigen for treatment of Goodpasture's disease.

According to this aspect of the invention there is further provided a product comprising a modulator of the Notch signalling pathway and a Goodpasture's autoantigen or bystander antigen or antigenic determinant thereof or a polynucleotide coding for a Goodpasture's autoantigen or bystander antigen or antigenic determinant thereof as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Goodpasture's autoantigen or bystander antigen or antigenic determinant thereof or a polynucleotide coding for a Goodpasture's autoantigen or bystander antigen or antigenic determinant thereof.

According to this aspect of the invention there is further provided a combination of a modulator of the Notch signalling pathway and a Goodpasture's autoantigen or bystander antigen or antigenic determinant thereof or a polynucleotide coding for a Goodpasture's autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Goodpasture's autoantigen or bystander antigen or antigenic determinant thereof or a polynucleotide coding for a Goodpasture's autoantigen or bystander antigen or antigenic determinant thereof.

According to this aspect of the invention there is further provided the use of a combination of a modulator of the Notch signalling pathway and a Goodpasture's autoantigen or bystander antigen or antigenic determinant thereof or a polynucleotide coding for a Goodpasture's autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Goodpasture's autoantigen or bystander antigen or antigenic determinant thereof or a polynucleotide coding for a Goodpasture's autoantigen or bystander antigen or antigenic determinant thereof.

According to this aspect of the invention there is further provided a pharmaceutical kit comprising a modulator of the Notch signalling pathway and a Goodpasture's autoantigen or bystander antigen or antigenic determinant thereof or a polynucleotide coding for a Goodpasture's autoantigen or bystander antigen or antigenic determinant thereof.

In one embodiment the autoantigen or bystander antigen may be a renal autoantigen or renal bystander antigen for treatment of an autoimmune disease of the kidney.

According to this aspect of the invention there is further provided a product comprising a modulator of the Notch signalling pathway and a renal autoantigen or bystander antigen or antigenic determinant thereof, or a polynucleotide coding for a renal autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a renal autoantigen or bystander antigen or antigenic determinant thereof, or a polynucleotide coding for a renal autoantigen or bystander antigen or antigenic determinant thereof.

According to this aspect of the invention there is further provided a combination of a modulator of the Notch signalling pathway and a renal autoantigen or bystander antigen or antigenic determinant thereof, or a polynucleotide coding for a renal autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a renal autoantigen or bystander antigen or antigenic determinant thereof, or a polynucleotide coding for a renal autoantigen or bystander antigen or antigenic determinant thereof.

According to this aspect of the invention there is further provided the use of a combination of a modulator of the Notch signalling pathway and a renal autoantigen or bystander antigen or antigenic determinant thereof, or a polynucleotide coding for a renal autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a renal autoantigen or bystander antigen or antigenic determinant thereof, or a polynucleotide coding for a renal autoantigen or bystander antigen or antigenic determinant thereof.

According to this aspect of the invention there is further provided a pharmaceutical kit comprising a modulator of the Notch signalling pathway and a renal autoantigen or bystander antigen or antigenic determinant thereof, or a polynucleotide coding for a renal autoantigen or bystander antigen or antigenic determinant thereof.

In an alternative embodiment of the present invention the autoantigen or bystander antigen may be a Pemphigus autoantigen or bystander antigen for treatment of Pemphigus.

According to this aspect of the invention there is further provided a product comprising a modulator of the Notch signalling pathway and a Pemphigus autoantigen or bystander antigen or antigenic determinant thereof, or a polynucleotide coding for a Pemphigus autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Pemphigus autoantigen or bystander antigen or antigenic determinant thereof, or a polynucleotide coding for a Pemphigus autoantigen or bystander antigen or antigenic determinant thereof.

According to this aspect of the invention there is further provided a combination of a modulator of the Notch signalling pathway and a Pemphigus autoantigen or bystander antigen or antigenic determinant thereof, or a polynucleotide coding for a Pemphigus autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Pemphigus autoantigen or bystander antigen or antigenic determinant thereof, or a polynucleotide coding for a Pemphigus autoantigen or bystander antigen or antigenic determinant thereof.

According to this aspect of the invention there is further provided the use of a combination of a modulator of the Notch signalling pathway and a Pemphigus autoantigen or bystander antigen or antigenic determinant thereof, or a polynucleotide coding for a Pemphigus autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Pemphigus autoantigen or bystander antigen or antigenic determinant thereof, or a polynucleotide coding for a Pemphigus autoantigen or bystander antigen or antigenic determinant thereof.

According to this aspect of the invention there is further provided a pharmaceutical kit comprising a modulator of the Notch signalling pathway and a Pemphigus autoantigen or bystander antigen or antigenic determinant thereof, or a polynucleotide coding for a Pemphigus autoantigen or bystander antigen or antigenic determinant thereof.

In an alternative embodiment of the present invention the autoantigen or bystander antigen may be a Wegener's autoantigen or bystander antigen or antigenic determinant thereof for treatment of Wegener's disease.

According to this aspect of the invention there is further provided a product comprising a modulator of the Notch signalling pathway and a Wegener's autoantigen or bystander antigen or antigenic determinant thereof, or a polynucleotide coding for a Wegener's autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Wegener's autoantigen or bystander antigen or antigenic determinant thereof, or a polynucleotide coding for a Wegener's autoantigen or bystander antigen or antigenic determinant thereof.

According to this aspect of the invention there is further provided a combination of a modulator of the Notch signalling pathway and a Wegener's autoantigen or bystander antigen or antigenic determinant thereof, or a polynucleotide coding for a Wegener's autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Wegener's autoantigen or bystander antigen or antigenic determinant thereof, or a polynucleotide coding for a Wegener's autoantigen or bystander antigen or antigenic determinant thereof.

According to this aspect of the invention there is further provided the use of a combination of a modulator of the Notch signalling pathway and a Wegener's autoantigen or bystander antigen or antigenic determinant thereof, or a polynucleotide coding for a Wegener's autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Wegener's autoantigen or bystander antigen or antigenic determinant thereof, or a polynucleotide coding for a Wegener's autoantigen or bystander antigen or antigenic determinant thereof.

According to this aspect of the invention there is further provided a pharmaceutical kit comprising a modulator of the Notch signalling pathway and a Wegener's autoantigen or bystander antigen or antigenic determinant thereof, or a polynucleotide coding for a Wegener's autoantigen or bystander antigen or antigenic determinant thereof.

In an alternative embodiment of the present invention the autoantigen or bystander antigen may be an autoimmune anemia autoantigen or bystander antigen or antigenic determinant thereof, for treatment of autoimmune anemia.

According to this aspect of the invention there is further provided a product comprising a modulator of the Notch signalling pathway and a autoimmune anemia autoantigen or bystander antigen or antigenic determinant thereof, or a polynucleotide coding for a autoimmune anemia autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and a effective amount of an autoimmune anemia autoantigen or bystander antigen or antigenic determinant thereof, or a polynucleotide coding for an autoimmune anemia autoantigen or bystander antigen or antigenic determinant thereof.

According to this aspect of the invention there is further provided a combination of a modulator of the Notch signalling pathway and an autoimmune anemia autoantigen or bystander antigen or antigenic determinant thereof, or a polynucleotide coding for an autoimmune anemia autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune anemia autoantigen or bystander antigen or antigenic determinant thereof, or a polynucleotide coding for an autoimmune anemia autoantigen or bystander antigen or antigenic determinant thereof.

According to this aspect of the invention there is further provided the use of a combination of a modulator of the Notch signalling pathway and an autoimmune anemia autoantigen or bystander antigen or antigenic determinant thereof, or a polynucleotide coding for an autoimmune anemia autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune anemia autoantigen or bystander antigen or antigenic determinant thereof, or a polynucleotide coding for an autoimmune anemia autoantigen or bystander antigen or antigenic determinant thereof.

According to this aspect of the invention there is further provided a pharmaceutical kit comprising a modulator of the Notch signalling pathway and an autoimmune anemia autoantigen or bystander antigen or antigenic determinant thereof, or a polynucleotide coding for an autoimmune anemia autoantigen or bystander antigen or antigenic determinant thereof.

In an alternative embodiment of the present invention the autoantigen or bystander antigen may be an autoimmune thrombocytopenia autoantigen or bystander antigen or antigenic determinant thereof, for treatment of autoimmune thrombocytopenia.

According to this aspect of the invention there is further provided a product comprising a modulator of the Notch signalling pathway and an autoimmune thrombocytopenia autoantigen or bystander antigen or antigenic determinant thereof, or a polynucleotide coding for an autoimmune thrombocytopenia autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune thrombocytopenia autoantigen or bystander antigen or antigenic determinant thereof, or a polynucleotide coding for an autoimmune thrombocytopenia autoantigen or bystander antigen or antigenic determinant thereof.

According to this aspect of the invention there is further provided a combination of a modulator of the Notch signalling pathway and an autoimmune thrombocytopenia autoantigen or bystander antigen or antigenic determinant thereof, or a polynucleotide coding for an autoimmune thrombocytopenia autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune thrombocytopenia autoantigen or bystander antigen or antigenic determinant thereof, or a polynucleotide coding for an autoimmune thrombocytopenia autoantigen or bystander antigen or antigenic determinant thereof.

According to this aspect of the invention there is further provided the use of a combination of a modulator of the Notch signalling pathway and an autoimmune thrombocytopenia autoantigen or bystander antigen or antigenic determinant thereof, or a polynucleotide coding for an autoimmune thrombocytopenia autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune thrombocytopenia autoantigen or bystander antigen or antigenic determinant thereof, or a polynucleotide coding for an autoimmune thrombocytopenia autoantigen or bystander antigen or antigenic determinant thereof.

According to this aspect of the invention there is further provided a pharmaceutical kit comprising a modulator of the Notch signalling pathway and an autoimmune thrombocytopenia autoantigen or bystander antigen or antigenic determinant thereof, or a polynucleotide coding for an autoimmune thrombocytopenia autoantigen or bystander antigen or antigenic determinant thereof.

In an alternative embodiment of the present invention the autoantigen or bystander antigen may be an autoimmune gastritis autoantigen or bystander antigen or antigenic determinant thereof, for treatment of autoimmune gastritis.

According to this aspect of the invention there is further provided a product comprising a modulator of the Notch signalling pathway and an autoimmune gastritis autoantigen or bystander antigen or antigenic determinant thereof, or a polynucleotide coding for an autoimmune gastritis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune gastritis autoantigen or bystander antigen or antigenic determinant thereof, or a polynucleotide coding for an autoimmune gastritis autoantigen or bystander antigen or antigenic determinant thereof.

According to this aspect of the invention there is further provided a combination of a modulator of the Notch signalling pathway and an autoimmune gastritis autoantigen or bystander antigen or antigenic determinant thereof, or a polynucleotide coding for an autoimmune gastritis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune gastritis autoantigen or bystander antigen or antigenic determinant thereof, or a polynucleotide coding for an autoimmune gastritis autoantigen or bystander antigen or antigenic determinant thereof.

According to this aspect of the invention there is further provided the use of a combination of a modulator of the Notch signalling pathway and an autoimmune gastritis autoantigen or bystander antigen or antigenic determinant thereof, or a polynucleotide coding for an autoimmune gastritis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune gastritis autoantigen or bystander antigen or antigenic determinant thereof, or a polynucleotide coding for an autoimmune gastritis autoantigen or bystander antigen or antigenic determinant thereof.

According to this aspect of the invention there is further provided a pharmaceutical kit comprising a modulator of the Notch signalling pathway and an autoimmune gastritis autoantigen or bystander antigen or antigenic determinant thereof, or a polynucleotide coding for an autoimmune gastritis autoantigen or bystander antigen or antigenic determinant thereof.

In an alternative embodiment of the present invention the autoantigen or bystander antigen may be an autoimmune hepatitis autoantigen or bystander antigen or antigenic determinant thereof, for treatment of autoimmune hepatitis.

According to this aspect of the invention there is further provided a product comprising a modulator of the Notch signalling pathway and an autoimmune hepatitis autoantigen or bystander antigen or antigenic determinant thereof, or a polynucleotide coding for an autoimmune hepatitis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune hepatitis autoantigen or bystander antigen or antigenic determinant thereof, or a polynucleotide coding for an autoimmune hepatitis autoantigen or bystander antigen or antigenic determinant thereof.

According to this aspect of the invention there is further provided a combination of a modulator of the Notch signalling pathway and an autoimmune hepatitis autoantigen or bystander antigen or antigenic determinant thereof, or a polynucleotide coding for an autoimmune hepatitis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune hepatitis autoantigen or bystander antigen or antigenic determinant thereof, or a polynucleotide coding for an autoimmune hepatitis autoantigen or bystander antigen or antigenic determinant thereof.

According to this aspect of the invention there is further provided the use of a combination of a modulator of the Notch signalling pathway and an autoimmune hepatitis autoantigen or bystander antigen or antigenic determinant thereof, or a polynucleotide coding for an autoimmune hepatitis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune hepatitis autoantigen or bystander antigen or antigenic determinant thereof, or a polynucleotide coding for an autoimmune hepatitis autoantigen or bystander antigen or antigenic determinant thereof.

According to this aspect of the invention there is further provided a pharmaceutical kit comprising a modulator of the Notch signalling pathway and an autoimmune hepatitis autoantigen or bystander antigen or antigenic determinant thereof, or a polynucleotide coding for an autoimmune hepatitis autoantigen or bystander antigen or antigenic determinant thereof.

In an alternative embodiment of the present invention the autoantigen or bystander antigen may be an autoimmune vasculitis autoantigen or bystander antigen or antigenic determinant thereof, for treatment of autoimmune vasculitis.

According to this aspect of the invention there is further provided a product comprising a modulator of the Notch signalling pathway and an autoimmune vasculitis autoantigen or bystander antigen or antigenic determinant thereof, or a polynucleotide coding for an autoimmune vasculitis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune vasculitis autoantigen or bystander antigen or antigenic determinant thereof, or a polynucleotide coding for an autoimmune vasculitis autoantigen or bystander antigen or antigenic determinant thereof.

According to this aspect of the invention there is further provided a combination of a modulator of the Notch signalling pathway and an autoimmune vasculitis autoantigen or bystander antigen or antigenic determinant thereof, or a polynucleotide coding for an autoimmune vasculitis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune vasculitis autoantigen or bystander antigen or antigenic determinant thereof, or a polynucleotide coding for an autoimmune vasculitis autoantigen or bystander antigen or antigenic determinant thereof.

According to this aspect of the invention there is further provided the use of a combination of a modulator of the Notch signalling pathway and an autoimmune vasculitis autoantigen or bystander antigen or antigenic determinant thereof, or a polynucleotide coding for an autoimmune vasculitis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune vasculitis autoantigen or bystander antigen or antigenic determinant thereof, or a polynucleotide coding for an autoimmune vasculitis autoantigen or bystander antigen or antigenic determinant thereof.

According to this aspect of the invention there is further provided a pharmaceutical kit comprising a modulator of the Notch signalling pathway and an autoimmune vasculitis autoantigen or bystander antigen or antigenic determinant thereof, or a polynucleotide coding for an autoimmune vasculitis autoantigen or bystander antigen or antigenic determinant thereof.

In an alternative embodiment of the present invention the autoantigen or bystander antigen may be an ocular autoantigen or bystander antigen or antigenic determinant thereof, for treatment of an autoimmune disease of the eye.

According to this aspect of the invention there is further provided a product comprising a modulator of the Notch signalling pathway and an ocular autoantigen or bystander antigen or antigenic determinant thereof, or a polynucleotide coding for an ocular autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an ocular autoantigen or bystander antigen or antigenic determinant thereof, or a polynucleotide coding for an ocular autoantigen or bystander antigen or antigenic determinant thereof.

According to this aspect of the invention there is further provided a combination of a modulator of the Notch signalling pathway and an ocular autoantigen or bystander antigen or antigenic determinant thereof, or a polynucleotide coding for an ocular autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an ocular autoantigen or bystander antigen or antigenic determinant thereof, or a polynucleotide coding for an ocular autoantigen or bystander antigen or antigenic determinant thereof.

According to this aspect of the invention there is further provided the use of a combination of a modulator of the Notch signalling pathway and an ocular autoantigen or bystander antigen or antigenic determinant thereof, or a polynucleotide coding for an ocular autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an ocular autoantigen or bystander antigen or antigenic determinant thereof, or a polynucleotide coding for an ocular autoantigen or bystander antigen or antigenic determinant thereof.

According to this aspect of the invention there is further provided a pharmaceutical kit comprising a modulator of the Notch signalling pathway and an ocular autoantigen or bystander antigen or antigenic determinant thereof, or a polynucleotide coding for an ocular autoantigen or bystander antigen or antigenic determinant thereof.

In an alternative embodiment of the present invention the autoantigen or bystander antigen may be an adrenal autoantigen or bystander antigen or antigenic determinant thereof, for treatment of an adrenal autoimmune disease.

According to this aspect of the invention there is further provided a product comprising a modulator of the Notch signalling pathway and an adrenal autoantigen or bystander antigen or antigenic determinant thereof, or a polynucleotide coding for an adrenal autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an adrenal autoantigen or bystander antigen or antigenic determinant thereof, or a polynucleotide coding for an adrenal autoantigen or bystander antigen or antigenic determinant thereof.

According to this aspect of the invention there is further provided a combination of a modulator of the Notch signalling pathway and an adrenal autoantigen or bystander antigen or antigenic determinant thereof, or a polynucleotide coding for an adrenal autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an adrenal autoantigen or bystander antigen or antigenic determinant thereof, or a polynucleotide coding for an adrenal autoantigen or bystander antigen or antigenic determinant thereof.

According to this aspect of the invention there is further provided the use of a combination of a modulator of the Notch signalling pathway and an adrenal autoantigen or bystander antigen or antigenic determinant thereof, or a polynucleotide coding for an adrenal autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an adrenal autoantigen or bystander antigen or antigenic determinant thereof, or a polynucleotide coding for an adrenal autoantigen or bystander antigen or antigenic determinant thereof.

According to this aspect of the invention there is further provided a pharmaceutical kit comprising a modulator of the Notch signalling pathway and an adrenal autoantigen or bystander antigen or antigenic determinant thereof or a polynucleotide coding for an adrenal autoantigen or bystander antigen or antigenic determinant thereof.

In an alternative embodiment of the present invention the autoantigen or bystander antigen may be a cardiac autoantigen or bystander antigen or antigenic determinant thereof, for treatment of cardiac autoimmune disease.

According to this aspect of the invention there is further provided a product comprising a modulator of the Notch signalling pathway and a cardiac autoantigen or bystander antigen or antigenic determinant thereof, or a polynucleotide coding for a cardiac autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a cardiac autoantigen or bystander antigen or antigenic determinant thereof, or a polynucleotide coding for a cardiac autoantigen or bystander antigen or antigenic determinant thereof.

According to this aspect of the invention there is further provided a combination of a modulator of the Notch signalling pathway and a cardiac autoantigen or bystander antigen or antigenic determinant thereof, or a polynucleotide coding for a cardiac autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a cardiac autoantigen or bystander antigen or antigenic determinant thereof, or a polynucleotide coding for a cardiac autoantigen or bystander antigen or antigenic determinant thereof.

According to this aspect of the invention there is further provided the use of a combination of a modulator of the Notch signalling pathway and a cardiac autoantigen or bystander antigen or antigenic determinant thereof, or a polynucleotide coding for a cardiac autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a cardiac autoantigen or bystander antigen or antigenic determinant thereof, or a polynucleotide coding for a cardiac autoantigen or bystander antigen or antigenic determinant thereof.

According to this aspect of the invention there is further provided a pharmaceutical kit comprising a modulator of the Notch signalling pathway and a cardiac autoantigen or bystander antigen or antigenic determinant thereof, or a polynucleotide coding for a cardiac autoantigen or bystander antigen or antigenic determinant thereof.

In an alternative embodiment of the present invention the autoantigen or bystander antigen may be a scleroderma or myositis autoantigen or bystander antigen or antigenic determinant thereof, for treatment of scleroderma or myositis.

According to this aspect of the invention there is further provided a product comprising a modulator of the Notch signalling pathway and a scleroderma or myositis autoantigen or bystander antigen or antigenic determinant thereof, or a polynucleotide coding for a scleroderma or myositis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a scleroderma or myositis autoantigen or bystander antigen or antigenic determinant thereof, or a polynucleotide coding for a scleroderma or myositis autoantigen or bystander antigen or antigenic determinant thereof.

According to this aspect of the invention there is further provided a combination of a modulator of the Notch signalling pathway and a scleroderma or myositis autoantigen or bystander antigen or antigenic determinant thereof, or a polynucleotide coding for a scleroderma or myositis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a scleroderma or myositis autoantigen or bystander antigen or antigenic determinant thereof, or a polynucleotide coding for a scleroderma or myositis autoantigen or bystander antigen or antigenic determinant thereof.

According to this aspect of the invention there is further provided the use of a combination of a modulator of the Notch signalling pathway and a scleroderma or myositis autoantigen or bystander antigen or antigenic determinant thereof, or a polynucleotide coding for a scleroderma or myositis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a scleroderma or myositis autoantigen or bystander antigen or antigenic determinant thereof, or a polynucleotide coding for a scleroderma or myositis autoantigen or bystander antigen or antigenic determinant thereof.

According to this aspect of the invention there is further provided a pharmaceutical kit comprising a modulator of the Notch signalling pathway and a scleroderma or myositis autoantigen or bystander antigen or antigenic determinant thereof, or a polynucleotide coding for a scleroderma or myositis autoantigen or bystander antigen or antigenic determinant thereof.

In an alternative embodiment of the present invention the autoantigen or bystander antigen may be a nervous system autoantigen or bystander antigen or antigenic determinant thereof, for use to treat an autoimmune disease of the nervous system.

According to this aspect of the invention there is further provided a product comprising a modulator of the Notch signalling pathway and a nervous system (especially MS) autoantigen or bystander antigen or antigenic determinant thereof, or a polynucleotide coding for a nervous system (especially MS) autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a nervous system (especially MS) autoantigen or bystander antigen or antigenic determinant thereof, or a polynucleotide coding for a nervous system (especially MS) autoantigen or bystander antigen or antigenic determinant thereof.

According to this aspect of the invention there is further provided a combination of a modulator of the Notch signalling pathway and a nervous system (especially MS) autoantigen or bystander antigen or antigenic determinant thereof, or a polynucleotide coding for a nervous system (especially MS) autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a nervous system (especially MS) autoantigen or bystander antigen or antigenic determinant thereof, or a polynucleotide coding for a nervous system (especially MS) autoantigen or bystander antigen or antigenic determinant thereof.

According to this aspect of the invention there is further provided the use of a combination of a modulator of the Notch signalling pathway and a nervous system (especially MS) autoantigen or bystander antigen or antigenic determinant thereof, or a polynucleotide coding for a nervous system (especially MS) autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a nervous system (especially MS) autoantigen or bystander antigen or antigenic determinant thereof, or a polynucleotide coding for a nervous system (especially MS) autoantigen or bystander antigen or antigenic determinant thereof.

According to this aspect of the invention there is further provided a pharmaceutical kit comprising a modulator of the Notch signalling pathway and a nervous system (especially MS) autoantigen or bystander antigen or antigenic determinant thereof, or a polynucleotide coding for a nervous system (especially MS) autoantigen or bystander antigen or antigenic determinant thereof.

In an alternative embodiment of the present invention the autoantigen or bystander antigen may be an autoimmune arthritis autoantigen or bystander antigen or antigenic determinant thereof, for use to treat autoimmune arthritis.

According to this aspect of the invention there is further provided a product comprising a modulator of the Notch signalling pathway and an autoimmune arthritis autoantigen or bystander antigen or antigenic determinant thereof, or a polynucleotide coding for an autoimmune arthritis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune arthritis autoantigen or bystander antigen or antigenic determinant thereof, or a polynucleotide coding for an autoimmune arthritis autoantigen or bystander antigen or antigenic determinant thereof.

According to this aspect of the invention there is further provided a combination of a modulator of the Notch signalling pathway and an autoimmune arthritis autoantigen or bystander antigen or antigenic determinant thereof, or a polynucleotide coding for an autoimmune arthritis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune arthritis autoantigen or bystander antigen or antigenic determinant thereof, or a polynucleotide coding for an autoimmune arthritis autoantigen or bystander antigen or antigenic determinant thereof.

According to this aspect of the invention there is further provided the use of a combination of a modulator of the Notch signalling pathway and an autoimmune arthritis autoantigen or bystander antigen or antigenic determinant thereof, or a polynucleotide coding for an autoimmune arthritis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune arthritis autoantigen or bystander antigen or antigenic determinant thereof, or a polynucleotide coding for an autoimmune arthritis autoantigen or bystander antigen or antigenic determinant thereof.

According to this aspect of the invention there is further provided a pharmaceutical kit comprising a modulator of the Notch signalling pathway and an autoimmune arthritis autoantigen or bystander antigen or antigenic determinant thereof, or a polynucleotide coding for an autoimmune arthritis autoantigen or bystander antigen or antigenic determinant thereof.

In an alternative embodiment of the present invention the autoantigen or bystander antigen may be an autoimmune diabetes autoantigen or bystander antigen or antigenic determinant thereof, for use to treat autoimmune diabetes.

According to this aspect of the invention there is further provided a product comprising a modulator of the Notch signalling pathway and an autoimmune diabetes autoantigen or bystander antigen or antigenic determinant thereof, or a polynucleotide coding for an autoimmune diabetes autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an autoimmune diabetes autoantigen or bystander antigen or antigenic determinant thereof, or a polynucleotide coding for an autoimmune diabetes autoantigen or bystander antigen or antigenic determinant thereof.

According to this aspect of the invention there is further provided a combination of a modulator of the Notch signalling pathway and an autoimmune diabetes autoantigen or bystander antigen or antigenic determinant thereof, or a polynucleotide coding for an autoimmune diabetes autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an autoimmune diabetes autoantigen or bystander antigen or antigenic determinant thereof, or a polynucleotide coding for an autoimmune diabetes autoantigen or bystander antigen or antigenic determinant thereof.

According to this aspect of the invention there is further provided the use of a combination of a modulator of the Notch signalling pathway and an autoimmune diabetes autoantigen or bystander antigen or antigenic determinant thereof, or a polynucleotide coding for an autoimmune diabetes autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an autoimmune diabetes autoantigen or bystander antigen or antigenic determinant thereof or a polynucleotide coding for an autoimmune diabetes autoantigen or bystander antigen or antigenic determinant thereof.

According to this aspect of the invention there is further provided a pharmaceutical kit comprising a modulator of the Notch signalling pathway and an autoimmune diabetes autoantigen or bystander antigen or antigenic determinant thereof, or a polynucleotide coding for an autoimmune diabetes autoantigen or bystander antigen or antigenic determinant thereof.

In an alternative embodiment of the present invention the autoantigen or bystander antigen may be a Myasthenia Gravis autoantigen or bystander antigen or antigenic determinant thereof, for use to treat Myasthenia Gravis.

According to this aspect of the invention there is further provided a product comprising a modulator of the Notch signalling pathway and a Myasthenia Gravis autoantigen or bystander antigen or antigenic determinant thereof, or a polynucleotide coding for a Myasthenia Gravis autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Myasthenia Gravis autoantigen or bystander antigen or antigenic determinant thereof, or a polynucleotide coding for a Myasthenia Gravis autoantigen or bystander antigen or antigenic determinant thereof.

According to this aspect of the invention there is further provided a combination of a modulator of the Notch signalling pathway and a Myasthenia Gravis autoantigen or bystander antigen or antigenic determinant thereof or a polynucleotide coding for a Myasthenia Gravis autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Myasthenia Gravis autoantigen or bystander antigen or antigenic determinant thereof, or a polynucleotide coding for a Myasthenia Gravis autoantigen or bystander antigen or antigenic determinant thereof.

According to this aspect of the invention there is further provided the use of a combination of a modulator of the Notch signalling pathway and a Myasthenia Gravis autoantigen or bystander antigen or antigenic determinant thereof, or a polynucleotide coding for a Myasthenia Gravis autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Myasthenia Gravis autoantigen or bystander antigen or antigenic determinant thereof, or a polynucleotide coding for a Myasthenia Gravis autoantigen or bystander antigen or antigenic determinant thereof.

According to this aspect of the invention there is further provided a pharmaceutical kit comprising a modulator of the Notch signalling pathway and a Myasthenia Gravis autoantigen or bystander antigen or antigenic determinant thereof, or a polynucleotide coding for a Myasthenia Gravis autoantigen or bystander antigen or antigenic determinant thereof.

In an alternative embodiment of the present invention the autoantigen or bystander antigen may be a Systemic Lupus Erythematosus (SLE) autoantigen or bystander antigen or antigenic determinant thereof, for use to treat SLE.

According to this aspect of the invention there is further provided a product comprising a modulator of the Notch signalling pathway and a Systemic Lupus Erythematosus autoantigen or bystander antigen or antigenic determinant thereof, or a polynucleotide coding for a Systemic Lupus Erythematosus autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Systemic Lupus Erythematosus autoantigen or bystander antigen or antigenic determinant thereof, or a polynucleotide coding for a Systemic Lupus Erythematosus autoantigen or bystander antigen or antigenic determinant thereof.

According to this aspect of the invention there is further provided a combination of a modulator of the Notch signalling pathway and a Systemic Lupus Erythematosus autoantigen or bystander antigen or antigenic determinant thereof, or a polynucleotide coding for a Systemic Lupus Erythematosus autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Systemic Lupus Erythematosus autoantigen or bystander antigen or antigenic determinant thereof, or a polynucleotide coding for a Systemic Lupus Erythematosus autoantigen or bystander antigen or antigenic determinant thereof.

According to this aspect of the invention there is further provided the use of a combination of a modulator of the Notch signalling pathway and a Systemic Lupus Erythematosus autoantigen or bystander antigen or antigenic determinant thereof, or a polynucleotide coding for a Systemic Lupus Erythematosus autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Systemic Lupus Erythematosus autoantigen or bystander antigen or antigenic determinant thereof, or a polynucleotide coding for a Systemic Lupus Erythematosus autoantigen or bystander antigen or antigenic determinant thereof.

According to this aspect of the invention there is further provided a pharmaceutical kit comprising a modulator of the Notch signalling pathway and a Systemic Lupus Erythematosus autoantigen or bystander antigen or antigenic determinant thereof, or a polynucleotide coding for a Systemic Lupus Erythematosus autoantigen or bystander antigen or antigenic determinant thereof.

In an alternative embodiment of the present invention the autoantigen or bystander antigen may be a bowel autoantigen or bystander antigen or antigenic determinant thereof, for use to treat an autoimmune disease of the bowel.

According to this aspect of the invention there is further provided a product comprising a modulator of the Notch signalling pathway and a bowel autoantigen or bystander antigen or antigenic determinant thereof, or a polynucleotide coding for a bowel autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a bowel autoantigen or bystander antigen or antigenic determinant thereof, or a polynucleotide coding for a bowel autoantigen or bystander antigen or antigenic determinant thereof.

According to this aspect of the invention there is further provided a combination of a modulator of the Notch signalling pathway and a bowel autoantigen or bystander antigen or antigenic determinant thereof, or a polynucleotide coding for a bowel autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a bowel autoantigen or bystander antigen or antigenic determinant thereof, or a polynucleotide coding for a bowel autoantigen or bystander antigen or antigenic determinant thereof.

According to this aspect of the invention there is further provided the use of a combination of a modulator of the Notch signalling pathway and a bowel autoantigen or bystander antigen or antigenic determinant thereof, or a polynucleotide coding for a bowel autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a bowel autoantigen or bystander antigen or antigenic determinant thereof, or a polynucleotide coding for a bowel autoantigen or bystander antigen or antigenic determinant thereof.

According to this aspect of the invention there is further provided a pharmaceutical kit comprising a modulator of the Notch signalling pathway and a bowel autoantigen or bystander antigen or antigenic determinant thereof, or a polynucleotide coding for a bowel autoantigen or bystander antigen or antigenic determinant thereof.

In an alternative embodiment of the present invention the autoantigen or bystander antigen may be a thyroid autoantigen or bystander antigen or antigenic determinant thereof, for use to treat an autoimmune disease of the thyroid.

According to this aspect of the invention there is further provided a product comprising a modulator of the Notch signalling pathway and a thyroid autoantigen or bystander antigen or antigenic determinant thereof, or a polynucleotide coding for a thyroid autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a thyroid autoantigen or bystander antigen or antigenic determinant thereof, or a polynucleotide coding for a thyroid autoantigen or bystander antigen or antigenic determinant thereof.

According to this aspect of the invention there is further provided a combination of a modulator of the Notch signalling pathway and a thyroid autoantigen or bystander antigen or antigenic determinant thereof, or a polynucleotide coding for a thyroid autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a thyroid autoantigen or bystander antigen or antigenic determinant thereof, or a polynucleotide coding for a thyroid autoantigen or bystander antigen or antigenic determinant thereof.

According to this aspect of the invention there is further provided the use of a combination of a modulator of the Notch signalling pathway and a thyroid autoantigen or bystander antigen or antigenic determinant thereof, or a polynucleotide coding for a thyroid autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a thyroid autoantigen or bystander antigen or antigenic determinant thereof, or a polynucleotide coding for a thyroid autoantigen or bystander antigen or antigenic determinant thereof.

According to this aspect of the invention there is further provided a pharmaceutical kit comprising a modulator of the Notch signalling pathway and a thyroid autoantigen or bystander antigen or antigenic determinant thereof, or a polynucleotide coding for a thyroid autoantigen or bystander antigen or antigenic determinant thereof.

In an alternative embodiment of the present invention the autoantigen or bystander antigen may be a Sjogren's autoantigen or bystander antigen or antigenic determinant thereof, for use to treat Sjogren's syndrome.

According to this aspect of the invention there is further provided a product comprising a modulator of the Notch signalling pathway and a Sjogren's autoantigen or bystander antigen or antigenic determinant thereof, or a polynucleotide coding for a Sjogren's autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a Sjogren's autoantigen or bystander antigen or antigenic determinant thereof, or a polynucleotide coding for a Sjogren's autoantigen or bystander antigen or antigenic determinant thereof.

According to this aspect of the invention there is further provided a combination of a modulator of the Notch signalling pathway and a Sjogren's autoantigen or bystander antigen or antigenic determinant thereof, or a polynucleotide coding for a Sjogren's autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a Sjogren's autoantigen or bystander antigen or antigenic determinant thereof, or a polynucleotide coding for a Sjogren's autoantigen or bystander antigen or antigenic determinant thereof.

According to this aspect of the invention there is further provided the use of a combination of a modulator of the Notch signalling pathway and a Sjogren's autoantigen or bystander antigen or antigenic determinant thereof, or a polynucleotide coding for a Sjogren's autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a Sjogren's autoantigen or bystander antigen or antigenic determinant thereof, or a polynucleotide coding for a Sjogren's autoantigen or bystander antigen or antigenic determinant thereof.

According to this aspect of the invention there is further provided a pharmaceutical kit comprising a modulator of the Notch signalling pathway and a Sjogren's autoantigen or bystander antigen or antigenic determinant thereof, or a polynucleotide coding for a Sjogren's autoantigen or bystander antigen or antigenic determinant thereof.

In an alternative embodiment of the present invention the autoantigen or bystander antigen may be an endocrine autoantigen or bystander antigen or antigenic determinant thereof, for use to treat an autoimmune disease of an endocrine gland.

According to this aspect of the invention there is further provided a product comprising a modulator of the Notch signalling pathway and an endocrine autoantigen or bystander antigen or antigenic determinant thereof, or a polynucleotide coding for an endocrine autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of an endocrine autoantigen or bystander antigen or antigenic determinant thereof, or a polynucleotide coding for an endocrine autoantigen or bystander antigen or antigenic determinant thereof.

According to this aspect of the invention there is further provided a combination of a modulator of the Notch signalling pathway and an endocrine autoantigen or bystander antigen or antigenic determinant thereof, or a polynucleotide coding for an endocrine autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with an endocrine autoantigen or bystander antigen or antigenic determinant thereof, or a polynucleotide coding for an endocrine autoantigen or bystander antigen or antigenic determinant thereof.

According to this aspect of the invention there is further provided the use of a combination of a modulator of the Notch signalling pathway and an endocrine autoantigen or bystander antigen or antigenic determinant thereof, or a polynucleotide coding for an endocrine autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with an endocrine autoantigen or bystander antigen or antigenic determinant thereof, or a polynucleotide coding for an endocrine autoantigen or bystander antigen or antigenic determinant thereof.

According to this aspect of the invention there is further provided a pharmaceutical kit comprising a modulator of the Notch signalling pathway and an endocrine autoantigen or bystander antigen or antigenic determinant thereof, or a polynucleotide coding for an endocrine autoantigen or bystander antigen or antigenic determinant thereof.

In an alternative embodiment of the present invention the autoantigen or bystander antigen may be a skin autoantigen or bystander antigen or antigenic determinant thereof, for use to treat an autoimmune disease of the skin.

According to this aspect of the invention there is further provided a product comprising a modulator of the Notch signalling pathway and a skin autoantigen or bystander antigen or antigenic determinant thereof, or a polynucleotide coding for a skin autoantigen or bystander antigen or antigenic determinant thereof, as a combined preparation for simultaneous, contemporaneous, separate or sequential use for modulation of immune response.

According this aspect of the invention there is further provided a method of modulating the immune system in a mammal comprising simultaneously, contemporaneously, separately or sequentially administering to a mammal in need thereof an effective amount of a modulator of the Notch signalling pathway and an effective amount of a skin autoantigen or bystander antigen or antigenic determinant thereof, or a polynucleotide coding for a skin autoantigen or bystander antigen or antigenic determinant thereof.

According to this aspect of the invention there is further provided a combination of a modulator of the Notch signalling pathway and a skin autoantigen or bystander antigen or antigenic determinant thereof, or a polynucleotide coding for a skin autoantigen or bystander antigen or antigenic determinant thereof; for simultaneous, contemporaneous, separate or sequential use in modulating the immune system.

According to this aspect of the invention there is further provided a modulator of the Notch signalling pathway for use in modulating the immune system in simultaneous, contemporaneous, separate or sequential combination with a skin autoantigen or bystander antigen or antigenic determinant thereof, or a polynucleotide coding for a skin autoantigen or bystander antigen or antigenic determinant thereof.

According to this aspect of the invention there is further provided the use of a combination of a modulator of the Notch signalling pathway and a skin autoantigen or bystander antigen or antigenic determinant thereof, or a polynucleotide coding for a skin autoantigen or bystander antigen or antigenic determinant thereof; in the manufacture of a medicament for modulation of immune response.

According to this aspect of the invention there is further provided the use of a modulator of the Notch signalling pathway in the manufacture of a medicament for modulation of immune response in simultaneous, contemporaneous, separate or sequential combination with a skin autoantigen or bystander antigen or antigenic determinant thereof, or a polynucleotide coding for a skin autoantigen or bystander antigen or antigenic determinant thereof.

According to this aspect of the invention there is further provided a pharmaceutical kit comprising a modulator of the Notch signalling pathway and a skin autoantigen or bystander antigen or antigenic determinant thereof, or a polynucleotide coding for a skin autoantigen or bystander antigen or antigenic determinant thereof.

Preferably in any aspect of the present invention, the modulator of the Notch signalling pathway is an agent which activates the Notch signalling pathway, or a polynucleotide which codes for such an agent.

Preferably in any aspect of the present invention, the modulator of the Notch signalling pathway is an agent which activates, preferably directly activates, the Notch receptor (e.g. human Notch1, human Notch2, human Notch3 or human Notch4), or a polynucleotide which codes for such an agent. Preferably the Notch receptor is activated in immune cells, preferably T-cells. In particular, the modulator of Notch signalling is preferably not an agent which acts initially by upregulating expression of a Notch ligand (although this may be an indirect effect of action).

Preferably the modulator of the Notch signalling pathway is not a Notch IC protease, and in particular is preferably not a modulator of presenilin-dependent gamma secretase activity. In a preferred embodiment the modulator of the Notch signalling pathway is not a cytokine.

Suitably the modulator of the Notch signalling pathway may comprise a fusion protein or a polynucleotide which codes for a fusion protein. For example, the modulator may be a fusion protein comprising a segment of a Notch ligand extracellular domain and an immunoglobulin Fc segment or a polynucleotide encoding such a fusion protein.

Suitably in any aspect of the present invention, the modulator of the Notch signalling pathway comprises a protein or polypeptide comprising a Notch ligand DSL or EGF-like domain or a fragment, derivative, homologue, analogue or allelic variant thereof or a polynucleotide sequence coding for such a protein, polypeptide, fragment, derivative, homologue, analogue or allelic variant.

Preferably the modulator of the Notch signalling pathway comprises a Notch ligand DSL domain and at least 1 to 20, suitably at least 2 to 15, suitably at least 2 to 10, for example at least 3 to 8 EGF-like domains. Suitably the DSL and EGF-like domain sequences are or correspond to mammalian sequences. Preferred sequences include human sequences such as human Delta1, Delta3, Delta4, Jagged1 or Jagged2 sequences.

Alternatively or in addition the modulator of the Notch signalling pathway may comprise, for example, Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof. The Notch intracellular domain may, for example, be an active part of the intracellular domain of human Notch1, human Notch2, human Notch3 or human Notch4.

Suitably in any aspect of the present invention, the modulator of the Notch signalling pathway comprises a Notch ligand or a fragment, derivative, homologue, analogue or allelic variant thereof or a polynucleotide encoding a Notch ligand or a fragment, derivative, homologue, analogue or allelic variant thereof.

Suitably in any aspect of the present invention, the modulator of the Notch signalling pathway comprises Delta or a fragment, derivative, homologue, analogue or allelic variant thereof or a polynucleotide encoding Delta or a fragment, derivative, homologue, analogue or allelic variant thereof.

Alternatively or in addition the modulator of the Notch signalling pathway may comprise Serrate/Jagged or a fragment, derivative, homologue, analogue or allelic variant thereof or a polynucleotide encoding Serrate/Jagged or a fragment, derivative, homologue, analogue or allelic variant thereof.

Alternatively or in addition the modulator of the Notch signalling pathway may comprise Notch (e.g. human Notch1, Notch2, Notch3 or Notch4) or a fragment, derivative, homologue, analogue or allelic variant thereof or a polynucleotide encoding Notch or a fragment, derivative, homologue, analogue or allelic variant thereof.

Alternatively or in addition the modulator of the Notch signalling pathway may comprise a dominant negative version of a Notch signalling repressor, or a polynucleotide which codes for a dominant negative version of a Notch signalling repressor.

Suitably a modulator of Notch signalling for use in any aspect of the present invention may comprise a protein or polypeptide comprising:

i) a Notch ligand DSL domain;

ii) 1-5 (and in one embodiment not more than 5) Notch ligand EGF domains;

iii) optionally all or part of a Notch ligand N-terminal domain; and

iv) optionally one or more heterologous amino acid sequences;

or a polynucleotide coding therefor.

Suitably a modulator of Notch signalling for use in any aspect of the present invention may comprise a protein or polypeptide comprising:

i) a Notch ligand DSL domain;

ii) 2-4 (and in one embodiment not more than 4) Notch ligand EGF domains;

iii) optionally all or part of a Notch ligand N-terminal domain; and

iv) optionally one or more heterologous amino acid sequences;

or a polynucleotide coding therefor.

Suitably a modulator of Notch signalling for use in any aspect of the present invention may comprise a protein or polypeptide comprising:

i) a Notch ligand DSL domain;

ii) 2-3 (and in one embodiment not more than 3) Notch ligand EGF domains;

iii) optionally all or part of a Notch ligand N-terminal domain; and

iv) optionally one or more heterologous amino acid sequences;

or a polynucleotide coding therefor.

Suitably such a protein or polypeptide may have at least 50%, preferably at least 70%, preferably at least 90%, for example at least 95% amino acid sequence similarity (or preferably sequence identity) to the following sequence along the entire length of the latter (SEQ ID NO:1):

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCIC
NGATCTNTGQGSYTCSCRPGYTGATCELGIDEC

Preferably a modulator of Notch signalling will be in a multimerised form, and may preferably comprise a construct comprising at least 3, preferably at least 5, preferably at least 10, at least 30, or at least 50 or 100 or more modulators of Notch signalling.

For example, modulators of Notch signalling in the form of Notch ligand proteins/polypeptides coupled to particulate supports such as beads are described in WO 03/011317 (Lorantis) and in Lorantis' co-pending PCT application PCT/GB2003/001525 (filed on 4 Apr.2003), published as WO 03087159, the texts of which are hereby incorporated by reference (e.g. see in particular Examples 17, 18, 19 of PCT/GB2003/001525); and Lorantis Ltd's co-pending PCT application filed on 7 Jan. 2004 claiming priority from GB 0300234.2, the text of which is also hereby incorporated by reference.

Modulators of Notch signalling in the form of Notch ligand proteins/polypeptides coupled to polymer supports are described in Lorantis Ltd's co-pending PCT application PCT/GB2003/003285 (filed on 1 Aug. 2003 claiming priority from GB 0218068.5), the text of which is herein incorporated by reference (e.g. see in particular Example 5 therein disclosing a dextran conjugate)

Alternatively the modulator of the Notch signalling pathway may comprise an antibody, antibody fragment or antibody derivative or a polynucleotide which codes for an antibody, antibody fragment or antibody derivative.

For example, antibodies against Notch and Notch ligands are described in U.S. Pat. No. 5,648,464, U.S. Pat. No. 5,849,869 and U.S. Pat. No. 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated C11 having the ATCC Accession No. HB12656 and a monoclonal antibody secreted by a hybridoma designated F3 having the ATCC Accession No. HB12655.

An anti-human-Jagged1 antibody is available from R & D Systems, Inc, reference MAB12771 (Clone 188323).

According to a further aspect of the invention there is provided a conjugate comprising first and second sequences, wherein the first sequence comprises an autoantigen or bystander antigen or a polynucleotide sequence coding for such an antigen or antigenic determinant and the second sequence comprises a polypeptide or polynucleotide for Notch signalling modulation.

Suitably the conjugate may be in the form of a nucleotide vector, preferably an expression vector, comprising a first polynucleotide sequence coding for an activator of the Notch signalling pathway (such as a Notch ligand or active fragment thereof) and a second polynucleotide sequence coding for an autoantigen or bystander antigen or antigenic determinat thereof. Suitable vectors include vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Alternatively, two or more separate vectors may be used, such that a first vector comprises a polynucleotide sequence coding for a modulator of the Notch signalling pathway and a second vector comprises a polynucleotide sequence coding for an autoantigen or bystander antigen antigenic determinant thereof. Suitably such vectors may be co-coated onto particles for delivery as described infra under the heading “Particles and Particle Delivery”.

According to a further aspect of the invention there is provided a method for producing a lymphocyte or antigen presenting cell (APC) capable of promoting tolerance to an autoantigen or bystander antigen which method comprises incubating a lymphocyte or APC obtained from a human or animal patient with (i) a modulator of the Notch signalling pathway and (ii) an autoantigen or bystander antigen or antigenic determinant thereof, or a polynucleotide coding for an autoantigen or bystander antigen or antigenic determinant thereof.

Suitably the method comprises incubating a lymphocyte or APC obtained from a human or animal patient with an APC in the presence of (i) a modulator of the Notch signalling pathway and (ii) an autoantigen or bystander antigen or antigenic determinant thereof or a polynucleotide coding for an autoantigen or bystander antigen or antigenic determinant thereof.

According to a further aspect of the invention there is provided a method for producing an APC capable of inducing tolerance in a T cell to an autoantigen or bystander antigen which method comprises contacting an APC with (i) a modulator of the Notch signalling pathway and (ii) an autoantigen or bystander antigen or antigenic determinant thereof or a polynucleotide coding for an autoantigen or bystander antigen or antigenic determinant thereof.

According to a further aspect of the invention there is provided a method for producing a T cell capable of promoting tolerance to an autoantigen or bystander antigen which method comprises incubating an antigen presenting cell (APC) simultaneously or sequentially, in any order, with:

(i) an autoantigen or bystander antigen or antigenic determinant thereof or a polynucleotide coding for an autoantigen or bystander antigen or antigenic determinant thereof;

(ii) a modulator of the Notch signalling pathway; and

(iii) a T cell obtained from a human or animal patient.

According to a further aspect of the invention there is provided a method for producing a lymphocyte or APC capable of promoting tolerance to an autoantigen or bystander antigen or antigenic determinant thereof, which method comprises incubating a lymphocyte or APC obtained from a human or animal patient with a lymphocyte or APC produced as described above.

Suitably in such methods the lymphocyte or APC is incubated ex-vivo.

According to a further aspect of the invention there is provided a method of promoting tolerance to an autoantigen or bystander antigen, which method comprises administering to the patient a lymphocyte or APC produced by a method as described above.

The term “APC” as used herein, includes any vehicle capable of presenting the desired Notch-ligand to the T cell population. Examples of suitable APCs include dendritic cells, L cells, hybridomas, lymphomas, macrophages, B cells or synthetic APCs such as lipid membranes.

When the APCs are transfected with a gene capable of expressing a Notch-ligand, the transfection may be brought about by a virus such as a retrovirus or adenovirus, or by any other vehicle or method capable of delivering a gene to the cells. These include any vehicles or methods shown to be effective in gene therapy and include retroviruses, liposomes, electroporation, other viruses such as adenovirus, adeno-associated virus, herpes virus, vaccinia, calcium phosphate precipitated DNA, DEAE dextran assisted transfection, microinjection, nucleofection, polyethylene glycol, protein-DNA complexes.

It will be appreciated that the “bystander effect” described herein is of general application beyond autoimmune disease, and can also be applied for example in treatment or prevention of allergy and graft rejection.

According to a further aspect of the invention there is provided a method for reducing an immune response to a target disease antigen or antigenic determinant thereof by administering a bystander antigen or antigenic determinant thereof (or a polynucleotide coding for such an antigen or antigenic determinant) and simultaneously, separately or sequentially administering an activator of Notch signalling.

According to a further aspect of the invention there is provided a method for reducing an immune response to a target disease autoantigen or antigenic determinant thereof, by administering a bystander antigen or antigenic determinant thereof (or a polynucleotide coding for such an antigen or antigenic determinant) and simultaneously, separately or sequentially administering an activator of Notch signalling.

According to a further aspect of the invention there is provided a product for reducing an immune response to a target disease antigen or antigenic determinant thereof comprising i) a bystander antigen or antigenic determinant thereof (or a polynucleotide coding for such an antigen or antigenic determinant) and ii) an activator of Notch signalling, for simultaneous, separate or sequential administration for reducing an immune response to a target disease antigen.

According to a further aspect of the invention there is provided the use of an activator of Notch signaling in simultaneous, separate or sequential combination with a bystander antigen or antigenic determinant thereof (or a polynucleotide coding for such an antigen or antigenic determinant) for reducing an immune response to a target antigen.

The term “target disease antigen” herein preferably means an antigen, which may or may not be explicitly identified, which is presented as part of an immune disease process, preferably being presented in an affected locus (e.g. organ or tissue) or lymphatic tissues draining this locus, together with one or more bystander antigens, wherein an unwanted or overly severe immune response against such target disease antigen contributes significantly to an immune disease or disorder. The antigen may for example be an autoantigen, allergen or graft antigen.

The term “bystander antigen” herein preferably means an antigen presented as part of an immune disease process, preferably being presented in an affected locus (e.g. organ or tissue) or lymphatic tissues draining this locus, together with a target antigen, whether or not the bystander antigen contributes significantly to an unwanted or overly severe immune response.

In one embodiment the “bystander antigen” is not the or a primary causative antigen of the relevant disease state and may not itself contribute significantly to unwanted or overly severe immune response, but is frequently present at the site of that response (disease locus) as a “bystander”.

Alternatively, the bystander antigen may be an exogenous (foreign) antigen or antigenic determinant (e.g. KLH or any other suitable exogenous antigen) that is delivered to the affected target tisse (e.g. by direct physical introduction, such as by injection or other such means, or targeted with an agent which concentrates it at the requires site, such as an antibody specific for an antigen present at the target site) to trigger suppressive immune cells (preferably T-cells, preferably regulatory T-cells) in the target tissue or lymphatic tissues draining this tissue.

Thus, according to a further aspect of the invention there is provided a method for generating immune suppression at a disease locus by:

i) administering an exogenous antigen or antigenic determinant thereof (or a polynucleotide coding for such an exogenous antigen or antigenic determinant) and simultaneously, separately or sequentially administering an activator of Notch signalling and

ii) administering or targeting said exogenous antigen or antigenic determinant (or a polynucleotide coding for such an antigen or antigenic determinant) to the disease locus to generate bystander immune suppression in said locus.

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic representation of the Notch signalling pathway;

FIG. 2 shows schematic representations of the Notch ligands Jagged and Delta;

FIG. 3 shows an example of a nucleotide vector according to one embodiment of the present invention;

FIG. 4 shows aligned amino acid sequences of DSL domains from various Drosophila and mammalian Notch ligands (SEQ ID NOs:2-17);

FIGS. 5A-5C show amino acid sequences of human Delta-1 (SEQ ID NO:18), Delta-3 (SEQ ID NO:19) and Delta-4 (SEQ ID NO:20);

FIGS. 6A and 6B show amino acid sequences of human Jagged-1 (SEQ ID NO:21) and Jagged-2 (SEQ ID NO:22);

FIG. 7 shows schematic representations of fusion proteins which may be used in the present invention;

FIGS. 8 and 9 show results from Example 5; and

FIG. 10 shows results from Example 7.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

For the avoidance of doubt, Drosophila and vertebrate names for genes and proteins are used interchangeably and all homologues are included within the scope of the invention.

Autoantigens and Bystander Antigens

The term “autoantigen” as used herein includes any substance or a component thereof normally found within a mammal that, in an autoimmune disease, becomes a target of attack by the immune system, preferably the primary (or a primary) target of attack. The term also includes antigenic substances that induce conditions having the characteristics of an autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction, such as heatshock proteins (HSP), which although not necessarily specific to a particular tissue are normally shielded from the immune system.

“Bystander suppression” is suppression at the locus of autoimmune attack of cells that contribute to autoimmune destruction; without wishing to be bound by any theory of mode of action, it is believed that this suppression may be mediated at least in part by the release of one or more immunosuppressive factors (including Th2-enhancing cytokines and Th1-inhibiting cytokines) from suppressor/regulatory T-cells elicited by a bystander antigen and recruited to the site where cells contributing to autoimmune destruction are found. The result may for example be antigen-nonspecific but locally restricted downregulation of the autoimmune responses responsible for tissue destruction.

“Autoimmune disease” includes spontaneous or induced malfunction of the immune system of mammals, including humans, in which the immune system fails to distinguish between foreign immunogenic substances within the mammal and/or autologous substances and, as a result, treats autologous tissues and substances as if they were foreign and mounts an immune response against them.

Autoimmune diseases are characterized by immune responses that are directed against self antigens. These responses are maintained by the persistent activation of self-reactive T lymphocytes. T lymphocytes are specifically activated upon recognition of foreign and/or self antigens as a complex with self Major Histocompatibility Complex (MHC) gene products on the surface of antigen-presenting cells (APC).

A detailed discussion of autoimmune diseases, autoantigens and bystander antigens is included in the textbook “The Autoimmune Diseases” Third Edition, 1998, edited by Rose and Mackay, Academic Press, San Diego, Calif., US (Library of Congress Card Catalog No 98-84368, ISBN 0-12-596923-6), the text of which is hereby incorporated herein by reference.

A non-limiting list of autoimmune diseases and tissue- or organ-specific confirmed or potential bystander antigens and autoantigens effective in the treatment of these diseases is provided below.

Autoimmune Disorders

Autoimmune disorders include organ specific diseases and systemic illnesses.

In more detail, organ-specific autoimmune diseases include, for example, several forms of anemia (aplastic, hemolytic), autoimmune hepatitis, iridocyclitis, scleritis, uveitis, orchitis and idiopathic thrombocytopenic purpura.

Systemic autoimmune diseases include, for example: undifferentiated connective tissue syndrome, antiphospholipid syndrome, different forms of vasculitis (polyarteritis nodosa, allergic granulomatosis and angiitis), Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans, polymyalgia rheumatica, essential (mixed) cryoglobulinemia, psoriasis vulgaris and psoriatic arthritis, diffuse fasciitis with or without eosinophilia, relapsing panniculitis, relapsing polychondritis, lymphomatoid granulomatosis, erythema nodosum, ankylosing spondylitis, Reiter's syndrome and different forms of inflammatory dermatitis.

A more extensive list of disorders includes: unwanted immune reactions and inflammation hepatic fibrosis, liver cirrhosis or other hepatic diseases, thyroiditis, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammation associated with autoimmune diseases or conditions or disorders where, both in the central nervous system (CNS) or in any other organ, immune and/or inflammation suppression would be beneficial, Parkinson's disease, complications and/or side effects from treatment of Parkinson's disease, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems.

Autoantigens

Autoimmune antigens may be derived from tissues, proteins etc. associated with the disease which give rise to the relevant autoimmune response. For example:

| Autoimmune condition | Source of autoantigens |
|---|---|
| Addison's disease | adrenal cell antigens; 21-hydroxylase, 17-hydroxylase |
| Alopecia | hair follicle antigens |
| Autoimmune hepatitis | liver cell antigens |
| Autoimmune parotitis | parotid gland antigens |
| Autoimmune haemolytic anemia | red cell membrane proteins; 95-110 kDa membrane protein |
| Chronic active hepatitis | liver cell antigens |
| Goodpasture's syndrome | renal and lung basement membrane antigens; collagens |
| Guillain-Barre syndrome | nerve cell antigens |
| Hypophysial insufficiency | Hypophyseal antigens |
| Biermer's gastritis | Parietal cell of the stomach; intrinsic Factor |
| Idiopathic leukopenia | granulocyte antigens |
| Idiopathic thrombocytopenia | platelet membrane proteins; Glycoprotein IIa/IIIb |
| Isaac's syndrome | voltage-gated potassium channels |
| Lambert-Eaton | synaptogamin in voltage-gated myasthenic syndrome |
| | (LEMS) calcium channels |
| Myocardial infraction | heart cell antigens |
| Paraneoplastic encephalitis | RNA-binding protein (HuD) |
| Pemphigus vulgaris | “PeV antigen complex”; desmoglein (DG) (see e.g. Eur. J. |
| | Cell Biol. 55: 200 (91)) |
| Primary biliary cirrhosis | mitochondrial antigens; dihydrolipoamide |
| | acetyltransferase; pyruvate dehydrogenase complex 2 |
| | (PDC-E2) |
| Progressive systemic sclerosis | DNA topoisomerase; RNA polymerase |
| Spontaneous infertility | Sperm antigens (e.g. post-acrosomal sperm protein (PASP)) |
| | (see e.g. Biol. Reprod. 43: 559 (90)) |
| Uveitis | Ocular antigen, S-antigen, interphotoreceptor retinoid |
| | binding protein (see e.g. Exp. Eye Res. 56: 463 (93)) |
| Vitiligo | melanocyte antigens |

It will be appreciated that combinations of such autoantigens and autoimmune antigenic determinants and/or polynucleotide sequences coding for them may also be used as appropriate.

An antigen suitable for use in the present invention may be any substance that can be recognised by the immune system, and is generally recognised by an antigen (T-cell) receptor. Preferably the antigen used in the present invention is an immunogen.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component.

The antigen moiety may be, for example, a synthetic MHC-peptide complex i.e. a fragment of the MHC molecule bearing the antigen groove bearing an element of the antigen. Such complexes have been described in Altman et al. (1996) Science 274: 94-96.

Goodpasture's Autoantigens and Bystander Antigens

In one embodiment of the present invention the autoantigen or bystander antigen may be a Goodpasture's autoantigen or bystander antigen for treatment of Goodpasture's disease.

The term “Goodpasture's autoantigen” as used herein includes any substance or a component thereof normally found within a mammal that, in Goodpasture's disease, becomes a target of attack by the immune system, preferably the primary (or a primary) target of attack. The term also includes antigenic substances that induce conditions having the characteristics of Goodpasture's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Goodpasture's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Goodpasture's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Goodpasture's autoantigens and Goodpasture's bystander antigens include, but are not limited to collagens in particular, type IV, alpha 3 collagens.

An amino acid sequence for a human collagen, type IV, alpha 3 (Goodpasture antigen) is reported as follows (GenBank Accession No NM_{—}001723; SEQ ID NO:23):

MSARTAPRPQVLLLPLLLVLLAAAPAASKGCVCKDKGQCFCDGAKGEKGE
KGFPGPPGSPGQKGFTGPEGLPGPQGPKGFPGLPGLTGSKGVRGISGLPG
FSGSPGLPGTPGNTGPYGLVGVPGCSGSKGEQGFPGLPGTPGYPGIPGAA
GLKGQKGAPAKGEDIELDAKGDPGLPGAPGPQGLPGPPGFPGPVGPPGPP
GFFGFPGAMGPRGPKGHMGERVIGHKGERGVKGLTGPPGPPGTVIVTLTG
PDNRTDLKGEKGDKGAMGEPGPPGPSGLPGESYGSEKGAPGDPGLQGKPG
KDGVPGFPGSEGVKGNRGFPGLMGEDGIKGQKGDIGPPGFRGPTEYYDTY
QEKGDEGTPGPPGPRGARGPQGPSGPPGVPGSPGSSRPGLRGAPGWPGLK
GSKGERGRPGKDAMGTPGSPGCAGSPGLPGSPGPPGPPGDIVFRKGPPGD
HGLPGYLGSPGIPGVDGPKGEPGLLCTQCPYIPGPPGLPGLPGLHGVKGI
PGRQGAAGLKGSPGSPGNTGLPGFPGFPGAQGDPGLKGEKGETLQPEGQV
GVPGDPGLRGQPGRKGLDGIPGTLGVKGLPGPKGETALSGEKGDQGPPGD
PGSPGSPGPAGPAGPPGYGPQGEPGLQGTQGVPGAPGPPGEAGPRGELSV
STPVPGPPGPPGPPGHPGPQGPPGIPGSLGKCGDPGLPGPDGEPGIPGIG
FPGPPGPKGDQGFPGTKGSLGCPGKMGEPGLPGKPGLPGAKGEPAVAMPG
GPGTPGFPGERGNSGEEGEIGLPGLPGLPGTPGNEGLDGPRGDPGQPGPP
GEQGPPGRCIEGPRGAQGLPGLNGLKGQQGRRGKTGPKGDPGIPGLDRSG
FPGETGSPGIPGHQGEMGPLGQRGYPGNPGILGPPGEDGVIGMMGFPGAI
GPPGPPGNPGTPGQRGSPGIPGVKGQRGTPGAKGEQGDKGNPGPSEISHV
IGDKGEPGLKGFAGNPGEKGNRGVPGMPGLKGLKGLPGPAGPPGPRGDLG
STGNPGEPGLRGIPGSMGNMGMPGSKGKRGTLGFPGRAGRPGLPGIHGLQ
GDKGEPGYSEGTRPGPPGPTGDPGLPGDMGKKGEMGQPGPPGHLGPAGPE
GAPGSPGSPGLPGKPGPHGDLGFKGIKGLLGPPGIRGPPGLPGFPGSPGP
MGIRGDQGRDGIPGPAGEKGETGLLRAPPGPRGNPGAQGAKGDRGAPGFP
GLPGRKGANGDAGPRGPTGIEGFPGPPGLPGAIIPGQTGNRGPPGSRGSP
GAPGPPGPPGSNVIGIKGDKGSMGHPGPKGPPGTAGDMGPPGRLGAPGTP
GLPGPRGDPGFQGFPGVKGEKGNPGFLGSIGPPGPIGPKGPPGVRGDPGT
LKIISLPGSPGPPGTPGEPGMQGEPGPPGPPGNLGPCGPRGKPGKDGKPG
TPGPAGEKGNKGSKGEPESLFHQL

(See also Turner et al., Molecular cloning of the human Goodpasture antigen demonstrates it to be the alpha 3 chain of type IV collagen, J. Clin. Invest. 89 (2), 592-601 (1992).)

Further sequences are provided, for example, under GenBank Accession Nos. NM_{—}031366.1, NM_{—}031364.1, NM_{—}031363.1, NM_{—}031362.1 and NM_{—}000091.2 (collagen, type IV, alpha 3 (Goodpasture antigen) (COL4A3)) and NM_{—}130778.1 and NM_{—}000494.2 (collagen, type XVII, alpha 1 (COL17A1)).

Renal Autoantigens and Bystander Antigens

In one embodiment the autoantigen or bystander antigen may be a renal autoantigen or renal bystander antigen for treatment of an autoimmune disease of the kidney.

The term “autoimmune disease of the kidney” as used herein includes any disease in which the kidney or renal system or a component thereof comes under autoimmune attack.

The term “renal autoantigen” as used herein includes any substance or a component thereof normally found within a mammal that, in autoimmune disease of the kidney, becomes a target of attack by the immune system, preferably the primary (or a primary) target of attack. The term also includes antigenic substances that induce conditions having the characteristics of an autoimmune disease of the kidney when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “renal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the kidney under autoimmune attack in an autoimmune disease of the kidney. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of renal autoantigens and renal bystander antigens include, but are not limited to glomerular basement membrane (GBM) antigens (Goodpasture's antigens as described further above) and tubular basement membrane (TBM) antigens associated with tubulointerstitial nephritis (TIN). Pemphigus Autoantigens and Bystander Antigens

In an alternative embodiment of the present invention the autoantigen or bystander antigen may be a Pemphigus autoantigen or bystander antigen for treatment of Pemphigus.

The term “Pemphigus autoantigen” as used herein includes any substance or a component thereof normally found within a mammal that, in Pemphigus, becomes a target of attack by the immune system, preferably the primary (or a primary) target of attack. The term also includes antigenic substances that induce conditions having the characteristics of Pemphigus when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Pemphigus bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Pemphigus. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Pemphigus includes, for example, pemphigus vulgaris, pemphigus foliaceus and bullous pemphigoid. Examples of Pemphigus autoantigens and Pemphigus bystander antigens include, but are not limited to desmogleins such as desmoglein 1 and desmoglein 3.

An amino acid sequence for a human desmoglein 1 (DSG1) autoantigen protein is reported as follows (GenBank Accession No AF097935; SEQ ID NO:24):

MDWSFFRVVAVLFIFLVVVEVNSEFRIQVRDYNTKNGTIKWHSIRRQKRE
WIKFAAACREGEDNSKRNPIAKIHSDCAANQQVTYRISGVGIDQPPYGIF
VINQKTGEINITSIVDREVTPFFIIYCRALNSMGQDLERPLELRVRVLDI
NDNPPVFSMATFAGQIEENSNANTLVMILNATDADEPNNLNSKTAFKIIR
QEPSDSPMFIINRNTGEIRTMNNFLDREQYGQYALAVRGSDRDGGADGMS
AECECNIKILDVNDNIPYMEQSSYTIEIQENTLNSNLLEIRVIDLDEEFS
ANWMAVIFFISGNEGNWFEIEMNERTNVGILKVVKPLDYEANQSLQLSIG
VPNKAEFHHSIMSQYKLKASAISVTVLNVIEGPVFRPGSKTYVVTGNMGS
NDKVGDFVATDLDTGRPSTTVRYVMGNNPADLLAVDSRTGKLTLKNKVTK
EQYNMLGGKYQGTILSIDDNLQRTCTGTINIHIQSFGNDDRTNTEPNTKI
TTNTGRQESTSSTNYDTSTTSTDSSQVYSSEPGNGAKDLLSDNVHFGPAG
IGLLIMGFLVLGLVPFLMICCDCGGAPRSAAGFEPVPECSDGAIHSWAVE
GPQPEPRDITTVIPQIPPDNANIIECIDNSGVYTNEYGGREMQDLGGGER
MTGFELTEGVKTSGMPEICQEYSGTLRRNSMRECREGGLNMNFMESYFCQ
KAYAYADEDEGRPSNDCLLIYDIEGVGSPAGSVGCCSFIGEDLDDSFLDT
LGPKFKKLADISLGKESYPDLDPSWPPQSTEPVCLPQETEPVVSGHPPIS
PHFGTTTVISESTYPSGPGVLHPKPILDPLGYGNVTVTESYTTSDTLKPS
VHVHDNRPASNVVVTERVVGPISGADLHGMLEMPDLRDGSNVIVTERVIA
PSSSLPTSLTIHHPRESSNVVVTERVIQPTSGMIGSLSMHPELANAHNVI
VTERVVSGAGVTGISGTTGISGGIGSSGLVGTSMGAGSGALSGAGISGGG
IGLSSLGGTASIGHMRSSSDHHFNQTIGSASPSTARSRITKYSTVQYSK

(See also Nilles et al., Structural analysis and expression of human desmoglein: a cadherin-like component of the desmosome, J. Cell. Sci. 99 (Pt 4), 809-821 (1991).)

An amino acid sequence for a human bullous pemphigoid antigen 1, 230/240 kDa (BPAG1) is reported as follows (GenBank Accession No NM_{—}001723; SEQ ID NO:25):

MHSSSYSYRSSDSVFSNTTSTRTSLDSNENLLLVHCGPTLINSCISFGSE
SFDGHRLEMLQQIANRVQRDSVICEDKLILAGNALQSDSKRLESGVQFQN
EAEIAGYILECENLLRQHVIDVQILIDGKYYQADQLVQRVAKLRDEIMAL
RNECSSVYSKGRILTTEQTKLMISGITQSLNSGFAQTLHPSLTSGLTQSL
TPSLTSSSMTSGLSSGMTSRLTPSVTPAYTPGFPSGLVPNFSSGVEPNSL
QTLKLMQIRKPLLKSSLLDQNLTEEEINNKFVQDLLNWVDEMQVQLDRTE
WGSDLPSVESHLENHKNVHRAIEEFESSLKEAKISEIQMTAPLKLTYAEK
LHRLESQYAKLLNTSRNQERHLDTLHNFVSRATNELIWLNEKEEEEVAYD
WSERNTNIARKKDYHAELMRELDQKEENTKSVQEIAEQLLLENHPARLTI
EAYRAAMQTQWSWILQLCQCVEQHIKENTAYFEFFNDAKEATDYLRNLKD
AIQRKYSCDRSSSIHKLEDLVQESMEEKEELLQYKSTIANLMGKAKTIIQ
LKPRNSDCPLKTSIPIKAICDYRQIEITIYKDDECVLANNSHPAKWKVIS
PTGNEAMVPSVCFTVPPPNKEAVDLANRIEQQYQNVLTLWHESHINMKSV
VSWHYLINEIDRIRASNVASIKTMLPGEHQQVLSNLQSRFEDFLEDSQES
QVFSGSDITQLEKEVNVCKQYYQELLKSAEREEQEESVYNLYISEVRNIR
LRLENCEDRLIRQIRTPLERDDLHESVFRITEQEKLKKELERLKDDLGTI
TNKCEEFFSQAAASSSVPTLRSELNVVLQNMNQVYSMSSTYIDKLKTVNL
VLKNTQAAEALVKLYETKLCEEEAVIADKNNIENLISTLKQWRSEVDEKR
QVFHALEDELQKAKAISDEMFKTYKERDLDFDWHKEKADQLVERWQNVHV
QIDNRLRDLEGIGKSLKYYRDTYHPLDDWIQQVETTQRKIQENQPENSKT
LATQLNQQKMLVSEIEMKQSKMDECQKYAEQYSATVKDYELQTMTYRAMV
DSQQKSPVKRRRMQSSADLIIQEFMDLRTRYTALVTLMTQYIKFAGDSLK
RLEEEEIKRCKETSEHGAYSDLLQRQKATVLENSKLTGKISELERMVAEL
KKQKSRVEEELPKVREAAENELRKQQRNVEDISLQKIRAESEAKQYRREL
ETIVREKEAAERELERVRQLTIEAEAKRAAVEENLLNFRNQLEENTFTRR
TLEDHLKRKDLSLNDLEQQKNKLMEELRRKRDNEEELLKLIKQMEKDLAF
QKQVAEKQLKEKQKIELEARRKITEIQYTCRENALPVCPITQATSCRAVT
GLQQEHDKQKAEELKQQVDELTAANRKAEQDMRELTYELNALQLEKTSSE
EKARLLKDKLDETNNTLRCLKLELERKDQAEKGYSQQLRELGRQLNQTTG
KAEEAMQEASDLKKIKRMYQLELESLNHEKGKLQREVDRITRAHAVAEKN
IQHLNSQIHSFRDEKELERLQICQRKSDHLKEQFEKSHEQLLQNIKAEKE
NNDKIQRLNEELEKSNECAEMLKQKVEELTRQNNETKLMMQRIQAESENI
VLEKQTIQQRCEALKIQADGFKDQLRSTNEHLHKQTKTEQDFQRKIKCLE
EDLAKSQNLVSEFKQKCDQQNIIIQNTKKEVRNLNAELNASKEEKRRGEQ
KVQLQQAQVQELNNRLKKVQDELHLKTIEEQMTHRKMVLFQEESGKFKQS
AEEFRKKMEKLMESKVITENDISGIRLDFVSLQQENSRAQENAKLCETNI
KELERQLQQYREQMQQGQHMEANHYQKCQKLEDELIAQKREVENLKQKMD
QQIKEREHQLVLLQCEIQKKSTAKDCTFKPDFEMTVKECQHSGELSSRNT
GHLHPTPRSPLLRWTQEPQPLEEKWQHRVVEQIPKEVQFQPPGAPLEKEK
SQQCYSEYFSQTSTELQITFDETNPITRLSEIEKIRDQALNNSRPPVRYQ
DNACEMELVKVLTPLEIAKNKQYDMHTEVTTLKQEKNPVPSAEEWMLEGC
RASGGLKKGDFLKKGLEPETFQNFDGDHACSVRDDEFKFQGLRHTVTARQ
LVEAKLLDMRTIEQLRLGLKTVEEVQKTLNKFLTKATSIAGLYLESTKEK
ISFASAAERIIIDKMVALAFLEAQAATGFIIDPISGQTYSVEDAVLKGVV
DPEFRIRLLEAEKAAVGYSYSSKTLSVFQANENRMLDRQKGKHILEAQIA
SGGVIDPVRGIRVPPEIALQQGLLNNAILQFLHEPSSNTRVFPNPNNKQA
LYYSELLRMCVFDVESQCFLFPFGERNISNLNVKKTHRISVVDTKTGSEL
TVYEAFQRNLIEKSIYLELSGQQYQWKEAMFFESYGHSSHMLTDTKTGLH
FNINEAIEQGTIDKALVKKYQEGLITLTELADSLLSRLVPKKDLHSPVAG
YWLTASGERISVLKASRRNLVDRITALRCLEAQVSTGGIIDPLTGKKYRV
AEALHRGLVDEGFAQQLRQCELVITGIGHPIThKMMSVVEAVNANIINKE
MGIRCLEFQYLTGGLIEPQVHSRLSIEEALQVGIIDVLIATKLKDQKSYV
RNIICPQTKRKLTYKEALEKADFDFHTGLKLLEVSEPLMTGISSLYYSS

(See also, for example, Sawamura et al., Bullous pemphigoid antigen (BPAG1): cDNA cloning and mapping of the gene to the short arm of human chromosome 6, Genomics 8 (4), 722-726 (1990).)

Further sequences are provided, for example, under GenBank Accession Nos. NM_{—}015548.1, NM_{—}020388.2 and NM_{—}001723.2 (Bullous pemphigoid antigen 1 (230/240 kD) (BPAG1)), M91669.1 (Bullous pemphigoid autoantigen BP180), NM_{—}001942.1 (desmoglein 1 (DSG1)) and NM_{—}001944.1 (desmoglein 3 (pemphigus vulgaris antigen; DSG3)).

In one embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | |
|---|---|
| LNSKIAFKIVSQEPA | desmoglein 3 (aa 190-204) |
| | (SEQ ID NO:26) |
| TPMFLLSRNTGEVRT | desmoglein 3 (aa 206-220) |
| | (SEQ ID NO:27) |

Wegener's Autoantigens and Bystander Antigens

In an alternative embodiment of the present invention the autoantigen or bystander antigen may be a Wegener's autoantigen or bystander antigen for treatment of Wegener's disease.

The term “Wegener's autoantigen” as used herein includes any substance or a component thereof normally found within a mammal that, in Wegener's disease, becomes a target of attack by the immune system, preferably the primary (or a primary) target of attack. The term also includes antigenic substances that induce conditions having the characteristics of Wegener's disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Wegener's bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Wegener's disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of Wegener's autoantigens and Wegener's bystander antigens include, but are not limited to myeloblastins such as myeloblastin/proteinase 3.

An amino acid sequence for a Wegener's autoantigen/myeloblastin/proteinase 3 autoantigen is reported as follows (GenBank Accession No M75154; SEQ ID NO:28):

MAHRPPSPALASVLLALLLSGAARAAEIVGGHEAQPHSRPYMASLQMRGN
PGSHFCGGTLIHPSFVLTAPHCLRDIPQRLVNVVLGAHNVRTQEPTQQHF
SVAQVFLNNYDAENKLNDILLIQLSSPANLSASVTSVQLPQQDQPVPHGT
QCLAMGWGRVGAHDPPAQVLQELNVTVVTFFCRPHNICTFVPRRKAGICF
GDSGGPLICDGIIQGIDSFVIWGCATRLFPDFFTRVALYVDWIRSTLRRV
EAKGRP

(See also Labbaye et al., Wegener autoantigen and myeloblastin are encoded by a single mRNA, Proc. Natl. Acad. Sci. U.S.A. 88 (20), 9253-9256 (1991).)

Autoimmune Anemia Autoantigens and Bystander Antigens

In an alternative embodiment of the present invention the autoantigen or bystander antigen may be an autoimmune anemia autoantigen or bystander antigen for treatment of autoimmune anemia.

The term “autoimmune anemia” as used herein includes any disease in which red blood cells (RBCs) or a component thereof come under autoimmune attack. The term includes, for example, autoimmune haemolytic anemia, including both “warm autoantibody type” and “cold autoantibody type”.

The term “autoimmune anemia autoantigen” as used herein includes any substance or a component thereof normally found within a mammal that, in autoimmune anemia, becomes a target of attack by the immune system, preferably the primary (or a primary) target of attack. The term also includes antigenic substances that induce conditions having the characteristics of autoimmune anemia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune anemia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the red blood cells (RBCs) under autoimmune attack in autoimmune anemia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune anemia includes, in particular, autoimmune hemolytic anemia. Examples of autoimmune hemolytic anemia autoantigens and bystander antigens include, but are not limited to Rhesus (Rh) antigens such as E, e or C, red cell proteins and glycoproteins such as red cell protein band 4.1 and red cell membrane band 3 glycoprotein. Further examples include Wr^{b}, En^{a}, Ge, A, B and antigens within the Kidd and Kell blood group systems.

Autoimmune Thrombocytopenia Autoantigens and Bystander Antigens

In an alternative embodiment of the present invention the autoantigen or bystander antigen may be an autoimmune thrombocytopenia autoantigen or bystander antigen for treatment of autoimmune thrombocytopenia.

The term “autoimmune thrombocytopenia autoantigen” as used herein includes any substance or a component thereof normally found within a mammal that, in autoimmune thrombocytopenia, becomes a target of attack by the immune system, preferably the primary (or a primary) target of attack. The term also includes antigenic substances that induce conditions having the characteristics of autoimmune thrombocytopenia when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune thrombocytopenia bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the platelets under autoimmune attack in autoimmune thrombocytopenia. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune thrombocytopenia includes, in particular, autoimmune thrombocytopenia purpura. Examples of autoimmune thrombocytopenia purpura autoantigens and bystander antigens include, but are not limited to platelet glycoproteins such as GPIIb/IIIa and/or GPIb/IX.

For example, an amino acid sequence for a human platelet glycoprotein IIb (GPIIb) is reported as follows (GenBank Accession No M34480; SEQ ID NO:29):

MARALCPLQALWLLEWVLLLLGACAAPPAWALNLDPVQLTFYAGPNGSQF
GFSLDFHKDSHGRVAIVVGAPRTLGPSQEETGGVFLCPWRAEGGQCPSLL
FDLRDETRNVGSQTLQTFKARQGLGASVVSWSDVIVACAPWQHWNVLEKT
EEAEKTPVGSCFLAQPESGRRAEYSPCRGNTLSRIYVENDFSWDKRYCEA
GFSSVVTQAGELVLGAPGGYYFLGLLAQAPVADIFSSYRPGILLWHVSSQ
SLSFDSSNPEYFDGYWGYSVAVGEFDGDLNTTEYVVGAPTWSWTLGAVEI
LDSYYQRLHRLRAEQMASYFGHSVAVTDVNGDGRHDLLVGAPLYMDSRAD
RKLAEVGRVYLFLQPRGPHALGAPSLLLTGTQLYGRFGSAIAPLGDLDRD
GYNDIAVAAPYGGPSGRGQVLVFLGQSEGLRSRPSQVLDSPFPTGSAFGF
SLRGAVDIDDNGYPDLIVGAYGANQVAVYRAQPVVKASVQLLVQDSLNPA
VKSCVLPQTKTPVSCFNIQMCVGATGHNIPQKLSLNAELQLDRQKPRQGR
RVLLLGSQQAGTTLDLDLGGKHSPICHTTMAFLRDEADFRDKLSPIVLSL
NVSLPPTEAGMAPAVVLHGDTHVQEQTRIVLDCGEDDVCVPQLQLTASVT
GSPLLVGADNVLELQMDAANEGEGAYEAELAVHLPQGAHYMRALSNVEGF
ERLICNQKKENETRVVLCELGNPMKKNAQIGIAMLVSVGNLEEAGESVSF
QLQIRSKNSQNPNSKIVLLDVPVRAEAQVELRGNSFPASLVVAAEEGERE
QNSLDSWGPKVEHTYELKNNGPGTVNGLHLSIHLPGQSQPSDLLYILDIQ
PQGGLQCFPQPPVNPLKVDWGLPIPSPSPIHPAHHKRDRRQIFLPEPEQP
SRLQDPVLVSCDSAPCTVVQCDLQEMARGQRAMVTVLAFLWLPSLYQRPL
DQFVLQSHAWFNVSSLPYAVPPLSLPRGEAQVWTQLLRALEERAIPIWWV
LVGVLGGLLLLTILVLANWKVGFFKRNRHTLEEDDEEGE

An amino acid sequence for a human platelet glycoprotein IIIa (GPIIIa) is reported as follows (GenBank Accession No M35999; SEQ ID NO:30):

MRARPRPRPLWVTVLALGALAGVGVGGPNICTTRGVSSCQQCLAVSPMCA
WCSDEALPLGSPRCDLKENLLKDNCAPESIEFPVSEARVLEDRPLSDKGS
GDSSQVTQVSPQRIALRLRPDDSKNFSIQVRQVEDYPVDIYYLMDLSYSM
KDDLWSIQNLGTKLATQMRKLTSNLRIGFGAFVDKPVSPYMYISPPEALE
NPCYDMKTTCLPMFGYKHVLTLTDQVTRFNEEVKKQSVSRNRDAPEGGFD
AIMQATVCDEKIGWRNDASHLLVFTTDAKTHIALDGRLAGIVQPNDGQCH
VGSDNHYSASTTMDYPSLGLMTEKLSQKNINLIFAVTENVVNLYQNYSEL
IPGTTVGVLSMDSSNVLQLIVDAYGKIRSKVELEVRDLPEELSLSFNATC
LNNEVIPGLKSCMGLKIGDTVSFSIEAKVRGCPQEKEKSFTIKPVGFKDS
LIVQVTFDCDCACQAQAEPNSHRCNNGNGTFECGVCRCGPGWLGSQCECS
EEDYRPSQQDECSPREGQPVCSQRGECLCGQCVCHSSDFGKITGKYCECD
DFSCVRYKGEMCSGHGQCSCGDCLCDSDWTGYYCNCTTRTDTCMSSNGLL
CSGRGKCECGSCVCIQPGSYGDTCEKCPTCPDACTFKKECVECKKFDRGA
LHDENTCNRYCRDEIESVKELKDTGKDAVNCTYKNEDDCVVRFQYYEDSS
GKSILYVVEEPECPKGPDILVVLLSVMGAILLIGLAALLIWKLLITIHDR
KEFAKFEEERARAKWDTANNPLYKEATSTFTNITYRGT

Autoimmune Gastritis Autoantigens and Bystander Antigens

In an alternative embodiment of the present invention the autoantigen or bystander antigen may be an autoimmune gastritis autoantigen or bystander antigen for treatment of autoimmune gastritis.

The term “autoimmune gastritis” as used herein includes any disease in which gastric tissue or a component thereof comes under autoimmune attack. The term includes, for example, pernicious anemia.

The term “autoimmune gastritis autoantigen” as used herein includes any substance or a component thereof normally found within a mammal that, in autoimmune gastritis, becomes a target of attack by the immune system, preferably the primary (or a primary) target of attack. The term also includes antigenic substances that induce conditions having the characteristics of autoimmune gastritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune gastritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the gastric tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Autoimmune gastritis includes, in particular, pernicious anemia. Examples of autoimmune gastritis autoantigens and bystander antigens include, but are not limited to parietal cell antigens such as gastric H+/K+ ATPase, (e.g. 100 kDa alpha subunit and 60-90 kDa beta subunit; especially the beta subunit) and intrinsic factor.

For example an amino acid sequence for a human H,K-ATPase beta subunit is reported as follows (GenBank Accession No M75110; SEQ ID NO:31):

MAALQEKKTCGQRMEEFQRYCWNPDTGQMLGRTLSRWVWISLYYVAFYVV
MTGLFALCLYVLMQTVDPYTPDYQDQLRSPGVTLRPDVYGEKGLEIVYNV
SDNRTWADLTQTLHAFLAGYSPAAQEDSINCTSEQYFFQESFRAPNHTKF
SCKFTADMLQNCSGLADPNFGFEEGKPCFIIKMNRIVKFLPSNGSAPRVD
CAFLDQPRELGQPLQVKYYPPNGTFSLHYFPYYGKKAQPHYSNPLVAAKL
LNIPRNAEVAIVCKVMAEHVTFNNPHDPYEGKVEFKLKIEK

(See also GenBank Accession No J05451; human gastric (H+/K+)-ATPase gene and GenBank Accession No M63962; human gastric H,K-ATPase catalytic subunit gene.)

Autoimmune Hepatitis Autoantigens and Bystander Antigens

In an alternative embodiment of the present invention the autoantigen or bystander antigen may be an autoimmune hepatitis autoantigen or bystander antigen for treatment of autoimmune hepatitis.

The term “autoimmune hepatitis” as used herein includes any disease in which the liver or a component of the liver comes under autoimmune attack. The term thus includes, for example, primary biliary cirrhosis (PBC) and primary sclerosing cholangitis.

The term “autoimmune hepatitis autoantigen” as used herein includes any substance or a component thereof normally found within a mammal that, in autoimmune hepatitis, becomes a target of attack by the immune system, preferably the primary (or a primary) target of attack. The term also includes antigenic substances that induce conditions having the characteristics of autoimmune hepatitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune hepatitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune gastritis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of autoimmune hepatitis autoantigens and bystander antigens include, but are not limited to cytochromes, especially cytochrome P450s such as cytochrome P450 2D6, cytochrome P450 2C9 and cytochrome P450 1A2, the asialoglycoprotein receptor (ASGP R) and UDP-glucuronosyltransferases (UGTs).

For example, cDNA encoding human cytochrome P450-2d6 (coding for antigen for AIH Type2a LKM1 antibody) is reported as follows (GenBank Accession No E15820; SEQ ID NO:32):

| | |
|---|---|
| 1 | atggggctag aagcactggt gcccctggcc atgatagtgg ccatcttcct gctcctggtg |
| 61 | gacctgatgc accggcgcca acgctgggct gcacgctacc caccaggccc cctgccactg |
| 121 | cccgggctgg gcaacctgct gcatgtggac ttccagaaca caccatactg cttcgaccag |
| 181 | ttgcggcgcc gacttcggga cgtgttcagc ctgcanctgg cctggacgcc ggtggtcgtg |
| 241 | ctcaatgggc tggcggccgt gcgcgaggcg ctggtgaccc acggcgagga caccgccgac |
| 301 | cgcccgcctg tgcccatcac ccagatcctg ggcttcgggc cgcgttccca aggggtgttc |
| 361 | ctggcgcgct atgggcccgc gtggcgcgag cagaggcgct tctccgtctc caccttgcgc |
| 421 | aacttgggcc tgggcaagaa gtcgctggag cagtgggtga ccgaggaggc ngcctgcctt |
| 481 | tgtgccgcct tcgccaacca ctccggacgc ccctttcgcc ccaacggtct cttggacaaa |
| 541 | gccgtgagca acgtgatcgc ctccctcacc tgcgggcgcc gcttcgagta cgacgaccct |
| 601 | cgcttcctca ggctgctgga cctagctcag gagggactga aggaggagtc gggctttctg |
| 661 | cgcgaggtgc tgaatgctgt ccccgtcctc ctgcatatcc cngcgctggc tggcaaggtc |
| 721 | ctacgcttcc aaaaggcttt cctgacccag ctggatgagc tgctaactga gcacaggatg |
| 781 | acctgggacc cagcccagcc cccccgagac ctgactgagg ccttcctggc agagatggag |
| 841 | aaggccaagg ggaaccctgc gagcagcttc aatgatgaga acctgcgcat agtggtggct |
| 901 | gacctgttct ctgccgggat ggtgaccacc tcgaccacgc tggcctgggg cctcctgctc |
| 961 | atgatcctac atccggatgt gcagcgccgt gtccaacagg agatcgacga cgtgataggg |
| 1021 | caggtgcggc gaccagagat gggtgaccag gctcacatgc cctacaccac tgccgtgatt |
| 1081 | catgaggtgc agcgctttgg ggacatcgtc cccctgggtg tgacccatat gacatcccgt |
| 1141 | gacatcgagg tacagggctt cngcatccct aagggaacga cactcatcac caacctgtca |
| 1201 | tcggtnctga aggatgaggc cgtctgggag aagcccttcc gcttccaccc cgaacacttc |
| 1261 | ctggatgccc agggccactt tgtgaagccg gaggccttcc tgcctttctc agcaggccgc |
| 1321 | cgtgcatgcc tcggggagcc cctggcccgc atggagctct tcctcttctt cacctccctg |
| 1381 | ctgcagcact tcagcttctc ggtgcccact ggacagcccc ggcccagcca ccatggtgtc |
| 1441 | tttgctttcc tggtgagccc atccccctat gagctttgtg ctgtgccccg ctagaatggg |
| 1501 | gtacctagtc cccagcctgc tcctagccca gaggctctaa tgtac |

An amino acid sequence for a human cytochrome P450-1A2 (CYP1A2) is reported as follows (GenBank Accession No AF182274; SEQ ID NO:33):

MALSQSVPFSATELLLASAIFCLVFWVLKGLRPRVPKGLKSPPEPWGWPL
LGHVLTLGKNPHLALSRMSQRYGDVLQIRIGSTPVLVLSRLDTIRQALVR
QGDDFKGRPDLYTSTLITDGQSLTFSTDSGPVWAARRRLAQNALNTFSIA
SDPASSSSCYLEEHVSKEAMALISRLQELMAGPGHFDPYNQVVVSVANVI
GAMCFGQHFPESSDEMLSLVKNTHEFVETASSGNPLDFFPILRYLPNPAL
QRFKAFNQRFLWFLQKTVQEHYQDFDKNSVRDITGALFKHSKKGPRASGN
LIPQEKIVNLVNDVFGAGFDTVTTAISWSLMYLVTKPEIQRKIQKELDTV
IGRERRPRLSDRPQLPYLEAFILETFRHSSFLPFTIPHSTTRDTTLNGFY
IPKKCCVFVNQWQVNHDPELWEDPSEFRPERFLTADGTAINKPLSEKMML
FGMGKRRCIGEVLAKWEIFLFLAILLQQLEFSVPPGVKVDLIPIYGLTMK
HARCEHVQARLRFSIN

Examples of primary biliary cirrhosis (PBC) autoantigens and bystander antigens include, but are not limited to mitochondrial antigens such as pyruvate dehydrogenases (e.g. E1-alpha decarboxylase, E1-beta decarboxylase and E2 acetyltransferase), branched-chain 2-oxo-acid dehydrogenases and 2-oxoglutarate dehydrogenases.
Autoimmune Vasculitis Autoantigens and Bystander Antigens

In an alternative embodiment of the present invention the autoantigen or bystander antigen may be an autoimmune vasculitis autoantigen or bystander antigen for treatment of autoimmune vasculitis.

The term “autoimmune vasculitis” as used herein includes any disease in which blood vessels or a component thereof come under autoimmune attack and includes, for example, large vessel vasculitis such as giant cell arteritis and Takayasu's disease, medium-sized vessel vasculitis such as polyarteritis nodosa and Kawasaki disease and small vessel vasculitis such as Wegener's granulomatosis, Churg-Strauss syndrome, microscopic polyangiitis, Henoch Schonlein purpura, essential cryoglobulinaemic vasculitis and cutaneous leukocytoclastic angiitis.

The term “autoimmune vasculitis autoantigen” as used herein includes any substance or a component thereof normally found within a mammal that, in autoimmune vasculitis, becomes a target of attack by the immune system, preferably the primary (or a primary) target of attack. The term also includes antigenic substances that induce conditions having the characteristics of autoimmune vasculitis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune vasculitis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the blood vessel tissue under autoimmune attack in autoimmune vasculitis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of vasculitis autoantigens and bystander antigens include, but are not limited to basement membrane antigens (especially the noncollagenous domain of the alpha 3 chain of type IV collagen) and endothelial cell antigens.

Ocular Autoantigens and Bystander Antigens

In an alternative embodiment of the present invention the autoantigen or bystander antigen may be an ocular autoantigen or bystander antigen for treatment of an autoimmune disease of the eye.

The term “autoimmune disease of the eye” includes any disease in which the eye or a component thereof comes under autoimmune attack. The term thus includes, for example, cicatricial pemphigoid, uveitis, Mooren's ulcer, Reiter's syndrome, Behcet's syndrome, Vogt-Koyanagi-Harada Syndrome, scleritis, lens-induced uveitis, optic neuritis and giant-cell arteritis.

The term “ocular autoantigen” as used herein includes any substance or a component thereof normally found within the eye of a mammal that, in an autoimmune disease of the eye, becomes a target of attack by the immune system, preferably the primary (or a primary) target of attack. The term also includes antigenic substances that induce conditions having the characteristics of autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “ocular bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the eye under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of ocular autoantigens and bystander antigens include, but are not limited to retinal antigens such as ocular antigen, S-antigen, interphotoreceptor retinoid binding protein (see e.g. Exp. Eye Res. 56:463 (93)) in uveitis and alpha crystallin in lens-induced uveitis.

An amino acid sequence for a human retinal S-antigen (48 KDa protein) is reported as follows (GenBank Accession No X12453; SEQ ID NO:34):

MAASGKTSKSEPNHVIFKKISRDKSVTIYLGNRDYIDHVSQVQPVDGVVL
VDPDLVKGKKVYVTLTCAFRYGQEDVDVIGLTFRRDLYFSRVQVYPPVGA
ASTPTKLQESLLKKLGSNTYPFLLTFPDYLPCSVMLQPAPQDSGKSCGVD
FEVKAFATDSTDAEEDKIPKKSSVRYLIRSVQHAPLEMGPQPRAEATWQF
FMSDKPLHLAVSLNREIYGHGEPIPVTVTVTNNTEKTVKKIKACVEQVAN
VVLYSSDYYVKPVAMEEAQEKVPPNSTLTKTLTLLPLLANNRERRGIALD
GKIKHEDTNLASSTIIKEGIDRTVLGILVSYQIKVKLTVSGFLGELTSSE
VATEVPFRLMHPQPEDPAKESIQDANLVFEEFARHNLKDAGEAEEGKRDK
NDADE

An amino acid sequence for a human alpha crystallin is reported as follows (GenBank Accession No U05569; SEQ ID NO:35):

MDVTIQHPWFKRTLGPFYPSRLFDQFFGEGLFEYDLLPFLSSTISPYYRQ
SLFRTVLDSGISEVRSDRDKFVIFLDVKHFSPEDLTVKVQDDFVEIHGKH
NERQDDHGYISREFHRRYRLPSNVDQSALSCSLSADGMLTFCGPKIQTGL
DATHAERAIPVSREEKPTSAPSS

Adrenal Autoantigens and Bystander Antigens

In an alternative embodiment of the present invention the autoantigen or bystander antigen may be an adrenal autoantigen or bystander antigen for treatment of an adrenal autoimmune disease.

The term “adrenal autoimmune disease” as used herein includes any disease in which the adrenal gland or a component thereof comes under autoimmune attack. The term includes, for example, Addison's disease.

The term “adrenal autoantigen” as used herein includes any substance or a component thereof normally found within a mammal that, in adrenal autoimmune disease, becomes a target of attack by the immune system, preferably the primary (or a primary) target of attack. The term also includes antigenic substances that induce conditions having the characteristics of adrenal autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “adrenal bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the adrenal gland under autoimmune attack in adrenal autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of adrenal autoantigens and bystander antigens include, but are not limited to adrenal cell antigens such as the adrenocorticotropic hormone receptor (ACTH receptor) and enzymes such as 21-hydroxylase and 17-hydroxylase.

For example, an amino acid sequence for a human steroid 17-alpha-hydroxylase is reported as follows (GenBank Accession No NM_{—}000102; SEQ ID NO:36):

MWELVALLLLTLAYLFWPKRRCPGAKYPKSLLSLPLVGSLPFLPRHGHMH
NNFFKLQKKYGPIYSVRMGTKTTVIVGHNQLAKEVLTKKGKDFSGRPQMA
TLDIASNNRKGIAFADSGAHWQLHRRLAMATFALFKDGDQKLEKIICQEI
STLCDMLATHNGQSIDISFPVFVAVTNVISLICFNTSYKNGDPELNVIQN
YNEGIIDNLSKDSLVDLVPWLKIFPNKTLEKLKSHVKIRNDLLNKILENY
KEKFRSDSITNMLDTLMQAKMNSDNGNAGPDQDSELLSDNHILTTIGDIF
GAGVETTTSVVKWTLAFLLHNPQVKKKLYEEIDQNVGFSRTPTISDRNRL
LLLEATIREVLRLRPVAPMLIPHKANVDSSIGEFAVDKGTEVIINLWALH
HNEKEWHQPDQFMPERFLNPAGTQLISPSVSYLPFGAGPRSCIGEILARQ
ELFLIMAWLLQRFDLEVPDDGQLPSLEGIPKVVFLIDSFKVKIKVRQAWR
EAQAEGST

(See also Krohn et al: Identification by molecular cloning of an autoantigen associated with Addison's disease as steroid 17 alpha-hydroxylase, Lancet 339 (8796), 770-773 (1992).)

Cardiovascular Autoantigens and Bystander Antigens

In an alternative embodiment of the present invention the autoantigen or bystander antigen may be a cardiac autoantigen or bystander antigen for treatment of cardiac autoimmune disease.

The term “cardiac autoimmune disease” as used herein includes any disease in which the heart or a component thereof comes under autoimmune attack. The term includes, for example, autoimmune myocarditis, dilated cardiomyopathy, autoimmune rheumatic fever and Chagas' disease.

The term “cardiac autoantigen” as used herein includes any substance or a component thereof normally found within a mammal that, in cardiac autoimmune disease, becomes a target of attack by the immune system, preferably the primary (or a primary) target of attack. The term also includes antigenic substances that induce conditions having the characteristics of cardiac autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “cardiac bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the heart tissue under autoimmune attack in cardiac autoimmune disease. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of cardiac autoantigens and bystander antigens include, but are not limited to heart muscle cell antigens such as mysosins, laminins, beta-1 adrenergic receptors, adenine nucleotide translocator (ANT) protein and branched-chain ketodehydrogenase (BCKD).

Scleroderma/Polymyositis Autoantigens and Bystander Antigens

In an alternative embodiment of the present invention the autoantigen or bystander antigen may be a scleroderma or myositis autoantigen or bystander antigen for treatment of scleroderma or myositis.

The term “myositis/scleroderma autoantigen” as used herein includes any substance or a component thereof normally found within a mammal that, in myositis (particularly in dermatomyositis or polymyositis) or scleroderma, becomes a target of attack by the immune system, preferably the primary (or a primary) target of attack. The term also includes antigenic substances that induce conditions having the characteristics of myositis (particularly in dermatomyositis or polymyositis) or scleroderma when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “myositis/scleroderma bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in myositis (particularly in dermatomyositis or polymyositis) or scleroderma. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

As described, for example, in U.S. Pat. No. 5,862,360, scleroderma, or systemic sclerosis, is characterized by deposition of fibrous connective tissue in the skin, and often in many other organ systems. It may be accompanied by vascular lesions, especially in the skin, lungs, and kidneys. The course of this disease is variable, but it is usually slowly progressive. Scleroderma may be limited in scope and compatible with a normal life span. Systemic involvement, however, can be fatal.

Scleroderma may be classified as either diffuse or limited, on the basis of the extent of skin and internal organ involvement. The diffuse form is characterized by thickening and fibrosis of skin over the proximal extremities and trunk. The heart, lungs, kidneys, and gastrointestinal tract below the esophagus are often involved. Limited scleroderma is characterized by cutaneous involvement of the hands and face. Visceral involvement occurs less commonly. The limited form has a better prognosis than the diffuse form, except when pulmonary hypertension is present.

Antinuclear antibodies are found in over 95 percent of patients with scleroderma. Specific antinuclear antibodies have been shown to be directed to topoisomerase I, centromere proteins, RNA polymerases, or nucleolar components. Different antibodies are associated with particular clinical patterns of scleroderma. For example, antibodies to topoisomerase I (Scl-70) and to RNA polymerases (usually RNA polymerase III) are seen in patients with diffuse scleroderma. Antibodies to nuclear ribonucleoprotein (nRNP) are associated with diffuse and limited scleroderma.

Patients with scleroderma typically show autoreactivity against centrosomes (Tuffanelli, et al., Arch. Dermatol., 119:560-566, 1983). Centrosomes are essential structures that are highly conserved, from plants to mammals, and are important for various cellular processes. Centrosomes play a crucial role in cell division and its regulation. Centrosomes organize the mitotic spindle for separating chromosomes during cell division, thus ensuring genetic fidelity. In most cells, the centrosome includes a pair of centrioles that lie at the center of a dense, partially filamentous matrix, the pericentriolar material (PCM). The microtubule cytoskeleton is anchored to the centrosome or some other form of microtubule organizing center (MTOC), which is thought to serve as a site of microtubule nucleation.

As discussed in U.S. Pat. No. 6,160,107 the idiopathic inflammatory myopathies polymyositis, dermatomyositis and the related overlap syndromes disorder, such as polymyositis-scleroderma overlap, are inflammatory myopathies that are characterized by chronic muscle inflammation and proximal muscle weakness. The muscle inflammation causes muscle tenderness, muscle weakness, and ultimately muscle atrophy and fibrosis (see, for example, Plotz, et al. Annals of Internal Med. 111: 143-157(1989)). Also associated with the muscle inflammation are elevated serum levels of aldolase, creatine kinase, transaminases, such as alanine aminotransferase and aspartate aminotransferase, and lactic dehydrogenase. Other systems besides muscle can be affected by these conditions, resulting in arthritis, Raynaud's phenomenon, and interstitial lung disease. Clinically, polymyositis and dermatomyositis are distinguished by the presence of a characteristic rash in patients with dermatomyositis. Differences in the myositis of these conditions can be distinguished in some studies of muscle pathology.

Autoantibodies can be detected in about 90% of patients with polymyositis and dermatomyositis (Reichlin and Arnett, Arthritis and Rheum. 27: 1150-1156 (1984)). Sera from about 60% of these patients form precipitates with bovine thymus extracts on Ouchterlony immunodiffusion (ID), while sera from other patients stain tissue culture substrates, such as HEp-2 cells, by indirect immunofluorescence (IIF) (see, e.g., Targoff and Reichlin Arthritis and Rheum. 28: 796-803 (1985); Nishikai and Reichlin Arthritis and Rheum. 23: 881-888 (1980); Reichlin, et al., J. Clin. Immunol. 4:40-44 (1984)).

Many autoantibodies associated with myositis or myositis-overlap syndromes have been defined, and, in some cases, the antibodies have been identified. These include antibodies that are present in other disorders and also disease-specific antibodies (see, e.g., (Targoff and Reichlin Mt. Sinai J. of Med. 55: 487-493 (1988)). For example, a group of myositis-associated autoantibodies have been identified which are directed at cytoplasmic proteins that are related to tRNA and protein synthesis, particularly aminoacyl-tRNA synthetases. These include anti-Jo-1, which is the most common autoantibody associated with myositis autoimmune disorders (about 20% of such patients (Nishikai, et al. Arthritis Rheum. 23: 881-888 (1980)) and which is directed against histidyl-tRNA synthetase; anti-PL-7, which is directed against threonyl-tRNA synthetase; and anti-PL12, which is directed against alanyl-tRNA synthetase. Anti-U1 RNP, which is frequently found in patients with SLE, may also be found in mixed connective tissue disease, overlap syndromes involving myositis, or in some cases of myositis alone. This antibody reacts with proteins that are uniquely present on the U1 small nuclear ribonucleoprotein, which is one of the nuclear RNPs that are involved in splicing mRNA. Autoantibodies such as anti-Sm, anti-Ro/SSA, and anti-La/SSB, that are usually associated with other conditions, are sometimes found in patients with overlap syndromes. Anti-Ku has been found in myositis-scleroderma overlap syndrome and in SLE. The Ku antigen is a DNA binding protein complex with two polypeptide components, both of which have been cloned.

Anti Jo-1 and other anti-synthetases are disease specific. Other myositis-associated antibodies are anti-PM-Scl, which is present in about 5-10% of myositis patients, many of whom have polymyositis-scleroderma overlap, and anti-Mi-2, which is present in about 8% of myositis patients, almost exclusively in dermatomyositis. Mi-2 is found in high titer in about 20% of all dermatomyositis patients and in low titer in less than 5% of polymyositis patients (see, e.g., Targoff and Reichlin, Mt. Sinai J. of Med. 55: 487-493 (1988)).

Anti-Mi was first described by Reichlin and Mattioli, Clin. Immunol. and Immunopathol. 5: 12-20 (1976)). A complement-fixation reaction was used to detect it and, in that study, patients with dermatomyositis, polymyositis and polymyositis overlap syndromes had positive reactions. The prototype or reference serum, from patient Mi, forms two precipitin lines on immunodiffusion (ID) with calf thymus antigens, Mi-1 and Mi-2. Mi-1, which has been purified from bovine thymus nuclear extracts (Nishikai, et al. Mol. Immunol. 17: 1129-141 (1980)) is rarely found in other sera and is not myositis specific (Targoff, et al., Clin. Exp. Immunol. 53: 76-82 (1983)).

Anti-Mi-2 was found to be a myositis-specific autoantibody by Targoff, et al. Arthritis and Rheum. 28: 796-803 (1985). Furthermore, all patients with the antibody have the dermatomyositis rash.

Bovine thymus Mi-2 antigen was originally found to be a nuclear protein that separates in SDS polyacrylamide (SDS-PAGE) gels into two bands with apparent molecular weights of 53 kilodaltons (hereinafter kDa) and 61 KDa, respectively. Recently, additional higher molecular weight bands have been found. The bovine thymus antigenic activity is destroyed by SDS-PAGE and is trypsin sensitive, but not RNAse sensitive (Targroff et al. Arthritis and Rheum. 28: 796-803 (1985)).

Anti-PM-1 was first identified as an antibody found in 61% of dermatomyositis/polymyositis patients, including patients; with polymyositis-scleroderma overlap (Wolfe, et al. J. Clin. Invest. 59: 176-178 (1977)). PM-1 was subsequently shown to be more than one antibody. The unique specificity component of PM-1 was later named PM-Scl (Reichlin, et al. J. Clin. Immunol. 4: 40-44 (1984)). Anti-PM-Scl is found in the sera of about 5-10% of myositis patients, but is most commonly associated with polymyositis-scleroderma overlap syndrome. It also occurs in patients with polymyositis or dermatomyositis alone or in patients with scleroderma without myositis.

Anti-PM-Scl antibody immunoprecipitates a complex from HeLa cell extracts of at least eleven polypeptides that have molecular weights ranging from about 20 to 110 kDa (see, Reimer, et al., J. Immunol. 137:3802-3808 (1986). The antigen is trypsin-sensitive, occurs in nucleoli (see, e.g., Targoff and Reichlin Arthritis Rheum. 28: 226-230 (1985)) and is believed to be a preribosomal particle.

In an abstract, Bluthner, et al., First Int. Workshop on the Mol. and Cell Biology of Autoantibodies and Autoimmunity in Heidelberg (Springer-Verlag Jul. 27-29, 1989) report that sera from patients suffering from polymyositis/scleroderma-overlap syndrome (PM/Scl) recognize two major nucleolar proteins of 95 and 75 kDa molecular weight in Western blots of a Hela cell extract. They also report that cDNA that encodes a 20 kDa protein reactive with autoantibodies eluting from the 95 kDa PM-Scl HeLa antigen subunit has been cloned from a HeLa cDNA library. The sequence of the cloned DNA has not as yet been reported.

It will be appreciated that combinations of myositis/scleroderma autoimmune/bystander antigens and myositis/scleroderma autoimmune/bystander antigenic determinants and/or polynucleotide sequences coding for them may also be used as appropriate.

Examples of myositis/scleroderma autoantigens and myositis/scleroderma bystander antigens include, but are not limited to, Jo-1 (his-tRNA synthetase), PM-Scl, Mi-2, Ku, PL-7 (thr-tRNA synthetase), PL-12 (ala-tRNA-synthetase), SRP (signal recognition particle), Anti-nRNP (U1 small nuclear RNP), Ro/SS-A, and La/SS-B.

For example, an amino acid sequence for a human 100 kD Pm-Scl autoantigen protein (PM/Scl-100a) is reported as follows (GenBank Accession No L01457; SEQ ID NO:37):

MAPPSTREPRVLSATSATKSDGEMVLPGFPDADSFVKFALGSVVAVTKAS
GGLPQFGDEYDFYRSFPGFQAFCETQGDRLLQCMSRVMQYHGCRSNIKDR
SKVTELEDKFDLLVDANDVILERVGILLDEASGVNKNQQPVLPAGLQVPK
TVVSSWNRKAAEYGKKAKSETFRLLHAKNIIRPQLKFREKIDNSNTPFLP
KIFIKPNAQKPLPQALSKERRERPQDRPEDLDVPPALADFIHQQRTQQVE
QDMFAHPYQYELNHFTPADAVLQKPQPQLYRPIEETPCHFISSLDELVEL
NEKLLNCQEFAVDLEHHSYRSFLGLTCLMQISTRTEDFIIDTLELRSDMY
ILNESLTDPAIVKVFHGADSDIEWLQKDFGLYVVNMFDTHQAARLLNLGR
HSLDHLLKLYCNVDSNKQYQLADWRIRPLPEEMLSYARDDTHYLLYIYDK
MRLEMWERGNGQPVQLQVVWQRSRDICLKKFIKPIFTDESYLELYRKQKK
HLNTQQLTAFQLLFAWRDKTARREDESYGYVLPNHMMLKIAEELPKEPQG
IIACCNPVPPLVRQQINEMHLLIQQAREMPLLKSEVAAGVKKSGPLPSAE
RLENVLFGPHDCSHAPPDGYPIIPTSGSVPVQKQASLFPDEKEDNLLGTT
CLIATAVITLFNEPSAEDSKKGPLTVAQKKAQNIMESFENPFRMISNRWK
LAQVQVQKDSKEAVKKKAAEQTAAREQAKEACKAAAEQAISVRQQVVLEN
AAKKRERATSDPRTTEQKQEKKRLKISKKPKDPEPPEKEFTPYDYSQSDF
KAFAGNSKSKVSSQFDPNKQTPSGKKCIAAKKIKQSVGNKSMSFPTGKSD
RGFRYNWPQR

(See also Gee et al., Cloning of a complementary DNA coding for the 100-kD antigenic protein of the PM-Scl autoantigen, J. Clin. Invest. 90 (2), 559-570 (1992).)

An amino acid sequence for a human 100 kD Pm-Scl autoantigen protein (PM/Scl-100b) is reported as follows (GenBank Accession No X66113; SEQ ID NO:38):

MAPPSTREPRVLSATSATKSDGEMVLPGFPDADSFVKFALGSVVAVTKAS
GGLPQFGDEYDFYRSFPGFQAFCETQGDRLLQCMSRVMQYHGCRSNIKDR
SKVTELEDKFDLLVDANDVILERVGILLDEASGVNKNQQPVLPAGLQVPK
TVVSSWNRKAAEYGKKAKSETFRLLHAKNIIRPQLKFREKIDNSNTPFLP
KIFTKPNAQKPLPQALSKERRERPQDRPEDLDVPPALADFIHQQRTQQVE
QDMFAHPYQYELNHFTPADAVLQKPQPQLYRPIEETPCHFISSLDELVEL
NEKLLNCQEFAVDLEHHSYRSFLGLTCLMQISTRTEDFIIDTLELRSDMY
ILNESLTDPAIVKVFHGADSDIEWLQKDFGLYVVNMFDTHQAARLLWLGR
HSLDHLLKLYCNVDSNKQYQLADWRIRPLPEEMLSYARDDTHYLLYIYDK
MRLEMWERGNGQPVQLQVVWQRSRDICLKKFIKPIFTDESYLELYRKQKK
HLNTQQLTAFQLLFAWRDKTARREDESYGYVLPNHMMLKIAEELPKEPQG
IIACCNPVPPLVRQQINEMHLLIQQAREMPLLKSEVAAGVKKSGPLPSAE
RLENVLFGPHDCSHAPPDGYPIIPTSGSVPVQKQASLFPDEKEDNLLGTT
CLIATAVITLFNEPSAEDSKKGPLTVAQKKAQNIMESFENPFRMFLPSLG
HRAPVSQAAKFDPSTKIYEISNRWKLAQVQVQKDSKEAVKKKAAEQTAAR
EQAKEACKAAAEQAISVRQQVVLENAAKKREPATSDPRTTEQKQEKKRLK
ISKKPKDPEPPEKEFTPYDYSQSDFKAFAGNSKSKVSSQFDPNKQTPSGK
KCIAAKKIKQSVGNKSMSFPTGKSDRGFRYNWPQR

(See also Bluthner and Bautz, Cloning and characterization of the cDNA coding for a polymyositis-scleroderma overlap syndrome-related nucleolar 100-kD protein, J. Exp. Med. 176 (4), 973-980 (1992).)

An amino acid sequence for a human75 kD Pm-Scl autoantigen protein (PM/Scl-75a) is reported as follows (GenBank Accession No M58460; SEQ ID NO:39):

MAAPAFEPGRQSDLLVKLNRLMERCLRNSKCIDTESLCVVAGEKVWQIRV
DLHLLNHDGNIIDAASIAAIVALCHFRRPDVSVQGDEVTLYTPEERDPVP
LSIHHMPICVSFAFFQQGTYLLVDPMEREERVMDGLLVIAMNKHREICTI
QSSGGIMLLKDQVLRCSKIAGVKVAEITELILKALENDQKVRKEGGKFGF
AESIANQRITAFKMEKAPIDTSDVEEKAEEIIAEAEPPSEVVSTPVLWTP
GTAQIGEGVENSWGDLEDSEKEDDEGGGDQAIILDGIKMDTGVEVSDIGS
QDAPIILSDSEEEEMIILEPDKNPKKIRTQTTSAKQEKAPSKKPVKRRKK
KRAAN

(See also Alderuccio et al., Molecular characterization of an autoantigen of PM-Scl in the polymyositis/scleroderma overlap syndrome: a unique and complete human cDNA encoding an apparent 75-kD acidic protein of the nucleolar complex, J. Exp. Med. 173 (4), 941-952 (1991).)

An amino acid sequence for a human 75 kD Pm-Scl autoantigen protein (PM/Scl-75b) is reported as follows (GenBank Accession No U09215; SEQ ID NO:40):

MAAPAFEPGRQSDLLVKLNRLMERCLRNSKCIDTESLCVVAGEKVWQIRV
DLHLLNHDGNIIDAASIAAIVALCHFRRPDVSVQGDEVTLYTPEERDPVP
LSIHHMPICVSFAFFQQGTYLLVDPNEREERVMDGLLVIAMNKHREICTI
QSSGGIMLLKDQVLRCSKIAGVKVAEITELILKALENDQKVRKEGGKFGF
AESIANQRITAFKMEKAPIDTSDVEEKAEEIIAEAEPPSEVVSTPVLWTP
GTAQIGEGVENSWGDLEDSEKEDDEGGGDQAIILDGIKMDTGVEVSDIGS
QELGFHHVGQTGLEFLTSDAPIILSDSEEEEMIILEPDKNPKKIRTQTTS
AKQEKAPSKKPVKRRKKKRAAN

An amino acid sequence for a Jo-1 (histidyl-tRNA synthetase) autoantigen protein is reported as follows (GenBank Accession No Z11518; SEQ ID NO:41):

MAERAALEELVKLQGERVRGLKQQKASAELIEEEVAKLLKLKAQLGP
DESKQKFVLKTPKGTRDYSPRQMAVREKVFDVIIRCFKRHGAEVIDTPVP
ELKETLMGKYGEDSKLIYDLKDQGGELLSLRYDLTVPFARYLAMNKLTNI
KRYHIAKVYRRDNPAMTRGRYREFYQCDFDIAGNFDPMIPDAECLKIMCE
ILSSLQIGDFLVKVNDRRILDGMFAICGVSDSKFRTICSSVDKLDKVSWE
EVKNEMVGEKGLAPEVADRIGDYVQQHGGVSLVEQLLQDPKLSQNKQALE
GLGDLKLLFEYLTLFGIDDKISFDLSLARGLDYYYGVIYEAVLLQTPAQA
GEEPLGVGSVAAGGRYDGLVGMFDPKGRKVPCVGLSIGVERIFSIVEQRL
EAELEEKIRTTETQVLVASAQKKLLEERLKLVSELWDAGIKAELLYKKNP
KLLNQLQYCEEAGIPLVAIIGEQELKDGVIKLRSVTSREEVDVRREDLVE
EIKRRTGQPLCIC

(See also Raben et al., Human histidyl-tRNA synthetase: recognition of amino acid signature regions in class 2a aminoacyl-tRNA synthetases, Nucleic Acids Res. 20 (5), 1075-1081 (1992).)

An amino acid sequence for a PL-7 (threonyl-tRNA synthetase) autoantigen protein is reported as follows (GenBank Accession No M63180; SEQ ID NO:42):

MGGEEKPIGAGEEKQKEGGKKKNKEGSGDGGRAELNPWPEYIYTRLEMYN
ILKAEHDSILAEKAEKDSKPIKVTLPDGKQVDAESWKTTPYQIACGISQG
LADNTVIAKVNNVVWDLDRPLEEDCTLELLKFEDEEAQAVYWHSSAHIMG
EGMERVYGGCLCYGPPIENGFYYDMYLEEGGVSSNDFSSLEALCKKIIKE
KQAFERLEVKKETLLANFKYNKFKCRILNEKVNTPTTTVYRCGPLIDLCR
GPHVRHTGKIKALKIHKNSSTYWEGKADMETLQRIYGISFPDPKMLKEWE
KFQEEAKNRDHRKIGRDQELYFFHELSPGSCFFLPKGVYIYNALIEFIRS
EYRKRGFQEVVTPNIFNSRLWMTSGHWQHYSENMFSFEVEKELFALKPNN
CPGHSLMFDHRPRSWRELPLRLADFGGLHRNELSGALTGLTRVRRFQQDD
AHIFCAMEQIEDEIKGCLDFLRTVYSVFGFSFKLNLSTRPEKFLGDIEVW
DQAEKQLENSLNEFGEKWELNSGDGAFYGPKIDIQIKDAIGRYHQCATIQ
LDFQLPIRFNLTYVSHDGEDKKRPVIVHPAILGSVERMIAILTENYGGKL
APFWLSPRQVMVVPVGPTCDEYAQNVRQQFHDAKFMADIDLDPGCTLNKK
IRNAQLAQYNFILVVGEKEKITGTVNIRTRDNKVHGERTISETIERLQQL
KEFRSKQAEEEF

(See also Cruzen et al., Nucleotide and deduced amino acid sequence of human threonyl-tRNA synthetase reveals extensive homology to the Escherichia coli and yeast enzymes, J. Biol. Chem. 266 (15), 9919-9923 (1991).)

An amino acid sequence for a PL-12 (alanyl-tRNA synthetase) autoantigen protein is reported as follows (GenBank Accession No D32050; SEQ ID NO:43):

MDSTLTASEIRQRFIDFFKRNEHTYVHSSATIPLDDPTLLFANAGMNQFK
PIFLNTIDPSHPMAKLSRAANTQKCIRAGGKQNDLDDVGKDVYHHTFFEM
LGSWSFGDYFKELACKMALELLTQEFGIPIERLYVTYFGGDEAAGLEADL
ECKQIWQNLGLDDTKILPGNMKDNFWEMGDTGPCGPCSEIHYDRIGGRDA
AHLVNQDDPNVLEIWNLVFIQYNREADGILKPLPKKSIDTGMGLERLVSV
LQNKMSNYDTDLFVPYFEAIQKGTGARPYTGKVGAEDADGIDMAYRVLAD
HARTITVALADGGRPDNTGRGYVLRRILRRAVRYAHEKLNASRGFFATLV
DVVVQSLGDAFPELKKDPDMVKDIINEEEVQFLKTLSRGRRILDRKIQSL
GDSKTIPGDTAWLLYDTYGFPVDLTGLIAEEKGLVVDMDGFEEERKLAQL
KSQGKGAGGEDLIMLDIYAIEELRARGLEVTDDSPKYNYHLDSSGSYVFE
NTVATVMALRREKMFVEEVSTGQECGVVLDKTCFYAEQGGQIYDEGYLVK
VDDSSEDKTEFTVKNAQVRGGYVLHTGTIYGDLKVGDQVWLFIDEPRRRP
IMSNHTATHILNFALRSVLGEADQKGSLVAPDRLRFDFTAKGANSTQQIK
KAEEIANEMIEAAKAVYTQDCPLAAAKAIQGLRAVFDETYPDPVRVVSIG
VPVSELLDDPSGPAGSLTSVEFCGGTHLRNSSHAGAFVIVTEEAIAKGIR
RIVAVTGAEAQKALRKAESLKKCLSVMEAKVKAQTAPNKDVQREIADLGE
ALATAVIPQWQKDELRETLKSLKKVMDDLDRASKADVQKRVLEKTKQFID
SNPNQPLVILEMESGASAKALNEALKLFKMHSPQTSAMLFTVDNEAGKIT
CLCQVPQNAANRGLKASEWVQQVSGLMDGKGGGKDVSAQATGKNVGCLQE
ALQLATSFAQLRLGDVKN

An amino acid sequence for an EJ (glycyl-tRNA synthetase) autoantigen protein is reported as follows (GenBank Accession No U09587; SEQ ID NO:44):

MDGAGAEEVLAPLRLAVRQQGDLVRKLKEDKAPQVDVDKAVAELKARKRV
LEAKELALQPKDDIVDRAKMEDTLKRRFFYDQAFAIYGGVSGLYDFGPVG
CALKNNIIQTWRQHFIQEEQILEIDCTMLTPEPVLKTSGHVDKFADFMVK
DVKNGECFRADHLLKAHLQKLMSDKKCSVEKKSEMESVLAQLDNYGQQEL
ADLFVNYNVKSPITGNDLSPPVSFNLMFKTFIGPGGNMPGYLRPETAQGI
FLNFKRLLEFNQGKLPFAAAQIGWSFRNEISPRSGLIRVREFTMAEIEHF
VDPSEKDHPKFQNVADLHLYLYSAKAQVSGQSARKMRLGDAVEQGVINNT
VLGYFIGRIYLYLTKVGISPDKLRFRQHMENEMAHYACDCWDAESKTSYG
WIEIVGCADRSCYDLSCHARATKVPLVAEKPLKEPKTVNVVQFEPSKGAI
GKAYKKDAKLVMEYLAICDECYITEMEMLLNEKGEFTIETEGKTFQLTKD
MINVKRFQKTLYVEEVVPNVIEPSFGLGRIMYTVFEHTFHVREGDEQRTF
FSFPAVVAPFKCSVLPLSQNQEFMPFVKELSEALTRHGVSHKVDDSSGSI
GRRYARTDEIGVAFGVTIDFDTVNKTPHTATLRDRDSMRQIRAEISELPS
IVQDLANGNITWADVEARYPLFEGQETGKKETIEE

Further sequences are provided, for example, under GenBank Accession Nos. AF241268.1, AF353396.1 (scleroderma-associated autoantigen); NM_{—}005033.1 (polymyositis/scleroderma autoantigen 1 (75 kDa) (PMSCL1)); XM_{—}001527.4, NM_{—}002685.1 (polymyositis/scleroderma autoantigen 2 (100 kDa) (PMSCL2)).

Nervous system Autoantigens and Bystander Antigens

In an alternative embodiment of the present invention the autoantigen or bystander antigen may be a nervous system autoantigen or bystander antigen for use to treat an autoimmune disease of the nervous system.

The term “autoimmune disease of the nervous system” includes any disease in which nervous tissue or a component thereof comes under autoimmune attack.

The term includes, for example central nervous system diseases having an autoimmune etiology such as multiple sclerosis (MS), perivenous encephalomyelitis, autoimmune myelopathies, paraneoplastic cerebellar degeneration, paraneoplastic limbic (cortical) degeneration, stiff man syndrome, choreas (such as Sydenham's chorea), stroke, focal epilepsy and migraine; and peripheral nervous system diseases having an autoimmune etiology such as Guillain-Barre syndrome, Miller Fisher syndrome, chronic inflammatory demyelinating neuropathy, multifocal motor neuropathy with conduction block, demyelinating neuropathy associated with anti-myelin-associated glycoprotein antibodies, paraneoplastyic sensory neuropathy, POEMS, dorsal root ganglion neuronitis, acute panautonomic neuropathy and brachial neutritis.

The term “nervous system autoantigen” as used herein includes any nervous system substance or a component thereof normally found within a mammal that, in an autoimmune disease of the nervous system, becomes a target of attack by the immune system, preferably the primary (or a primary) target of attack. The term also includes antigenic substances that induce conditions having the characteristics of an autoimmune disease of the nervous system when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “nervous system bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of the nervous system. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Preferably the nervous system autoantigen or nervous system bystander antigen is an MS autoantigen or MS bystander antigen.

The term “MS autoantigen” as used herein includes any nervous system substance or a component thereof normally found within a mammal that, in multiple sclerosis (MS), becomes a target of attack by the immune system, preferably the primary (or a primary) target of attack. The term also includes antigenic substances that induce conditions having the characteristics of MS when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “MS bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of nervous tissue under autoimmune attack in MS. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of nervous system autoimmune/bystander antigens and nervous system autoimmune/bystander antigenic determinants and/or polynucleotide sequences coding for them may also be used as appropriate.

Examples of nervous system autoantigens and nervous system bystander antigens include, but are not limited to, myelin basic proteins (MBPs), DM20,central nervous system white matter; proteolipid proteins (PLPs); myelin oligodendrocyte-associated proteins (MOGs), myelin associated glycoproteins (MAGs), alpha B-crystallins (see e.g. J. Chromatog. Biomed. Appl. 526:535 (90))

The protein components of myelin proteins, including myelin basic protein (MBP) I proteolipid protein (PLP), myelin-associated glycoprotein (MAG) and myelin oligodendrocyte glycoprotein (MOG), are of particular interest. The suppression of T cell responsiveness to these antigens may be used to prevent or treat demyelinating diseases.

Proteolipid is a major constituent of myelin, and is known to be involved in demyelinating diseases (see, for example Greer et al. (1992) J. Immunol. 149: 783-788 and Nicholson (1997) Proc. Natl. Acad. Sci. USA 94: 9279-9284).

The integral membrane protein PLP is a dominant autoantigen of myelin.

Determinants of PLP antigenicity have been identified in several mouse strains, and includes residues 139-151 (Tuohy et al. (1989) J. Immunol. 142: 1523-1527), residues 103-116 (Tuohy et al. (1988) J. Immunol. 141: 1126-1130), residues 215-232 (Endoh et al. (1990) Int. Arch. Allerqv Appl. Immunol. 92: 433-438), residues 43-64 (Whitham et al. (1991) J. Immunol. 147: 3803-3808) and residues 178-191 (Greer, et al. (1992) J. Immunol. 149: 783-788). Immunization with native PLP or with synthetic peptides corresponding to PLP epitopes induces experimental allergic encephalomyelitis (EAE). Analogues of PLP peptides generated by amino acid substitution can prevent EAE induction and progression (Kuchroo et al. (1994) J. Immunol. 153: 3326-3336, Nicholson et al. (1997) Proc. Natal. Acad. Sci. USA 94:9279-9284).

An amino acid sequence for a human proteolipid protein is reported as follows (GenBank Accession No M27110; SEQ ID NO:45):

MGLLECCARCLVGAPFASLVATGLCFFGVALFCGCGHEALTGTEKLIETY
FSKNYQDYEYLINVIHAFQYVIYGTASFFFLYGALLLAEGFYTTGAVRQI
FGDYKTTICGKGLSATVTGGQKGRGSRGQHQAHSLERVCTCLGKWLGHPD
KFVGITYALTVVWLLVFACSAVPVYIYFNTWTTCQSIAFPSKTSASIGSL
CADARMYGVLPWNAFPGKVCGSNLLSICKTAEFQMTFHLFIAAFVGAAAT
LVSLLTFMIAATYNFAVLKLMGRGTKF

MBP is an extrinsic myelin protein that has been studied extensively. At least 26 MBP epitopes have been reported (Meinl et al. (1993) J. Clin. Invest. 92: 2633-2643). Of particular interest are residues 1-11, 59-76 and 87-99. Analogues of MBP peptides generated by truncation have been shown to reverse EAE (Karin et al. (1998) J. Immunol. 160: 5188-5194). DNA encoding polypeptide fragments may comprise coding sequences for immunogenic epitopes, e.g. myelin basic protein p84-102, more particularly myelin basic protein p87-99, VHFFKNIVTPRTP (p87-99; SEQ ID NO:104), or the truncated 7-mer peptide FKNIVTP (SEQ ID NO:105). The sequences of myelin basic protein exon 2, including the immunodominant epitope bordered by amino acids 59-85, are also of interest. For examples, see Sakai et al. (1988) J Neuroimmunol 19: 21-32; Baxevanis et al. (1989) J Neuroimmunol 22: 23-30; Ota et al. (1990) Nature 346: 183-187; Martin et al. (1992) J Immunol. 148: 1350-1366, Valli et al. (1993) J Clin In 91: 616. The immunodominant MBP (84102) peptide has been found to bind with high affinity to DRB1*1501 and DRB5*0101 molecules of the disease-associated DR2 haplotype. Overlapping but distinct peptide segments were important for binding to these molecules; hydrophobic residues (Val189 and Phe92) in the MBP (88-95) segment for peptide binding to DRB1*1501 molecules; hydrophobic and charged residues (Phe92, Lys93) in the MBP (89-101/102) sequence contributed to DRB5*0101 binding.

An amino acid sequence for a human myelin basic protein (MBP) is reported as follows (GenBank Accession No M13577; SEQ ID NO:46):

MASQKRPSQRHGSKYLATASTMDHARHGFLPRHRDTGILDSIGRFFGGDR
GAPKRGSGKDSHHPARTAHYGSLPQKSHGRTQDENPVVHFFKNIVTPRTP
PPSQGKGRGLSLSRFSWGAEGQRPGFGYGGPASDYKSAHKGFKGVDAQGT
LSKIFKLGGRDSRSGSPMARR

The transmembrane glycoprotein MOG is a minor component of myelin that has been shown to induce EAE. Immunodominant MOG epitopes that have been identified in several mouse strains include residues 1-22,35-55,64-96 (deRosbo et al. (1998) J. Autoimmunity 11: 287-299, deRosbo ef al. (1995) Eur J Immunol. 25: 985-993) and 41-60 (Leadbetter et al. (1998) J Immunol 161: 504-512).

An amino acid sequence for a human myelin/oligodendrocyte glycoprotein (MOG) protein (25.1 kD) is reported as follows (GenBank Accession No U64564; SEQ ID NO:47):

MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLVFLCLQYRLRGKLRAEIENLHRTFDPH
FLRVPCWKITLFVIVPVLGPLVALIICYNWLHRRLAGQFLEELRNPF

An amino acid sequence for a human myelin-associated glycoprotein (MAG) is reported as follows (GenBank Accession No M29273; SEQ ID NO:48):

MIFLTALPLFWIMISASRGGHWGAWMPSSISAFEGTCVSIPCRFDFPDEL
RPAVVHGVWYFNSPYPKNYPPVVFKSRTQVVHESFQGRSRLLGDLGLRNC
TLLLSNVSPELGGKYYFRGDLGGYNQYTFSEHSVLDIVNTPNIVVPPEVV
AGTEVEVSCMVPDNCPELRPELSWLGHEGLGEPAVLGRLREDEGTWVQVS
LLHFVPTREANGHRLGCQASFPNTTLQFEGYASMDVKYPPVIVEMNSSVE
AIEGSHVSLLCGADSNPPPLLTWMRDGTVLREAVAESLLLELEEVTPAED
GVYACLAENAYGQDNRTVGLSVMYAPWKPTVNGTMVAVEGETVSILCSTQ
SNPDPILTIFKEKQILSTVIYESELQLELPAVSPEDDGEYWCVAENQYGQ
RATAFNLSVEFAPVLLLESHCAAARDTVQCLCVVKSNPEPSVAFELPSRN
VTVNESEREFVYSERSGLVLTSILTLRGQAQAPPRVICTARNLYGAKSLE
LPFQGAHRLMWAKIGPVGAVVAFATLIAIVCYITQTRRKKNVTESPSFSA
GDNPPVLFSSDFRISGAPEKYESERRLGSERRLLGLRGEPPELDLSYSHS
DLGKRPTKDSYTLTEELAEYAEIRVK

In one embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | | |
|---|---|---|
| GRTQDENPVVHFFKNIVTPRTPP | MBP (aa 80-102) | (SEQ ID NO:49) |
| AVYVYIYFNTWTTCQFIAFPFK | MBP (aa 170-191) | (SEQ ID NO:50) |
| SQRHGSKYLATASTMDHARHG | MBP (aa 7-27) | (SEQ ID NO:51) |
| RDTGILDSIGRFFGGDRGAP | MBP (aa 33-52) | (SEQ ID NO:52) |
| QKSHGRTQDENPVVEFFKNI | MBP (aa 74-93) | (SEQ ID NO:53) |
| DENPVVHFFKNIVT | MBP (aa 84-97) | (SEQ ID NO:54) |
| ENPVVHFFKNIVTPR | MBP (aa 85-99) | (SEQ ID NO:55) |
| HFFKNTVTPRTPP | MBP (aa 90-102) | (SEQ ID NO:56) |
| KGFKGVDAQGTLSK | MBP (aa 139-152) | (SEQ ID NO:57) |
| VDAQGTLSKIFKLGGRDSRS | MBP (aa 144-163) | (SEQ ID NO:58) |

Autoimmune Arthritis Autoantigens and Bystander Antigens

In an alternative embodiment of the present invention the autoantigen or bystander antigen may be autoimmune arthritis autoantigen or bystander antigen for use to treat autoimmune arthritis.

The term “autoimmune arthritis autoantigen” as used herein includes any substance or a component thereof normally found within a mammal that, in autoimmune arthritis (especially rheumatoid arthritis (RA)), becomes a target of attack by the immune system, preferably the primary (or a primary) target of attack. The term also includes antigenic substances that induce conditions having the characteristics of autoimmune arthritis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “autoimmune arthritis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in autoimmune arthritis, especially rheumatoid arthritis (RA). The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

The term “autoimmune arthritis” includes rheumatoid arthritis, juvenile arthritis, psoriatic arthritis, spondylo arthritis, relapsing polychondritis and other connective tissue diseases having an autoimmune disease component.

It will be appreciated that combinations of RA autoimmune/bystander antigens and RA autoimmune/bystander antigenic determinants and/or polynucleotide sequences coding for them may also be used as appropriate.

Some examples of RA autoantigens and RA bystander antigens include, but are not limited to, antigens from connective tissue, collagen (especially types I, II, III, IX, and XI), heat shock proteins and immunoglobulin Fc domains (see, e.g. J. Immunol. Methods 121:21 9 (89) and 151:177 (92)).

Collagen is a family of fibrous proteins that have been classified into a number of structurally and genetically distinct types (Stryer, L. Biochemistry, 2nd Edition, W. H. Freeman & Co., 1981, pp. 184-199). Type I collagen is the most prevalent form and is found inter alia, in skin, tendons, cornea and bones and consists of two subunits of alpha1(I) collagen and one subunit of a different sequence termed alpha2. Other types of collagen, including type II collagen, have three identical subunits or chains, each consisting of about 1,000 amino acids. Type II collagen (“CII”) is the type of collagen found inter alia, in cartilage, the interverbebral disc and the vitreous body. Type II collagen contains three alpha1(II) chains (alpha1(II)₃). Type III collagen is found inter alia, in blood vessels, the cardiovascular system and fetal skin and contains three alpha1(III) chains (alpha1(III)₃). Type IV collagen is localized, inter alia, in basement membranes and contains three alpha 1 (IV) chains (alpha1(IV)₃).

Diabetes Autoantigens and Bystander Antigens

In an alternative embodiment of the present invention the autoantigen or bystander antigen may be a diabetes autoantigen or bystander antigen for use to treat autoimmune diabetes.

The term “autoimmune diabetes” as used herein includes all forms of diabetes having an autoimmune component, and, in particular, Type I diabetes (also known as juvenile diabetes or insulin-dependent diabetes mellitus; IDDM). Type I diabetes is a disease that affects mainly children and young adults. The clinical features of the disease are caused by an insufficiency in the body's own insulin production due to a significant or even total reduction in of insulin production. It has been found that this type of diabetes is an autoimmune disease (cf. Castano, L. and G. S. Eisenbirth (1990) Type I diabetes: A chronic autoimmune disease of human, mouse and rat. Annu. Rev. Immunol. 8:647-679).

All cells of the immune system play a more or less important role. The B lymphocytes produce autoantibodies, whereas the monocytes/macrophages are probably involved in the induction of autoimmunity as antigen presenting cells. It is understood that T lymphocytes play a major role as effector cells in the destruction reaction. Like most autoimmune diseases type I diabetes arises because the tolerance of the T cells towards the body's own tissue (“self”) is lost. In particular, loss of tolerance towards pancreatic beta cells will result in the destruction thereof and diabetes will arise.

It is reported that about 30% to 40% of diabetic children will eventually develop nephropathy requiring dialysis and transplantation (see U.S. Pat. No. 5,624,895) Other significant complications include cardiovascular disease, stroke, blindness and gangrene. Moreover, diabetes mellitus accounts for a significant proportion of morbidity and mortality among dialysis and transplant patients.

Onset of Type I diabetes mellitus normally results from a well-characterized insulitis. During this condition, the inflammatory cells are typically directed against the beta cells of the pancreatic islets. It has been demonstrated that a large proportion of the infiltrating T lymphocytes produced during Type I diabetes mellitus are CD8-positive cytotoxic cells, which confirms the cytotoxic activity of the cellular infiltrate. CD4-positive lymphocytes are also present, the majority of which are helper T cells (Bottazzo et at., 1985, New England Journal of Medicine, 313, 353-359). The infiltrating cells also include lymphocytes or B cells that produce immunoglobulin-G (IgG) which suggest that these antibody-producing cells infiltrate the pancreatic islets (Glerchmann et at., 1987, Immunology Today, 8, 167-170).

The term “diabetes autoantigen” as used herein includes any substance or a component thereof normally found within a mammal that, in autoimmune diabetes, becomes a target of attack by the immune system, preferably the primary (or a primary) target of attack. The term also includes antigenic substances that induce conditions having the characteristics of autoimmune diabetes when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “diabetes bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue (usually the pancreas) under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of diabetes autoimmune/bystander antigens and diabetes autoimmune/bystander antigenic determinants and/or polynucleotide sequences coding for them may also be used as appropriate.

Examples of diabetes autoantigens and bystander antigens include, but are not limited to, pancreatic beta cell (Type I) antigens, insulins, insulin receptors, insulin associated antigens (IA-w), glucagons, amylins, gamma amino decarboxylases (GADs) and heat shock proteins (HSPs), carboxypeptidases, peripherins and gangliosides. Some of these are discussed in more detail below.

a) Preproinsulin

Human insulin mRNA is translated as a 110 amino acid single chain precursor called preproinsulin, and removal of its signal peptide during insertion into the endoplasmic reticulum generates proinsulin. Proinsulin consists of three domains: an amino-terminal B chain, a carboxy-terminal A chain and a connecting peptide in the middle known as the C peptide. Within the endoplasmic reticulum, proinsulin is exposed to several specific endopeptidases which excise the C peptide, thereby generating the mature form of insulin which consists of the A and B chain. Insulin and free C peptide are packaged in the Golgi into secretory granules which accumulate in the cytoplasm. The preproinsulin peptide sequence (SEQ ID NO:59) is reported as follows:

MALWMRLLPL LALLALWGPD PAAAFVNQHL CGSHLVEALY
LVCGERGFFY TPKTRREAED LQVGQVELGG GPGAGSLQPL
ALEGSLQKRG IVEQCCTSIC SLYQLENYCN

The insulin A chain includes amino acids 90-110 of this sequence. The B chain includes amino acids 25-54. The connecting sequence (amino acids 55-89) includes a pair of basic amino acids at either end. Proteolytic cleavage of proinsulin at these dibasic sequences liberates the insulin molecule and free C peptide, which includes amino acids 57-87. The human preproinsulin or an immunologically active fragment thereof, e.g., B chain or an immunogenic fragment thereof, e.g., amino acids 33-47 (corresponding to residues 9-23 of the B-chain), are useful as autoantigens in the methods and compositions described herein.

b) GAD65

Gad65 is a primary beta-cell antigen involved in the autoimmune response leading to insulin dependent diabetes mellitus (Christgau et al. (1991) J Biol Chem. 266 (31): 21257-64). The presence of autoantibodies to GAD65 is used as a method of diagnosis of type 1 diabetes. Gad65 is a 585 amino acid protein with a sequence (SEQ ID NO:60) reported as follows:

MASPGSGFWS FGSEDGSGDS ENPGTARAWC QVAQKFTGGI
GNKLCALLYG DAEKPAESGG SQPPRAAARK AACACDQKPC
SCSKVDVNYA FLHATDLLPA CDGERPTLAF LQDVMNILLQ
YVVKSFDRST KVIDFHYPNE LLQEYNWELA DQPQNLEEIL
MHCQTTLKYA IKTGHPRYFN QLSTGLDMVG LAADWLTSTA
NTNMFTYEIA PVFVLLEYVT LKKMREIIGW PGGSGDGIFS
PGGAISNMYA MMIARFKMFP EVKEKGMAAL PRLIAFTSEH
SHFSLKKGAA ALGIGTDSVI LIKCDERGKM IPSDLERRIL
EAKQKGFVPF LVSATAGTTV YGAFDPLLAV ADICKKYKIW
MHVDAAWGGG LLMSRKHKWK LSGVERANSV TWNPHKMMGV
PLQCSALLVR EEGLMQNCNQ MHASYLFQQD KHYDLSYDTG
DKALQCGRHV DVFKLWLMWR AKGTTGFEAH VDKCLELAEY
LYNIIKNREG YEMVFDGKPQ HTNVCFWYIP PSLRTLEDNE
ERMSRLSKVA PVIKARMMEY GTTMVSYQPL GDKVNFFRMV
ISNPAATHQD IDFLIEEIER LGQDL

c) Islet Tyrosine Phosphatase IA-2

IA-2/ICA512, a member of the protein tyrosine phosphatase family, is another major autoantigen in type 1 diabetes (Lan et al. DNA Cell Biol 13: 505-514, 1994).

It is reported that 70% of diabetic patients have autoantibodies to IA-2, which may appear years before the development of clinical disease. The IA-2 molecule is 979 amino acids in length and consists of an intracellular, transmembrane, and extracellular domain (Rabin et al. (1994) J. Immunol. 152 (6), 3183-3188). Autoantibodies are typically directed to the intracellular domain, e.g., amino acids 600-979 and fragments thereof (Zhang et al. (1997) Diabetes 46: 40-43 ; Xie et al. (1997) J Immunol 159: 3662-3667). The amino acid sequence of IA-2 (SEQ ID NO:61) is reported as follows:

MRRPRRPGGL GGSGGLRLLL CLLLLSSRPG GCSAVSARGC
LFDRRLCSHL EVCIQDGLFG QCQVGVGQAR PLLQVTSPVL
QRLQGVLRQL MSQGLSWHDD LTQYVISQEM ERIPRLRPPE
PRPRDRSGLA PKRPGPAGEL LLQDIPTGSA PAAQHRLPQP
PVGKGGAGAS SSLSPLQAEL LPPLLEHLLL PPQPPNPSLS
YEPALLQPYL FHQFGSRDGS RVSEGSPGMV SVGPLPKAEA
PALFSRTASK GIFGDHPGHS YGDLPGPSPA QLFQDSGLLY
LAQELPAPSR ARVPRLPEQG SSSRAEDSPE GYEKEGLGDR
GEKPASPAVQ PDAALQRLAA VLAGYGVELR QLTPEQLSTL
LTLLQLLPKG AGRNPGGVVN VGADIKKTME GPVEGRDTAE
LPARTSPMPG HPTASPTSSE VQQVPSPVSS EPPKAARPPV
TPVLLEKKSP LGQSQPTVAG QPSARPAAEE YGYIVTDQKP
LSLAAGVKLL EILAEHVHMS SGSFINISVV GPALTFRIRH
NEQNLSLADV TQQAGLVKSE LEAQTGLQIL QTGVGQREEA
AAVLPQTAHS TSPMRSVLLT LVALAGVAGL LVALAVALCV
RQHARQQDKE RLAALGPEGA HGDTTFEYQD LCRQHMATKS
LFNRAEGPPE PSRVSSVSSQ FSDAAQASPS SHSSTPSWCE
EPAQANMDIS TGHMILAYME DHLRNRDRLA KEWQALCAYQ
AEPNTCATAQ GEGNIKKNRH PDFLPYDHAR IKLKVESSPS
RSDYINASPI IEHDPRMPAY IATQGPLSHT IADFWQMVWE
SGCTVIVMLT PLVEDGVKQC DRYWPDEGAS LYHVYEVNLV
SEHIWCEDFL VRSFYLKNVQ TQETRTLTQF HFLSWPAEGT
PASTRPLLDF RRKVNKCYRG RSCPIIVHCS DGAGRTGTYI
LIDMVLNRMA KGVKEIDIAA TLEHVRDQRP GLVRSKDQFE
FALTAVAEEV NAILKALPQ

d) ICA12

ICA12 (Kasimiotis et al. (2000) Diabetes 49 (4): 555-61; GenBank Accession No. AAD16237) is one of a number of islet cell autoantigens associated with diabetes. The amino acid sequence of ICA12 (SEQ ID NO:62) is reported as follows:

MSMRSPISAQ LALDGVGTMV NCTIKSEEKK EPCHEAPQGS
ATAAEPQPGD PARASQDSAD PQAPAQGNFR GSWDCSSPEG
NGSPEPKRPG ASEAASGSQE KLDFNRNLKE VVPAIEKLLS
SDWKERFLGR NSMEAKDVKG TQESLAEKEL QLLVMIHQLS
TLRDQLLTAH SEQKNMAAML FEKQQQQMEL ARQQQEQIAK
QQQQLIQQQH KINLLQQQIQ QVNMPYVMIP AFPPSHQPLP
VTPDSQLALP IQPIPCKPVE YPLQLLHSPP APVVKRPGAM
ATHHPLQEPS QPLNLTAKPK APELPNTSSS PSLKMSSCVP
RPPSHGGPTR DLQSSPPSLP LGFLGEGDAV TKAIQDARQL
LHSHSGALDG SPNTPFRKDL ISLDSSPAKE RLEDGCVHPL
EEANLSCDMD GSRHFPESRN SSHIKRPMNA FMVWAKDERR
KILQAFPDMH NSSISKILGS RWKSMTNQEK QPYYEEQARL
SRQHLEKYPD YKYKPRPKRT CIVEGKRLRV GEYKALMRTR
RQDARQSYVI PPQAGQVQMS SSDVLYPRAA GMPLAQPLVE
HYVPRSLDPM MPVIVNTCSL REEGEGTDDR HSVADGEMYR
YSEDEDSEGE EKSDGELVVL TD

e) ICA69

ICA69 is another autoantigen associated with type 1 diabetes (Pietropaolo et al.

J Clin Invest 1993; 92: 359-371). An amino acid sequence of ICA69 (SEQ ID NO:63) is reported as follows:

MSGHKCSYPW DLQDRYAQDK SVVNKMQQRY WETKQAFIKA
TGKKEDEHVV ASDADLDAKL ELFHSIQRTC LDLSKAIVLY
QKRICFLSQE ENELGKFLRS QGFQDKTRAG KMMQATGKAL
CFSSQQRLAL RNPLCRFHQE VETFRHRAIS DTWLTVNRME
QCRTEYRGAL LWMKDVSQEL DPDLYKQMEK FRKVQTQVRL
AKKNFDKLKM DVCQKVDLLG ASRCNLLSHM LATYQTTLLH
FWEKTSHTMA AIHESFKGYQ PYEFTTLKSL QDPMKKLVEK
EEKKKINQQE STDAAVQEPS QLISLEEENQ RKESSSFKTE
DGKSILSALD KGSTHTACSG PIDELLDMKS EEGACLGPVA
GTPEPEGADK DDLLLLSEIF NASSLEEGEF SKEWAAVFGD
GQVKEPVPTM ALGEPDPKAQ TGSGFLPSQL LDQNMKDLQA
SLQEPAKAAS DLTAWFSLFA DLDPLSNPDA VGKTDKEHEL
LNA

f) Glima 38

Glima 38 is a 38 kDa islet cell membrane autoantigen which is specifically immunoprecipitated with sera from a subset of prediabetic individuals and newly diagnosed type 1 diabetic patients. Glima 38 is an amphiphilic membrane glycoprotein, specifically expressed in islet and neuronal cell lines, and thus shares the neuroendocrine expression patterns of GAD65 and IA2 (Aanstoot et al. J Clin Invest. 1996 Jun. 15; 97 (12): 2772-2783).

g) Heat Shock Protein 60 (HSP60)

HSP60, e.g., an immunologically active fragment of HSP60, e.g., p277 (see Elias et al., Eur J Immunol 1995 25 (10): 2851-7), can also be used as an autoantigen in the methods and compositions described herein. Other useful epitopes of HSP 60 are described, for example, in U.S. Pat. No. 6,110,746.

h) Carboxypeptidase H

Carboxypeptidase H has been identified as an autoantigen, e.g., in pre-type 1 diabetes patients (Castano et al. (1991) J Clin Endocrinol Metab 73 (6): 1197-201; Alcalde et al. J Autoimmun. 1996 August; 9 (4): 525-8.). Therefore, carboxypeptidase H or immunologically reactive fragments thereof (e.g., the 136-amino acid fragment of carboxypeptidase-H described in Castano, supra) can be used in the methods and compositions described herein.

i) Peripherin

Peripherin is a 58 KDa diabetes autoantigen identified in nod mice (Boitard et al. (1992) Proc Natl Acad Sci USA 89 (1): 172-6). A human peripherin sequence (SEQ ID NO:64) is reported as follows:

MSHHPSGLPA GFSSTSYRRT FGPPPSLSPG AFSYSSSSRF
SSSRLLGSAS PSSSVRLGSF RSPRAGAGAL LRLPSERLDF
SMAEALNQEF LATRSNEKQE LQELNDRFAN FTEKVRFLEQ
QNAALRGELS QARGQEPARA DQLCQQELRE LRRELELLGR
ERDRVQVERD GLAEDLAALK QRLEEETRKR EDAEHNLVLF
RKDVDDATLS RLELERKIES LMDEIEFLKK LHEEELRDLQ
VSVESQQVQQ VEVEATVKPE LTAALRDIRA QYESIAAKNL
QEAEEWYKSK YADLSDAANR NHEALRQAKQ EMNESRRQIQ
SLTCEVDGLR GTNEALLRQL RELEEQFALE AGGYQAGAAR
LEEELRQLKE EMARHLREYQ ELLNVKMALD IEIATYRKLL
EGEESRISVP VHSFASLNIK TTVPEVEPPQ DSHSRKTVLI
KTIETRNGEQ VVTESQKEQR SELDKSSAHS Y

j) Gangliosides

Gangliosides can also be useful autoantigens in the methods and compositions described herein. Gangliosides are sialic acid-containing glycolipids which are formed by a hydrophobic portion, the ceramide, and a hydrophilic part, i.e. the oligosaccharide chain. Gangliosides are expressed, inter alia, in cytosol membranes of secretory granules of pancreatic islets. Autoantibodies to gangliosides have been described in type 1 diabetes, e.g., GM1-2 ganglioside is an islet autoantigen in diabetes autoimmunity and is expressed by human native (3 cells (Dotta et al. Diabetes. 1996 September; 45 (9): 1193-6). Gangliosides GT3, GD3 and GM-1 are also the target of autoantibodies associated with autoimmune diabetes (reviewed in Dionisi et al. Aim Ist Super Sanita 1997; 33 (3): 433-5). Ganglioside GM3 participates in the pathological conditions of insulin resistance (Tagami et al. J Biol Chem 2001 Nov. 13; online publication ahead of print).

Further sequences are provided, for example, under GenBank Accession Nos U26593.1, BC008640.1, NM_{—}022308.1, NM_{—}022307.1, NM_{—}004968.1, AF146363.1, AF147807.1, AH008870.1, U37183.1, U38260.1, AH005787.1, U71264.1, U71263.1, U71262.1, U71261.1, U71260.1, U71259.1, U71258.1, U71257.1, U71256.1, U71255.1, U71254.1, U71253.1, U71252.1, U01882.1, U17989.1 (diabetes mellitus type I autoantigen (ICAp69)), X62899.2 (islet cell antigen 512), A28076.1 (islet GAD sequence (HIGAD-FL)) and AF098915.1 (type 1 diabetes autoantigen ICA12).

Myasthenia Gravis Autoantigens and Bystander Antigens

In an alternative embodiment of the present invention the autoantigen or bystander antigen may be a Myasthenia Gravis autoantigen or bystander antigen for use to treat Myasthenia Gravis.

The term “Myasthenia Gravis autoantigen” as used herein includes any substance or a component thereof normally found within a mammal that, in Myasthenia Gravis, becomes a target of attack by the immune system, preferably the primary (or a primary) target of attack. The term also includes antigenic substances that induce conditions having the characteristics of Myasthenia Gravis when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Myasthenia Gravis bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Myasthenia Gravis. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of Myasthenia Gravis autoimmune/bystander antigens and Myasthenia Gravis autoimmune/bystander antigenic determinants and/or polynucleotide sequences coding for them may also be used as appropriate.

Some examples of Myasthenia Gravis autoantigens and Myasthenia Gravis bystander antigens include, but are not limited to, acetyl choline receptors and components thereof, preferably human acetyl choline receptors and components thereof (see e.g. Eur. J. Pharm. 172:231(89)).

An amino acid sequence for a human gravin (A kinase (PRKA) anchor protein) autoantigen is reported as follows (GenBank Accession No M96322; SEQ ID NO:65):

DCQAKSTPVIVSATTKKGLSSDLEGEKTTSLKWKSDEVDEQVACQEVKVS
VAIEEDLEPENGILELETKSSKLVQNIIQTAVDQFVRTEETATEMLTSEL
QTQAHMIKADSQDAGQETEKEGEEPQASAQDETPITSAKEESESTAVGQA
HSDISKDMSEASEKTMTVEVEGSTVNDQQLEEVVLPSEEEGGGAGTKSVP
EDDGHALLAERIEKSLVEPKEDEKGDDVDDPENQNSALADTDASGGLTKE
SPDTNGPKQKEKEDAQEVELQEGKVHSESDKAITPQAQEELQKQERESAK
SELTES

An amino acid sequence for a human cholinergic receptor (gamma subunit) autoantigen is reported as follows (GenBank Accession No NM_{—}005199; SEQ ID NO:66):

MHGGQGPLLLLLLLAVCLGGTQRNLRNQEERLLADLMQNYDPNLRPAERD
SDVVNVSLKLTLTNLISLNEREEALTTNVWIEMQWCDYRLRWDPRDYEGL
WVLRVPSTMVWRPDIVLENNVDGVFEVALYCNVLVSPDGCIYWLPPAIFR
SACSISVTYFPFDWQNCSLIFQSQTYSTNEIDLQLSQEDGQTIEWIFIDP
EAFTENGEWAIQHRPAKMLLDPAAPAQEAGHQKVVFYLLIQRKPLFYVIN
IIAPCVLISSVAILIHFLPAKAGGQKCTVAINVLLAQTVFLFLVAKKVPE
TSQAVPLISKYLTFLLVVTILIVVNAVVVLNVSLRSPHTHSMARGVRKVF
LRLLPQLLRMHVRPLAPAAVQDTQSRLQNGSSGWSITTGEEVALCLPRSE
LLFQQWQRQGLVAAALEKLEKGPELGLSQFCGSLKQAAPAIQACVEACNL
IACARHQQSHFDNGNEEWFLVGRVLDDRVCFLAMLSLFICGTAGIFLMAH
YNRVPALPFPGDPRPYLPSPD

An amino acid sequence for a human cholinergic receptor (alpha subunit) autoantigen is reported as follows (GenBank Accession No S77094; SEQ ID NO:67):

MEPWPLLLLFSLCSAGLVLGSEHETRLVAKLFKDYSSVVRPVEDHRQVVE
VTVGLQLIQLINVDEVNQIVTTNVRLKQQWVDYNLKWNPDDYGGVKKIHI
PSEKIWRPDLVLYNNADGDFAIVKFTKVLLQYTGHITWTPPAIFKSYCEI
IVTHFPFDEQNCSMKLGTWTYDGSVVAINPESDQPDLSNFMESGEWVIKE
SRGWKHSVTYSCCPDTPYLDITYHFVMQRLPLYFIVNVIIPCLLFSFLTG
LVFYLPTDSGEKMTLSISVLLSLTVFLLVIVELIPSTSSAVPLIGKYMLF
TMVFVIASIIITVIVINTHHRSPSTHVMPNWVRKVFIDTIPNIMFFSTMK
RPSREKQDKKIFTEDIDISDISGKPGPPPMGFHSPLIKHFEVKSAIEGIK
YIAETMKSDQESNNAAAEWKYVANVMDHILLGVFMLVCIIGTLAVFAGRL
IELNQQG

(See also Gattenlohner et al, Cloning of a cDNA coding for the acetylcholine receptor alpha-subunit from a thymoma associated with myasthenia gravis, Thymus 23 (2), 103-113 (1994).)

Purified acetylcholine receptor can be isolated, for example, by the method of Mcintosh et al. J Neuroimmunol. 25: 75, 1989.

In an alternative embodiment one or more antigenic determinants may be used in place of a full antigen. For example, some specific class II MHC-associated autoantigen peptide sequences are as follows (see U.S. Pat. No. 5,783,567):

| | |
|---|---|
| Peptide Sequence | Source |

| | |
|---|---|
| TVGLQLIQLINVDEVNQIVTTNVRLKQQWVDYNLKW | AChR alpha (aa 32-67) (SEQ ID NO:68) |
| QIVTTNVRLKQQWVDYNLKW | AChR alpha (aa 48-67) (SEQ ID NO:69) |
| QWVDYNL | AChR alpha (aa 59-65) (SEQ ID NO:70) |
| GGVKKIHIPSEKIWRPDL | AChR alpha (aa 73-90) (SEQ ID NO:71) |
| AIVKFTKVLLQY | AChR alpha (aa 101-112) (SEQ ID NO:72) |
| WTPPAIFKSYCEIIVTHFPF | AChR alpha (aa 118-137) (SEQ ID NO:73) |
| MKLGTWTYDGSVV | AChR alpha (aa 144-156) (SEQ ID NO:74) |
| MKLGIWTYDGSVV | AChR alpha (aa 144-157) analog (I-148) (SEQ ID NO:75) |
| WTYDGSVVA | AChR alpha (aa 149-157) (SEQ ID NO:76) |
| SCCPDTPYLDITYHFVM | AChR alpha (aa 191-207) (SEQ ID NO:77) |
| DTPYLDITYHFVMQRLPL | AChR alpha (aa 195-212) (SEQ ID NO:78) |
| FIVNVIIPCLLFSFLTGLVFY | AChR alpha (aa 214-234) (SEQ ID NO:79) |
| LLVIVELIPSTSS | AChR alpha (aa 257-269) (SEQ ID NO:80) |
| STHVMPNWVRKVFIDTIPN | AChR alpha (aa 304-322) (SEQ ID NO:81) |
| NWVRKVFIDTIPNIMFFS | AChR alpha (aa 310-327) (SEQ ID NO:82) |
| IPNIMFFSTMKRPSREKQ | AChR alpha (aa 320-337) (SEQ ID NO:83) |
| AAAEWKYVAMVMDHIL | AChR alpha (aa 395-410) (SEQ ID NO:84) |
| IIGTLAVFAGRLIELNQQG | AChR alpha (aa 419-437) (SEQ ID NO:85) |
| GQTIEWIFIDPEAFTENGEW | AChR gamma (aa 165-184) (SEQ ID NO:86) |
| MAHYNRVPALPFPGDPRPYL | AChR gamma (aa 476-495) (SEQ ID NO:87) |

SLE Autoantigens and SLE Bystander Antigens

In an alternative embodiment of the present invention the autoantigen or bystander antigen may be a Systemic Lupus Erythematosus (SLE) autoantigen or bystander antigen for use to treat SLE.

The term “SLE autoantigen” as used herein includes any substance or a component thereof normally found within a mammal that, in Systemic Lupus Erythematosus (SLE), becomes a target of attack by the immune system, preferably the primary (or a primary) target of attack. The term also includes antigenic substances that induce conditions having the characteristics of an autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “SLE bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in SLE. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction, such as heatshock proteins (HSP), which although not necessarily specific to a particular tissue are normally shielded from the immune system.

It will be appreciated that combinations of SLE autoimmune/bystander antigens and SLE autoimmune/bystander antigenic determinants and/or polynucleotide sequences coding for them may also be used as appropriate.

Some examples of SLE autoantigens and SLE bystander antigens include, but are not limited to, ds-DNA, chromatins, histones, nucleolar antigens, soluble RNA protein particles (such as U1RNP, Sm, Ro/SSA and La/SSB) erythrocyte antigens and platelet antigens. Examples of proteins include, for example, the human Ku and La antigens.

For example, an amino acid sequence for a human lupus p70 (Ku) autoantigen protein is reported as follows (GenBank Accession No J04611; SEQ ID NO:88):

MSGWESYYKTEGDEEAEEEQEENLEASGDYKYSGRDSLIFLVDASKAMFE
SQSEDELTPFDMSIQCIQSVYISKIISSDRDLLAVVFYGTEKDKNSVNFK
NIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFL
DLMHLKKPGGFDISLFYRDIISIAEDEDLRVHFEESSKLEDLLRKVRAKE
TRKRALSRLKLKLNKDIVISVGIYNLVQKALKPPPIKLYRETNEPVKTKT
RTFNTSTGGLLLPSDTKRSQIYGSRQIILEKEETEELKRFDDPGLMLMGF
KPLVLLKKHHYLRPSLFVYPEESLVIGSSTLFSALLIKCLEKEVAALCRY
TPRRNIPPYFVALVPQEEELDDQKIQVTPPGFQLVFLPFADDKRKMPFTE
KIMATPEQVGKMKAIVEKLRFTYRSDSFENPVLQQHFRNLEALALDLMEP
EQAVDLTLPKVEAMNKRLGSLVDEFKELVYPPDYNPEGKVTKRKHDNEGS
GSKRPKVEYSEEELKTHISKGTLGKFTVPMLKEACRAYGLKSGLKKQELL
EALTKHFQD

(See also Reeves, W. H. and Sthoeger, Z. M., Molecular cloning of cDNA encoding the p70 (Ku) lupus autoantigen, J. Biol. Chem. 264 (9), 5047-5052 (1989).)

An amino acid sequence for a human lupus p80 (Ku) autoantigen protein is reported as follows (GenBank Accession No J04977; SEQ ID NO:89):

MVRSGNKAAVVLCMDVGFTMSNSIPGIESPFEQAKKVITMFVQRQVFAEN
KDEIALVLFGTDGTDNPLSGGDQYQNITVHRHLMLPDFDLLEDIESKIQP
GSQQADFLDALIVSMDVIQHETIGKKFEKRHIEIFTDLSSRFSKSQLDII
IHSLKKCDISLQFFLPFSLGKEDGSGDRGDGPFRLGGHGPSFPLKGITEQ
QKEGLEIVKMVMISLEGEDGLDEIYSFSESLRKLCVFKKIERHSIHWPCR
LTIGSNLSIRIAAYKSILQERVKKTWTVVDAKTLKKEDIQKETVYCLNDD
DETEVLKEDIIQGFRYGSDIVPFSKVDEEQMKYKSEGKCFSVLGFCKSSQ
VQRRFFMGNQVLKVFAARDDEAAAVALSSLIHALDDLDMVAIVRYAYDKR
ANPQVGVAFPHIKHNYECLVYVQLPFMEDLRQYMFSSLKNSKKYAPTEAQ
LNAVDALIDSMSLAKKDEKTDTLEDLFPTTKIPNPRFQRLFQCLLHRALH
PREPLPPIQQHIWNMLNPPAEVTTKSQIPLSKIKTLFPLIEAKKKDQVTA
QEIFQDNHEDGPTAKKLKTEQGGAHFSVSSLAEGSVTSVGSVNPAENFRV
LVKQKKASFEEASNQLINHIEQFLDTNETPYFMKSIDCIRAFREEAIKFS
EEQRFNNFLKALQEKVEIKQLNHFWEIVVQDGITLITKEEASGSSVTAEE
AKKFLAPKDKPSGDTAAVFEEGGDVDDLLDMI

(See also Yaneva, M., Wen, J., Ayala, A. and Cook, R., cDNA-derived amino acid sequence of the 86-kDa subunit of the Ku antigen, J. Biol. Chem. 264 (23), 13407-13411 (1989).)

An amino acid sequence for a human La protein/SS-B antigen is reported as follows (GenBank Accession No J04205 M11108; SEQ ID NO:90):

MAENGDNEKMAALEAKICHQIEYYFGDFNLPRDKFLKEQIKLDEGWVPLE
IMIKFNRLNRLTTDFNVIVEALSKSKAELMEISEDKTKIRRSPSKPLPEV
TDEYKNDVKNPSVYIKGFPTDATLDDIKEWLEDKGQVLNIQMRRTLHKAF
KGSIFVVFDSIESAKKFVETPGQKYKETDLLILFKDDYFAKKNEERKQNK
VEAKLRAKQEQEAKQKLEEDAEMKSLEEKIGCLLKFSGDLDDQTCREDLH
ILFSNHGEIKWIDFVRGAKEGIILFKEKAKEALGKAKDANNGNLQLRNKE
VTWEVLEGEVEKEALKKIIEDQQESLNKWKSKGRRFKGKGKGNKAAQPGS
GKGKVQFQGKKTKFASDDEHDEHDENGATGPVKRAREETDKEEPASKQQK
TENGAGDQ

(See also Chambers et al., Genomic structure and amino acid sequence domains of the human La autoantigen, J. Biol. Chem. 263 (34), 18043-18051 (1988).)

Bowel Autoantigens and Bystander Antigens

In an alternative embodiment of the present invention the autoantigen or bystander antigen may be a bowel autoantigen or bystander antigen for use to treat an autoimmune disease of the bowel.

The term “autoimmune disease of the bowel” as used herein includes any disease in which the bowel or a component of the bowel comes under autoimmune attack.

The main autoimmune diseases of the bowel are inflammatory bowel disease (IBD) and celiac (also known as coeliac) disease.

Inflammatory bowel disease (IBD) is the term generally applied to four diseases of the bowel, namely Crohn's disease, ulcerative colitis, indeterminate colitis, and infectious colitis.

Ulcerative colitis is a chronic inflammatory disease mainly affecting the large intestine. The course of the disease may be continuous or relapsing, mild or severe. The earliest lesion is typically an inflammatory infiltration with abscess formation at the base of the crypts of Lieberkuhn. Coalescence of these distended and ruptured crypts tends to separate the overlying mucosa from its blood supply, leading to ulceration. Signs and symptoms of the disease include cramping, lower abdominal pain, rectal bleeding, and frequent, loose discharges consisting mainly of blood, pus, and mucus with scanty fecal particles. A total colectomy may be required for acute severe or chronic, unremitting ulcerative colitis.

Crohn's disease (also known as regional enteritis or ulcerative ileitis) is also a chronic inflammatory disease of unknown etiology but, unlike ulcerative colitis, it can affect any part of the bowel. The most prominent feature of the disease is the granular, reddish-purple edematous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of the disease may be continuous or relapsing, mild or severe but, unlike ulcerative colitis, it is not curable by resection of the involved segment of bowel. Many patients with Crohn's disease require surgery at some point, but subsequent relapse is common and continuous medical treatment is usual.

Celiac disease (CD) is a disease of the intestinal mucosa and is usually identified in infants and children. Celiac disease is associated with an inflammation of the mucosa, which causes malabsorption. Individuals with celiac disease are intolerant to the protein gluten, which is present in foods such as wheat, rye and barley. When exposed to gluten, the immune system of an individual with celiac disease responds by attacking the lining of the small intestine.

The term “bowel autoantigen” as used herein includes any substance or a component thereof normally found within a mammal that, in an autoimmune disease of the bowel, becomes a target of attack by the immune system, preferably the primary (or a primary) target of attack. The term also includes antigenic substances that induce conditions having the characteristics of an autoimmune disease of the gut when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “bowel bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the bowel under autoimmune attack in an autoimmune disease of the bowel. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

It will be appreciated that combinations of bowel autoimmune/bystander antigens and bowel autoimmune/bystander antigenic determinants and/or polynucleotide sequences coding for them may also be used as appropriate.

Examples of bowel autoantigens and bystander antigens include, but are not limited to, gliadins and tissue transglutaminases (tTG) (associated with celiac disease; see Marsh, Nature Medicine 1997; 7:725-6) and tropomyosins, in particular tropomyosin isoform 5, (associated with ulcerative colitis).

Sjogren's Syndrome Autoantigens and Bystander Antigens

In an alternative embodiment of the present invention the autoantigen or bystander antigen may be a Sjogren's syndrome autoantigen or bystander antigen or antigenic determinant thereof, for use to treat an autoimmune disease of the bowel.

The term “Sjogren's syndrome autoantigen” as used herein includes any substance or a component thereof normally found within a mammal that, in Sjogren's syndrome, becomes a target of attack by the immune system, preferably the primary (or a primary) target of attack. The term also includes antigenic substances that induce conditions having the characteristics of Sjogren's syndrome when administered to mammals. Additionally, the term includes fragments comprising antigenic determinants (epitopes; preferably immunodominant epitopes) or epitope regions (preferably immunodominant epitope regions) of Sjogren's syndrome autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “Sjogren's syndrome bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in Sjogren's syndrome. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Some examples of Sjogren's syndrome autoantigens and Sjogren's syndrome bystander antigens include, but are not limited to, the following:

For example, an amino acid sequence for a human 52-kD SS-A/Ro autoantigen is reported as follows (GenBank Accession No M62800 M35041; SEQ ID NO:91):

MASAARLTMMWEEVTCPICLDPFVEPVSIECGHSFCQECISQVGKGGGSV
CAVCRQRFLLKNLRPNRQLANMVNNLKEISQEAREGTQGERCAVHGERLH
LFCEKDGKALCWVCAQSRKHRDHAMVPLEEAAQEYQEKLQVALGELRRKQ
ELAEKLEVEIAIKRADWKKTVETQKSRIHAEFVQQKNFLVEEEQRQLQEL
EKDEREQLRILGEKEAKLAQQSQALQELISELDRRCHSSALELLQEVIIV
LERSESWNLKDLDITSPELRSVCHVPGLKKMLRTCAVHITLDPDTANPWL
ILSEDRRQVRLGDTQQSIPGNEERFDSYPMVLGAQHFHSGKHYWEVDVTG
KEAWDLGVCRDSVRRKGHFLLSSKSGFWTIWLWNKQKYEAGTYPQTPLHL
QVPPCQVGIFLDYEAGMVSFYNITDHGSLIYSFSECAFTGPLRPFFSPGF
NDGGKNTAPLTLCPLNIGSQGSTDY

(See also Chan et al., Molecular definition and sequence motifs of the 52-kD component of human SS-A/Ro autoantigen, J. Clin. Invest. 87 (1), 68-76 (1991).)

An amino acid sequence for Human SS-A/Ro ribonucleoprotein autoantigen 60 kd subunit is reported as follows (GenBank Accession No M25077):

MEESVNQMQPLNEKQIANSQDGYVWQVTDMNRLHRFLCFGSEGGTYYIKE
QKLGLENAEALIRLIEDGRGCEVIQEIKSFSQEGRTTKQEPMLFALAICS
QCSDISTKQAAFKAVSEVCRIPTHLFTFIQFKKDLKESMKCGMWGRALRK
AIADWYNEKGGMALALAVTKYKQRNGWSHKDLLRLSHLKPSSEGLAIVTK
YITKGWKEVHELYKEKALSVETEKLLKYLEAVEKVKRTRDELEVIHLIEE
HRLVREHLLTNHLDSDEVWKALLQEMPLTALLRNLGKMTANSVLEPGNSE
VSLVCEKLCNEKLLKKARIHPFHILIALETYKTGHGLRGKLKWRPDEEIL
KALDAAFYKTFKTVEPTGKRFLLAVDVSASMNQRVLGSILNASTVAAAMC
MVVTRTEKDSYVVAFSDEMVPCPVTTDMTLQQVLMAMSQIPAGGTDCSLP
MIWAQKTNTPADVFIVFTDNETFAGGVHPAIALREYRKKMDIPAKLIVCG
MTSNGFTIADPDDRAIIQNTLLNKSF

(See also Ben-Chetrit et al., Isolation and characterization of a cDNA clone encoding the 60-kD component of the human SS-A/Ro ribonucleoprotein autoantigen, J. Clin. Invest. 83 (4), 1284-1292 (1989).)

Further sequences are provided, for example, under GenBank Accession Nos. NM_{—}003141.2 (Sjogren syndrome antigen A1 (52 kDa, ribonucleoprotein autoantigen SS-A/Ro) (SSA1)); NM_{—}004600.1 (Sjogren syndrome antigen A2 (60 kDa, ribonucleoprotein autoantigen SS-A/Ro) (SSA2)); NM_{—}003142.1, BC001289.1, BC020818.1 (Sjogren syndrome antigen B (autoantigen La) (SSB)); NM_{—}003731.1, BC000864.1 (Sjogren's syndrome nuclear autoantigen 1 (SSNA1)); NM_{—}006396.1, BC014791.1

(Sjogren's syndrome/scleroderma autoantigen 1 (SSSCA1)); AJ277541.1, AF282065.1 (SLA/LP autoantigen).

Thyroid Autoantigens and Bystander Antigens

In an alternative embodiment of the present invention the autoantigen or bystander antigen may be a thyroid autoantigen or bystander antigen or antigenic determinant thereof, for use to treat an autoimmune disease of the thyroid.

The term “thyroid autoimmune disease” as used herein includes any condition in which there is an autoimmune reaction to the thyroid or a component thereof. The best known autoimmune diseases of the thyroid include Graves' disease (also known as thyrotoxicosis), Hashimoto's thyroiditis and primary hypothyroidism. Further examples include atrophic autoimmune thyroiditis, primary myxoedema, asymptomatic thyroiditis, postpartal thyroiditis and neonatal hypothyroidism.

Diagnosis is typically based on the detection of autoantibodies in the patient. The three main thyroid autoantigens are the TSH receptor, thyroperoxidase (TPO, also known as microsomal antigen) and thyroglobulin (Tg) (Dawe, K., Hutchings, P., Champion, B., Cooke, A., Roitt, I., “Autoantigens in Thyroid diseases”, Springer Semin. Immunopathol. 14, 285-307, 1993).

The term “thyroid autoantigen” as used herein includes any substance or a component thereof normally found within a mammal that, in a thyroid autoimmune disease, becomes a target of attack by the immune system, preferably the primary (or a primary) target of attack. The term also includes antigenic substances that induce conditions having the characteristics of a thyroid autoimmune disease when administered to mammals. Additionally, the term includes fragments comprising antigenic determinants (epitopes; preferably immunodominant epitopes) or epitope regions (preferably immunodominant epitope regions) of autoantigens. In humans afflicted with an autoimmune disease, immunodominant epitopes or regions are fragments of antigens from (and preferably specific to) the tissue or organ (usually the thyroid gland) under autoimmune attack and recognized by a substantial percentage (e.g. a majority though not necessarily an absolute majority) of autoimmune attack T-cells.

The term “thyroid bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the thyroid gland under autoimmune attack. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Examples of thyroid autoantigens and thyroid bystander antigens include, but are not limited to, the thyroid stimulatory hormone (TSH) receptor (associated in particular with Grave's disease), thyroperoxidases (TPO; associated with Hashimoto's thyroiditis) and thyroglobulins (Tg).

For example, an amino acid sequence for a human thyroid stimulatory hormone receptor (TSHR) is reported as follows (GenBank Accession No M32215; SEQ ID NO:92):

MRPADLLQLVLLLDLPRDLGGMGCSSPPCECHQEEDFRVTCKDIQRIPSL
PPSTQTLKLIETHLRTIPSHAFSNLPNISRIYVSIDVTLQQLESHSFYNL
SKVTHIEIRNTRNLTYIDPDALKELPLLKFLGIFNTGLKMFPDLTKVYST
DIFFILEITDNPYMTSIPVNAFQGLCNETLTLKLYNNGFTSVQGYAFNGT
KLDAVYLNKNKYLTVIDKDAFGGVYSGPSLLDVSQTSVTALPSKGLEHLK
ELIARNTWTLKKLPLSLSFLHLTRADLSYPSHCCAFKNQKKIRGILESLM
CNESSMQSLRQRKSVNALNSPLHQEYEENLGDSIVGYKEKSKFQDTHNNA
HYYVFFEEQEDEIIGFGQELKNPQEETLQAFDSHYDYTICGDSEDMVCTP
KSDEFNPCEDIMGYKFLRIVVWFVSLLALLGNVFVLLILLTSHYKLNVPR
FLMCNLAFADFCMGMYLLLIASVDLYTHSEYYNHAIDWQTGPGCNTAGFF
TVFASELSVYTLTVITLERWYAITFAMRLDRKIRLRHACAIMVGGWVCCF
LLALLPLVGISSYAKVSICLPMDTETPLALAYIVFVLTLNIVAFVIVCCC
YVKIYITVRNPQYNPGDKDTKIAKRMAVLIFTDFICMAPISFYALSAILN
KPLITVSNSKILLVLFYPLNSCANPFLYAIFTKAFQRDVFILLSKFGICK
RQAQAYRGQRVPPKNSTDIQVQKVTHEMRQGLHNMEDVYELIEKSHLTPK
KQGQISEEYMQTVL

An amino acid sequence for a human thyroperoxidase (described as the primary autoantigen in human autoimmune thyroiditis (Hashimoto's thyroiditis) is reported as follows (GenBank Accession No M17755; SEQ ID NO:93):

MRALAVLSVTLVMACTEAFFPFISRGKELLWGKPEESRVSSVLEESKRLV
DTAMYATMQRNLKKRGILSGAQLLSFSKLPEPTSGVIARAAEIMETSIQA
MKRKVNLKTQQSQHPTDALSEDLLSIIANMSGCLPYMLPPKCPNTCLANK
YRPITGACNNRDHPRWGASNTALARWLPPVYEDGFSQPRGWNPGFLYNGF
PLPPVREVTRHVIQVSNEVVTDDDRYSDLLMAWGQYIDHDIAFTPQSTSK
AAFGGGSDCQMTCENQNPCFPIQLPEEARPAAGTACLPFYRSSAACGTGD
QGALFGNLSTANPRQQMNGLTSFLDASTVYGSSPALERQLRNWTSAEGLL
RVHGRLRDSGRAYLPFVPPRAPAACAPEPGNPGETRGPCFLAGDGRASEV
PSLTALHTLWLREHNRLAAALKALNAHWSADAVYQEARKVVGALHQIITL
RDYIPRILGPEAFQQYVGPYEGYDSTANPTVSNVFSTAAFRFGHATIHPL
VRRLDASFQEHPDLPGLWLHQAFFSPWTLLRGGGLDPLIRGLLARPAKLQ
VQDQLMNEELTERLFVLSNSSTLDLASINLQRGRDHGLPGYNEWREFCGL
PRLETPADLSTAIASRSVADKILDLYKHPDWIDVWLGGLABNFLPRARTG
PLFACLIGKQMKALRDGDWFWWENSHVFTDAQRRELEKHSLSRVICDNTG
LTRVPMDAFQVGKFPEDFESCDSITGMNLEAWRETFPQDDKCGFPESVEN
GDFVHCEESGRRVLVYSCRHGYELQGREQLTCTQEGWDFQPPLCKDVNEC
ADGAHPPCHASARCRNTKGGFQCLCADPYELGDDGRTCVDSGRLPRVTWI
SMSLAALLIGGFAGLTSTVICRWTRTGTKSTLPISETGGGTPELRCGKHQ
AVGTSPQRAAAQDSEQESAGMEGRDTHRLPRAL

Skin Autoantigens and Bystander Antigens

In an alternative embodiment of the present invention the autoantigen or bystander antigen may be a skin autoantigen or bystander antigen or antigenic determinant thereof, for use to treat an autoimmune disease of the skin.

The term “skin autoantigen” as used herein includes any substance or a component thereof normally found within a mammal that, in an autoimmune disease of the skin, such as Psoriasis or Vitiligo (or e.g. Pemphigus as mentioned above), becomes a target of attack by the immune system, preferably the primary (or a primary) target of attack. The term also includes antigenic substances that induce conditions having the characteristics of an autoimmune disease of the skin when administered to mammals. Additionally, the term includes fragments comprising antigenic determinants (epitopes; preferably immunodominant epitopes) or epitope regions (preferably immunodominant epitope regions) of skin autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “skin bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of the skin. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Endocrine Autoantigens and Bystander Antigens

In an alternative embodiment of the present invention the autoantigen or bystander antigen may be an enocrine autoantigen or bystander antigen or antigenic determinant thereof, for use to treat an autoimmune disease of an endocrine gland.

The term “endocrine autoantigen” as used herein includes any substance or a component thereof normally found within a mammal that, in an autoimmune disease of an endocrine gland, such as Autoimmune oophoritis (or e.g. Grave's disease or diabetes as mentioned above), becomes a target of attack by the immune system, preferably the primary (or a primary) target of attack. The term also includes antigenic substances that induce conditions having the characteristics of an autoimmune disease of the skin when administered to mammals. Additionally, the term includes fragments comprising antigenic determinants (epitopes; preferably immunodominant epitopes) or epitope regions (preferably immunodominant epitope regions) of endocrine autoantigens. In humans afflicted with an autoimmune disease, immunodominant epitopes or regions are fragments of antigens from (and preferably specific to) the tissue or organ under autoimmune attack and recognized by a substantial percentage (e.g. a majority though not necessarily an absolute majority) of autoimmune attack T-cells.

The term “endocrine bystander antigen” as used herein includes any substance capable of eliciting an immune response, including proteins, protein fragments, polypeptides, peptides, glycoproteins, nucleic acids, polysaccharides or any other immunogenic substance that is, or is derived from, a component of the organ or tissue under autoimmune attack in an autoimmune disease of an endocrine gland. The term includes but is not limited to autoantigens and fragments thereof such as antigenic determinants (epitopes) involved in autoimmune attack. In addition, the term includes antigens normally not exposed to the immune system which become exposed in the locus of autoimmune attack as a result of autoimmune tissue destruction.

Modulators of Notch Signalling

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists. Preferably the modulator is an agonist of Notch signalling, and preferably an agonist of the Notch receptor (e.g. an agonist of the Notch1, Notch2, Notch3 and/or Notch4 receptor).

The active agent of the present invention may be an organic compound or other chemical. In one embodiment, a modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the modulator will be an amino acid sequence or a chemical derivative thereof, or a combination thereof. In another preferred embodiment, the modulator will be a nucleotide sequence—which may be a sense sequence or an anti-sense sequence. The modulator may also be an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:

(i) Fab, the fragment which contains a monovalent antigen-binding fragment of an antibody molecule can be produced by digestion of whole antibody with the enzyme papain to yield an intact light chain and a portion of one heavy chain;

(ii) Fab′, the fragment of an antibody molecule can be obtained by treating whole antibody with pepsin, followed by reduction, to yield an intact light chain and a portion of the heavy chain; two Fab′ fragments are obtained per antibody molecule;

(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;

(iv) scFv, including a genetically engineered fragment containing the variable region of a heavy and a light chain as a fused single chain molecule.

General methods of making these fragments are known in the art. (See, for example, Harlow and Lane, Antibodies: A Laboratory Manual, Cold Spring Harbor Laboratory, New York (1988), which is hereby incorporated herein by reference).

Modulators may be synthetic compounds or natural isolated compounds.

In one form the modulator of the Notch signalling pathway may be a protein for Notch signalling transduction. By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein is a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In a preferred form of the invention the signalling may be specific signalling, meaning that the signal results substantially or at least predominantly from the Notch signalling pathway, and preferably from Notch/Notch ligand interaction, rather than any other significant interfering or competing cause, such as cytokine signalling. Thus, in a preferred embodiment, Notch signalling excludes cytokine signalling. The Notch signalling pathway is described in more detail below.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or by employing other mechanisms to transduce extracellular signals.

According to one aspect of the present invention the active agent may be Notch or a fragment thereof which retains the signalling transduction ability of Notch or an analogue of Notch which has the signalling transduction ability of Notch.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a protein which is for Notch signalling activation we mean a molecule which is capable of activating Notch, the Notch signalling pathway or any one or more of the components of the Notch signalling pathway.

Suitably, the modulator of Notch signalling may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term as used herein therefore includes naturally occurring protein ligands such as Delta and Serrate/Jagged and their biologically active fragments as well as antibodies to the Notch receptor, peptidomimetics and small molecules which have corresponding biological effects to the natural ligands. Preferably the Notch ligand interacts with the Notch receptor by binding.

Particular examples of naturally occurring mammalian Notch ligands identified to date include the Delta family, for example Delta or Delta-like 1 (Genbank Accession No. AF003522—Homo sapiens), Delta-3 (Genbank Accession No. AF084576—Rattus norvegicus) and Delta-like 3 (Mus musculus) (Genbank Accession No. NM_{—}016941—Homo sapiens) and U.S. Pat. No. 6,121,045 (Millennium), Delta-4 (Genbank Accession Nos. AB043894 and AF 253468—Homo sapiens) and the Serrate family, for example Serrate-1 and Serrate-2 (WO97/01571, WO96/27610 and WO92/19734), Jagged-1 (Genbank Accession No. U73936—Homo sapiens) and Jagged-2 (Genbank Accession No. AF029778—Homo sapiens), and LAG-2. Homology between family members is extensive.

In one embodiment, an activator of Notch signalling may be a constitutively active Notch receptor or Notch intracellular domain, or a polynucleotide encoding such a receptor or intracellular domain.

In an alternative embodiment, an activator of Notch signalling may act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include the polypeptides involved in the Ras/MAPK cascade catalysed by Deltex, polypeptides involved in the proteolytic cleavage of Notch such as Presenilin and polypeptides involved in the transcriptional regulation of Notch target genes, suitably in a constitutively active form.

By polypeptide for Notch signalling activation is also meant any polypeptides expressed as a result of Notch activation and any polypeptides involved in the expression of such polypeptides, or polynucleotides coding for such polypeptides.

In another embodiment a modulator of Notch signalling may be a molecule which is capable of enhancing Notch-Notch ligand interactions. A molecule may be considered to enhance Notch-Notch ligand interactions if it is capable of enhancing the interaction of Notch with its ligands, preferably to an extent sufficient to provide therapeutic efficacy.

Preferably when the inhibitor is a receptor or a nucleic acid sequence encoding a receptor, the receptor is activated. Thus, for example, when the agent is a nucleic acid sequence, the receptor is preferably constitutively active when expressed.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to components of the Notch signalling pathway. These include the use of phage display systems for expressing signalling proteins, and using a culture of transfected E. coli or other microorganism to produce the proteins for binding studies of potential binding compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is, for example, about 10 to 40 amino acids long, preferably 10 to 35 amino acids.

The amino acid sequence may be prepared and isolated from a suitable source, or it may be made synthetically or it may be prepared by use of recombinant DNA techniques.

Within the definitions of “proteins”, “polypeptides” and “peptides” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

A protein used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp, Asn | Aspartic, | |
| | | Asparagine | GAT, GAC, AAT, AAC |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | |
| | | Glutamine | GAA, GAG, CAA, CAG |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, suitably between about 8 and about 1500 amino acids in length, for example between about 8 and about 745 amino acids in length, preferably about 8 to about 300, more preferably about 8 to about 200 amino acids, for example about 10 to about 50 or 100 amino acids in length. “Peptide” refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino-acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Nucleotide Sequences

Where the modulator of Notch signalling or antigen/antigenic determinant comprises a nucleotide sequence it may suitably be codon optimised for expression in mammalian cells. In a preferred embodiment, such sequences are optimised in their entirety.

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and also derivatised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for an active protein, peptide or polypeptide.

As indicated above, with respect to sequence homology, similarity or identity, preferably there is at least 75%, more preferably at least 85%, more preferably at least 90% homology, similarity or identity to the reference sequences, preferably over the entire length of the reference sequences. More preferably there is at least 95%, more preferably at least 98%, homology, similarity or identityover the entire length of the reference sequences.

Nucleotide homology comparisons may be conducted as described below. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (University of Wisconsin, U.S.A.; Devereux). Examples of other software than can perform sequence comparisons include, but are not limited to, the BLAST package, FASTA (Atschul et al. (1990) J. Mol. Biol. 403-410 (Atschul)) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

The five BLAST programs, available at the website maintained by the U.S. National Institutes of Health National Center for Biotechnology Information, perform the following tasks:

blastp—compares an amino acid query sequence against a protein sequence database.

blastn—compares a nucleotide query sequence against a nucleotide sequence database.

blastx—compares the six-frame conceptual translation products of a nucleotide query sequence (both strands) against a protein sequence database.

tblastn—compares a protein query sequence against a nucleotide sequence database dynamically translated in all six reading frames (both strands).

tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see databases maintained by the U.S. National Institutes of Health National Center for Biotechnology Information). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided by the U.S. National Institutes of Health National Center for Biotechnology Information.

In some aspects of the present invention, no gap penalties are used when determining sequence identity.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Polynucleotide Hybridisation

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein includes “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC (1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency preferably refers to conditions that permit hybridisation of only those nucleic acid sequences that form stable hybrids in 1 M Na+at 65-68° C. High stringency conditions can be provided, for example, by hybridisation in an aqueous solution containing 6×SSC, 5× Denhardt's, 1% SDS (sodium dodecyl sulphate), 0.1 Na+pyrophosphate and 0.1 mg/ml denatured salmon sperm DNA as non specific competitor. Following hybridisation, high stringency washing may be done in several steps, with a final wash (about 30 min) at the hybridisation temperature in 0.2−0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Nucleotide sequences can be obtained in a number of ways. Variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the target protein or protein for T cell signalling modulation encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Transfection and Expression

For recombinant production, host cells can be genetically engineered to incorporate expression systems or polynucleotides of the invention. Introduction of a polynucleotide into the host cell can be effected by methods described in many standard laboratory manuals, such as Davis et al. and Sambrook et al., such as calcium phosphate transfection, DEAE-dextran mediated transfection, transvection, microinjection, cationic lipid-mediated transfection, electroporation, transduction, scrape loading, ballistic introduction and infection. In will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc.) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

Chemical Coupling

Chemically coupled sequences can be prepared from individual proteins sequences and coupled using known chemically coupling techniques. The conjugate can be assembled using conventional solution- or solid-phase peptide synthesis methods, affording a fully protected precursor with only the terminal amino group in deprotected reactive form. This function can then be reacted directly with a protein or polypeptide or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein or polypeptide or a derivative thereof may be attached through e.g. amide, ester, or disulphide bond formation. Cross-linking reagents which can be utilized are discussed, for example, in Neans, G. E. and Feeney, R. E., Chemical Modification of Proteins, Holden-Day, 1974, pp. 39-43.

Polypeptides and Polynucleotides for Notch Signalling Transduction

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299—Homo sapiens).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as disulphide-linked heterodimeric molecules consisting of an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta may require cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struh1). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

S3 processing occurs only in response to binding of Notch ligands Delta or Serrate/Jagged. The post-translational modification of the nascent Notch receptor in the Golgi (Munro; Ju) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin; Hicks). Although Drosophila has a single Fringe gene, vertebrates are known to express multiple genes (Radical, Manic and Lunatic Fringes) (Irvine).

Signal transduction from the Notch receptor can occur via two different pathways. The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

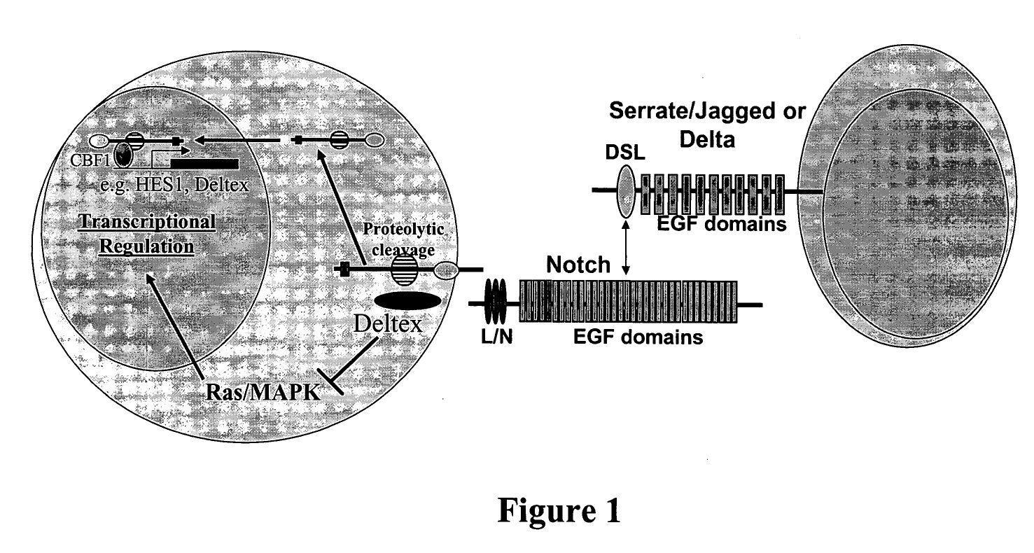

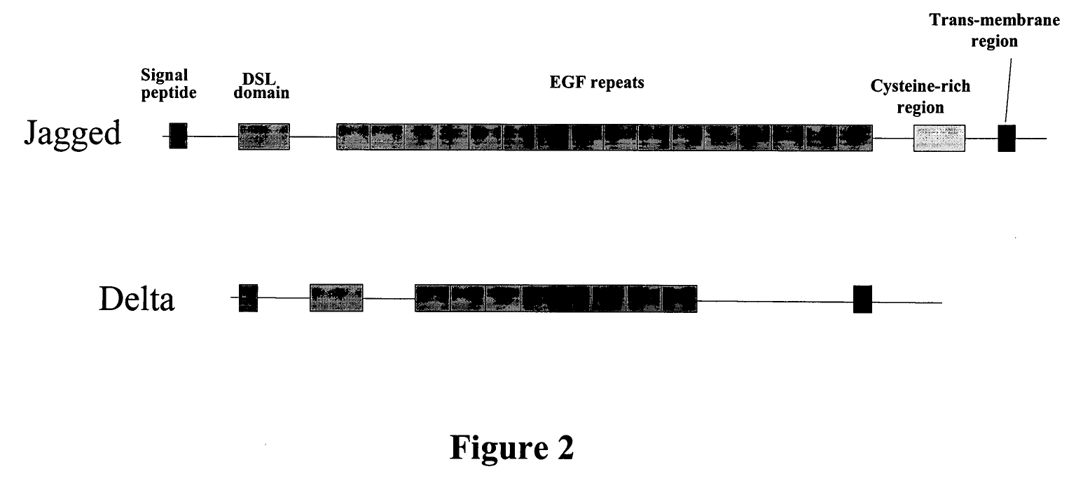

Thus, signal transduction from the Notch receptor can occur via two different pathways both of which are illustrated in FIG. 1. Target genes of the Notch signalling pathway include Deltex, genes of the Hes family (Hes-1 in particular), Enhancer of Split [E(spl)] complex genes, IL-10, CD-23, CD-4 and D11-1.

Deltex, an intracellular docking protein, replaces Su(H) as it leaves its site of interaction with the intracellular tail of Notch. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

IL-10 (interleukin-10) is a factor produced by Th2 helper T-cells. It is a co-regulator of mast cell growth and shows extensive homology with the Epstein-Barr bcrfi gene. Although it is not known to be a direct downstream target of the Notch signalling pathway, its expression has been found to be strongly upregulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated coincident with Notch activation. The sequence for CD-23 may be found in GenBank ref. No. GI1783344.

Dlx-1 (distalless-1) (McGuiness) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

Examples of mammalian Notch ligands identified to date include the Delta family, for example Delta-1 (Genbank Accession No. AF003522—Homo sapiens), Delta-3 (Genbank Accession No. AF084576—Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Homology between family members is extensive.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example Ausubel et al., Current Protocols in Molecular Biology (1995), John Wiley & Sons, Inc. and databases maintained by the U.S. National Institutes of Health National Center for Biotechnology Information).

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain and up to 14 or more EGF-like repeats on the extracellular surface.

In addition, suitable homologues will be capable of binding to a Notch receptor. Binding may be assessed by a variety of techniques known in the art including in vitro binding assays.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence: (SEQ ID NO:94)

Cys Xaa Xaa Xaa Xaa Xaa Xaa Xaa Xaa Cys Xaa Xaa
Xaa Cys Xaa Xaa Xaa Xaa Xaa Xaa Xaa Xaa Xaa Xaa
Xaa Cys Xaa Xaa Xaa Xaa Xaa Xaa Xaa Cys Xaa Xaa
Xaa Xaa Xaa Xaa Xaa Xaa Cys

Preferably the DSL domain may include most or all of the following consensus amino acid sequence (SEQ ID NO:95):

Cys Xaa Xaa Xaa ARO ARO Xaa Xaa Xaa Cys Xaa Xaa
Xaa Cys BAS NOP BAS ACM ACM Xaa ARO NOP ARO Xaa
Xaa Cys Xaa Xaa Xaa NOP Xaa Xaa Xaa Cys Xaa Xaa
NOP ARO Xaa NOP Xaa Xaa Cys

wherein:

ARO is an aromatic amino acid residue, such as tyrosine, phenylalanine, tryptophan or histidine;

NOP is a non-polar amino acid residue such as glycine, alanine, proline, leucine, isoleucine or valine;

BAS is a basic amino acid residue such as arginine or lysine; and

ACM is an acid or amide amino acid residue such as aspartic acid, glutamic acid, asparagine or glutamine.

Preferably the DSL domain may include most or all of the following consensus amino acid sequence (SEQ ID NO:96):

Cys Xaa Xaa Xaa Tyr Tyr Xaa Xaa Xaa Cys Xaa Xaa
Xaa Cys Arg Pro Arg Asx Asp Xaa Phe Gly His Xaa
Xaa Cys Xaa Xaa Xaa Gly Xaa Xaa Xaa Cys Xaa Xaa
Gly Trp Xaa Gly Xaa Xaa Cys

(wherein Xaa may be any amino acid and Asx is either aspartic acid or asparagine).

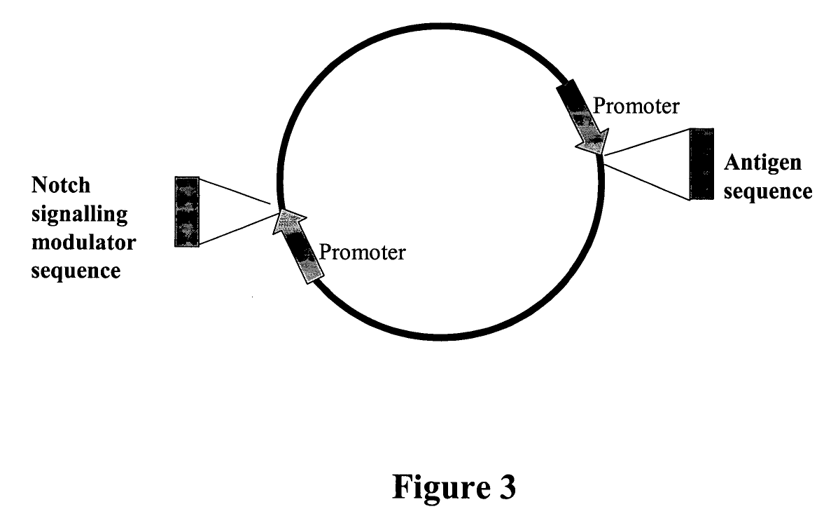

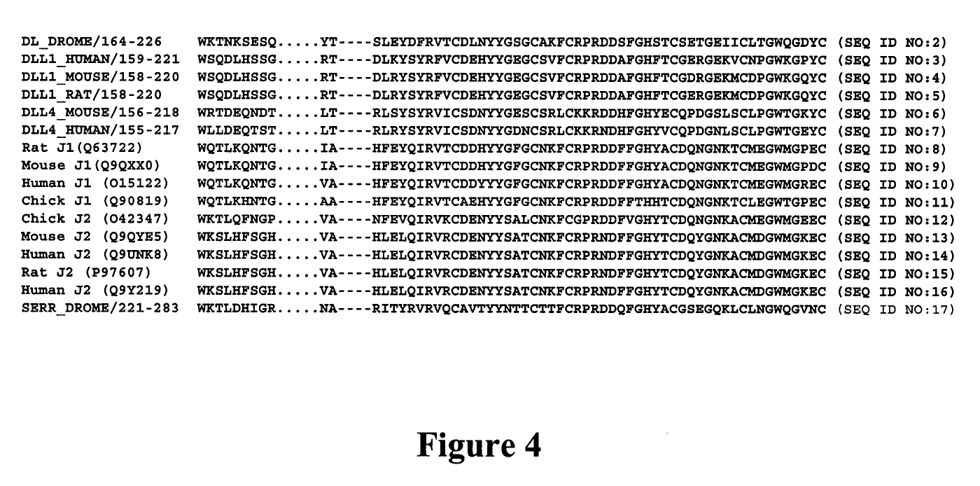

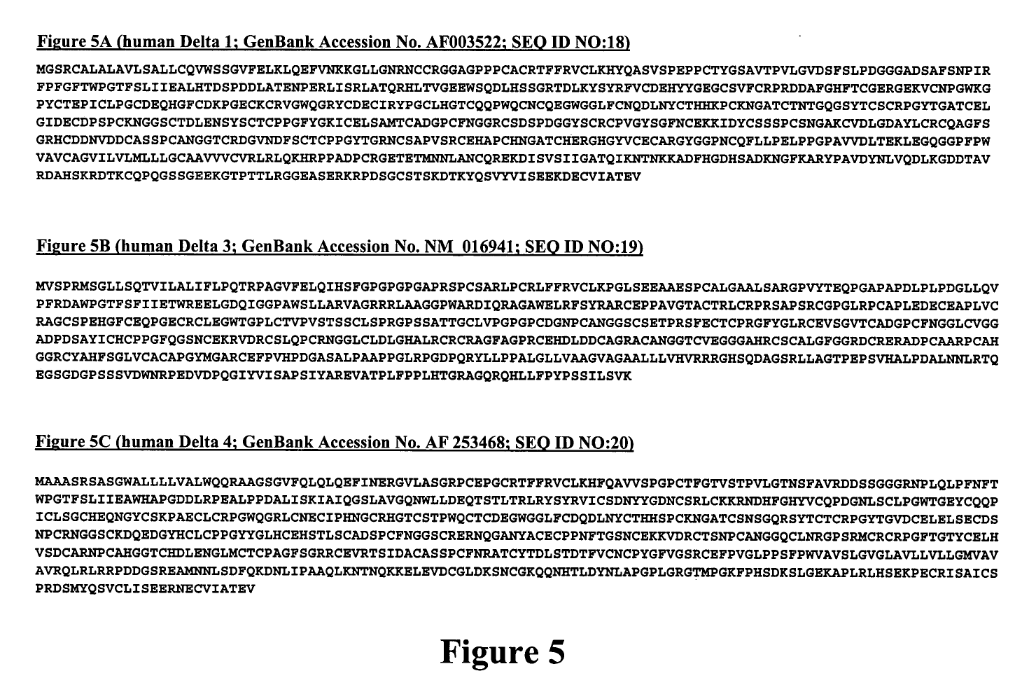

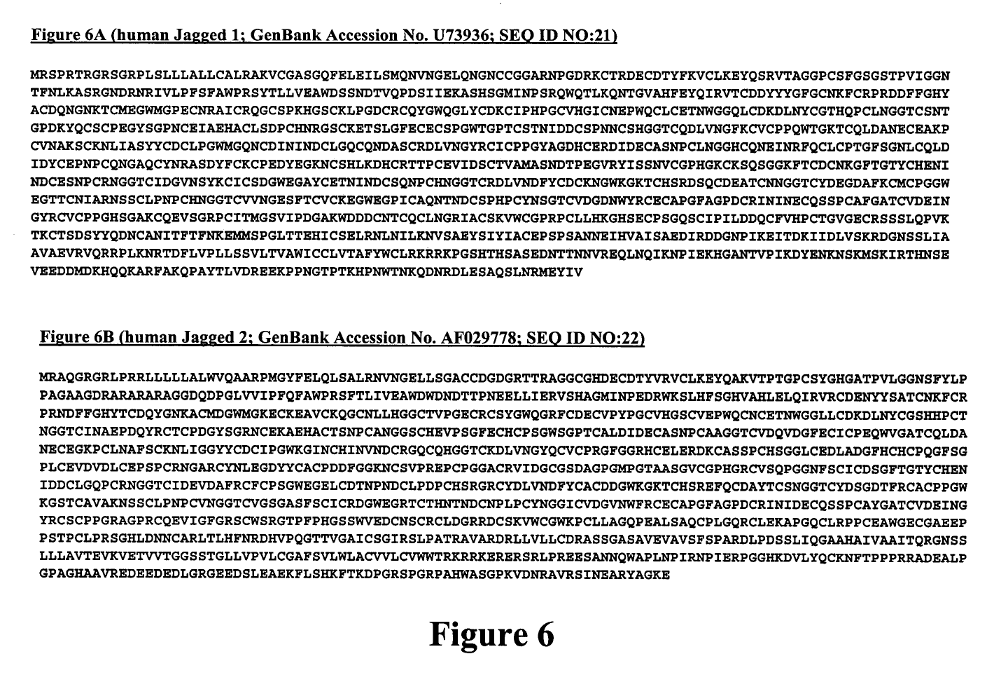

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

It will be appreciated that the term “DSL domain” as used herein includes sequence variants, fragments, derivatives and mimetics having activity corresponding to naturally occurring domains.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2 .

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain (SEQ ID NO:97):

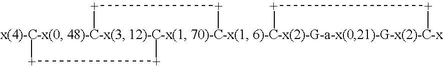

wherein:

‘C’: conserved cysteine involved in a disulfide bond.

‘G’: often conserved glycine

‘a’: often conserved aromatic amino acid

‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

It will be appreciated that the term “EGF domain” as used herein includes sequence variants, fragments, derivatives and mimetics having activity corresponding to naturally occurring domains.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X % identical to another sequence can be determined conventionally using known computer programs. For example, the best overall match between a query sequence and a subject sequence, also referred to as a global sequence alignment, can be determined using a program such as the FASTDB computer program based on the algorithm of Brutlag et al. (Comp. App. Biosci. (1990) 6:237-245). In a sequence alignment the query and subject sequences are either both nucleotide sequences or both amino acid sequences. The result of the global sequence alignment is given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

Suitably, for example, a Notch ligand N-terminal domain for use in the present invention may have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 2.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 3.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 4.

The term “heterologous amino acid sequence” or “heterologous nucleotide sequence” as used herein means a sequence which is not found in the native sequence (e.g. in the case of a Notch ligand sequence is not found in the native Notch ligand sequence) or its coding sequence. Typically, for example, such a sequence may be an IgFc domain or a tag such as a V5His tag.

Monitoring of Notch Signalling: Screens and Assays

Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of D11-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al. (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al. (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

Conjugates

As rioted above, the invention further provides a conjugate comprising first and second sequences, wherein the first sequence comprises an autoimmune antigen or autoimmune antigenic determinant or a polynucleotide sequence coding for such an autoimmune antigen or autoimmune antigenic determinant and the second sequence comprises a polypeptide or polynucleotide for Notch signalling modulation. The conjugates of the present invention may be protein/polypeptide or polynucleotide conjugates.

Where the conjugate is a polynucleotide conjugate, it may suitably take the form of a polynucleotide vector such as a plasmid comprising a polynucleotide sequence coding for an autoimmune antigen or autoimmune antigenic determinant and a polynucleotide sequence coding for a modulator of the Notch signalling pathway, wherein preferably each sequence is operably linked to regulatory elements necessary for expression in eukaryotic cells. A schematic representation of one such form of vector is shown in FIG. 3.

The term “operably linked” means that the components described are in a relationship permitting them to function in their intended manner. A regulatory sequence “operably linked” to a coding sequence is peferably ligated in such a way that expression of the coding sequence is achieved under condition compatible with the regulatory/control sequences.

Suitably the polynucleotide sequence coding for the modulator of the Notch signalling pathway may be a nucleotide sequence coding for a Notch ligand such as Delta1, Delta3, Delta4, Jagged1 or Jagged 2, or a biologically active fragment, derivative or homologue of such a sequence. Where intended for human therapy, suitably sequences based on human sequences may be used.

Preferably the polynucleotide sequence coding for the modulator of the Notch signalling pathway may be a nucleotide sequence coding for a Notch ligand DSL domain and at least 1 to 20, suitably at least 2 to 15, suitably at least 2 to 10, for example at least 3 to 8 EGF-like domains. Suitably the DSL and EGF-like domain sequences are or correspond to mammalian sequences. In one embodiment the polynucleotide sequence coding for the modulator of the Notch signalling pathway may further comprise a transmembrane domain (so that the sequence may be expressed on a cell surface, as a membrane protein or polypeptide) and, suitably, a Notch ligand intracellular domain. Preferred sequences include human sequences such as human Delta1, Delta3, Delta4, Jagged1 or Jagged2 sequences.

If desired, the polynucleotide sequence that encodes the autoantigen or bystander antigen may further include a nucleotide sequence that encodes a signal sequence which directs trafficking of the antigen or antigenic determinant within a cell to which it is administered. For example, such a signal sequence may direct the antigen or antigenic determinant to be secreted or to be localized to the cytoplasm, the cell membrane, the endoplasmic reticulum, or a lysosome.

Regulatory elements for DNA expression include a promoter and a polyadenylation signal. In addition, other elements, such as a Kozak region, may also be included if desired. Initiation and termination signals are regulatory elements which are often considered part of the coding sequence.

Examples of suitable promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein. Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Suitably an epithelial cell promoter such as SPC may be used.

Examples of suitable polyadenylation signals include but are not limited to SV40 polyadenylation signals and LTR polyadenylation signals. For example, the SV40 polyadenylation signal used in plasmid pCEP4 (Invitrogen, San Diego Calif.), referred to as the SV40 polyadenylation signal, may be used.

In addition to the regulatory elements required for DNA expression, other elements may also be included in the conjugate. Such additional elements include enhancers which may, for example, be selected from human Actin, human Myosin, human Hemoglobin, human muscle creatine and viral enhancers such as those from CMV, RSV and EBV.

When adminstered to and taken up by a cell, the nucleotide conjugate may for example remain present in the cell as a functioning extrachromosomal molecule and/or integrate into the cell's chromosomal DNA. DNA may be introduced into cells where it remains as separate genetic material in the form of a plasmid or plasmids. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be administered to the cell. It is also possible, for example, to provide the conjugate in the form of a minichromosome including a centromere, telomeres and an origin of replication.

If desired, conjugates may be provided with mammalian origin of replication in order to maintain the construct extrachromosomally and produce multiple copies of the construct in the cell. For example, plasmids pCEP4 and pREP4 from Invitrogen (San Diego, Calif.) contain the Epstein Barr virus origin of replication and nuclear antigen EBNA-1 coding region which produces high copy episomal replication without integration.

In order to maximize protein production, regulatory sequences may be selected which are well suited for gene expression in the type of cells the construct is to be administered to. Moreover, codons may be selected which are most efficiently transcribed in the cell.

Intracellular trafficking signals may also be included as appropriate. Such signals are well known in the art, and include the following:

In some embodiments, the expressed antigen or antigenic determinant may be directed to be secreted by inclusion of an N-terminal hydrophobic sequence. When RNA is translated, the hydrophobic sequence at the N terminal causes the protein to bind to the rough endoplasmic reticulum (RER). The hydrophobic sequence is subsequently clipped off by a protease and the protein is secreted. Thus, if desired, the antigen or antigenic determinant may include an N terminal hydrophobic leader sequence which will direct secretion of the antigen or antigenic determinant when expressed in a cell.

Alternatively, the expressed antigen or antigenic determinant may be directed to be membrane bound by inclusion of an N-terminal hydrophobic sequence and an internal hydrophobic region. As in the secreted forms, when RNA is translated, the hydrophobic sequences causes the protein to bind to the RER. The N terminal hydrophobic sequence is subsequently clipped off by a protease. The protein follows the same secretion pathway but the internal hydrophobic sequence prevents secretion and the protein becomes membrane bound. Thus, if desired, the expressed antigen or antigenic determinant may include an N terminal hydrophobic leader sequence and an internal hydrophobic sequence which will result in the antigen or antigenic determinant, or part therof, becoming membrane bound when expressed in a cell.

In some alternative embodiments, the expressed antigen or antigenic determinant may be directed to be localized in the cytosol by omitting an N-terminal hydrophobic sequence. When RNA is translated, the protein does not bind to the rough endoplasmic reticulum and the protein becomes cytosolic. Thus, if desired, the expressed antigen or antigenic determinant is free of an N terminal hydrophobic leader sequence so that it becomes cytosolic when expressed in a cell.

In some alternative embodiments, the expressed antigen or antigenic determinant is localized in the lysosome by inclusion of a sequence (such as DKQTLL; SEQ ID NO:106) which directs localization to lysosomes. Thus, if desired, the antigen or antigenic determinant may include a sequence (such as DKQTLL) so that it is directed to the lysosome when expressed in the cell.

In some embodiments, expressed antigens or antigenic determinants are directed to be localized from the Golgi body back to the ER by including a sequence (such as KDEL; SEQ ID NO:107) at the C terminal which directs localization to the ER. One example of such an “ER recycling signal” is reported to be the C terminal sequence of the E19 protein from adenovirus. That protein is localized to the ER where it binds to the MHCs and effectively keeps them from loading proteins which are presented by the MHC at the surface where they complex with T cell receptors as part of immune response induction. The E109 protein is a hexapeptide DEKKMP (SEQ ID NO:108).

Depending upon the type of immune response sought to be modulated, different intracellular localization may be desirable. In the case of Class I immune responses, proteins synthesized within a cell are degraded and transported into the ER where they are loaded onto MHCs which then move to the cell surface and complex with T cell receptors of CD8⁺ T cells. This action encourages CTL responses. In the case of Class II immune responses, proteins are complexed with antigen presenting cells (APCs) which interact with CD4⁺ T cells, engaging helper T cells including those associated with antibody responses.

In order to enhance Class I immune responses, localization of proteins to the cytosol or ER allows for such proteins to be more accessible to the Class I pathway.

In order to enhance Class II immune responses, localization of proteins to the transmembrane or lysosomes, or secretion of the protein allows such proteins to be more accessible to the Class II pathway.

Further examples of localization leaders are provided, for example, in Biocca, S. et al. 1990 EMBO J. 9:101-108.

In some embodiments, nucleotide conjugates may code for lysosomal targeting doublets at the C terminal tail of the expressed antigen or antigenic determinant. By including the doublets LL and/or YQ and/or QY the expressed antigen or antigenic determinant is directed to a lysosome.

Facilitating Agents

In some embodiments, polynucleotides may be delivered in conjunction with administration of a facilitating agent. Facilitating agents which are administered in conjunction with nucleic acid molecules may be administered as a mixture with the nucleic acid molecule or administered separately simultaneously, before or after administration of nucleic acid molecules. Examples of facilitators include benzoic acid esters, anilides, amidines, urethans and the hydrochloride salts thereof such as those of the family of local anesthetics.

Examples of esters include: benzoic acid esters such as piperocaine, meprylcaine and isobucaine; para-aminobenzoic acid esters such as procaine, tetracaine, butethamine, propoxycaine and chloroprocaine; meta-aminobenzoic acid esters including metabuthamine and primacaine; and para-ethoxybenzoic acid esters such as parethoxycaine. Examples of anilides include lidocaine, etidocaine, mepivacaine, bupivacaine, pyrrocaine and prilocaine. Other examples of such compounds include dibucaine, benzocaine, dyclonine, pramoxine, proparacaine, butacaine, benoxinate, carbocaine, methyl bupivacaine, butasin picrate, phenacaine, diothan, luccaine, intracaine, nupercaine, metabutoxycaine, piridocaine, biphenamine and the botanically-derived bicyclics such as cocaine, cinnamoylcocaine, truxilline and cocaethylene and all such compounds complexed with hydrochloride.

The facilitating agent may be administered prior to, simultaneously with or subsequent to the genetic construct. The facilitating agent and the genetic construct may be formulated in the same composition.

Bupivacaine-HCl is chemically designated as 2-piperidinecarboxamide, 1-butyl-N-(2,6-dimethylphenyl)-monohydrochloride, monohydrate and is widely available commercially for pharmaceutical uses from many sources including from Astra Pharmaceutical Products Inc. (Westboro, Mass.) and Sanofi Winthrop Pharmaceuticals (New York, N.Y.), Eastman Kodak (Rochester, N.Y.). Bupivacaine is commercially formulated with and without methylparaben and with or without epinephrine. Any such formulation may be used. It is commercially available for pharmaceutical use in concentration of 0.25%, 0.5% and 0.75% which may be used on the invention. Alternative concentrations, particularly those between 0.05%-1.0% which elicit desirable effects may be prepared if desired. Suitably, for example, about 250 μg to about 10 mg of bupivacaine may be administered.

Particles and Particle Delivery

In one embodiment, modulators of Notch signalling may be administered on delivery particles, preferably microparticles, preferably in combination with antigens or antigenic determinants or, preferably, nucleic acids coding for antigens or antigenic determinants, to modulate immune responses to such antigens or antigenic determinants.

Thus, for example, in one embodiment the present invention provides a delivery particle suitable for administration to a subject to modulate an immune response to an antigen or antigenic determinant which comprises (e.g. is coated or impregnated with):
i) a modulator of Notch signalling (such as a nucleic acid coding for a Notch receptor agonist, such as a Notch ligand or active fragment, variant or derivative); and
ii) an antigen or antigenic determinant or, preferably, a nucleic acid coding for an antigen or antigenic determinant.

In one embodiment such a particle may comprise (e.g. be coated or impregnated with):
i) a modulator of Notch signalling (such as a nucleic acid coding for a Notch receptor agonist, such as a Notch ligand or active fragment, variant or derivative); and
ii) an autoantigen, bystander antigen, allergen, pathogen antigen or graft antigen or an antigenic determinant thereof or, preferably, a nucleic acid coding for such an antigen or antigenic determinant;

Such a particle may be administered to reduce an immune response to said antigen or antigenic determinant.

A variety of particles and delivery systems may be used in the present invention, including but not limited to, the following:

(i) Biolistic Particle Delivery

In one embodiment, particles according to the present invention may be administered by a needleless or “ballistic” (biolistic) delivery mechanism. A range of such delivery systems are known in the art. One system, developed by Powderject Vaccines, is particularly useful and a variety of suitable forms and embodiments are described, for example, in the following publications, which are incorporated herein by reference:

WO03011380 Silencing Device And Method For Needleless Syringe; WO03011379 Particle Cassette, Method And Kit Therefor; WO02101412 Spray Freeze-Dried Compositions; WO02100380 Production Of Hard, Dense Particles; WO02055139 Needleless Syringe; WO0243774 Nucleic Acid Immunization; WO0219989 Alginate Particle Formulation; WO0207803 Needleless Syringe; WO0193829 Powder Compositions; WO0183528 Nucleic Acid Immunization; WO0168167 Apparatus And Method For Adjusting The Characteristics Of A Needleless Syringe; WO0134185 Induction Of Mucosal Immunity By Vaccination Via The Skin Route; WO0133176 Apparatus And Method For Dispensing Small Quantities Of Particles; WO0105455 Needleless Syringe; WO0063385 Nucleic Acid Immunization; WO0062846 Needleless Syringe; WO0054827 Needleless Syringe; WO0053160 Delivery Of Microparticle Formulations Using Needleless Syringe Device For Sustained-Release Of Bioactive Compounds; WO0044421 Particle Delivery Device; WO0026385 Nucleic Acid Constructs For Genetic Immunization; WO0023592 Minimal Promoters And Uses Thereof; WO0019982 Spray Coated Microparticles For Use In Needleless Syringes; WO9927961 Transdermal Delivery Of Particulate Vaccine Compositions; WO9908689 Mucosal Immunization Using Particle-Mediated Delivery Techniques; WO9901169 Syringe And Capsule Therefor; WO9901168 Drug Particle Delivery; WO9821364 Method And Apparatus For Preparing Sample Cartridges For A Particle Acceleration Device; WO9813470 Gas-Driven Particle Delivery Device; WO9810750 Nucleic Acid Particle Delivery; WO9748485 Method For Providing Dense Particle Compositions For Use In Transdermal Particle Delivery; WO9734652 Needleless Syringe With Therapeutic Agent Particles Entrained In Supersonic Gas Flow.

As described, for example, in 20020165176 A1, particle-mediated methods for delivering such nucleic acid preparations are known in the art. Thus, once prepared and suitably purified, the nucleic acid molecules can be coated onto carrier particles (e.g., core carriers) using a variety of techniques known in the art. Carrier particles are selected from materials which have a suitable density in the range of particle sizes typically used for intracellular delivery from a particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

Suitable particles include metal particles such as, tungsten, gold, platinum and iridium carrier particles. Tungsten and gold particles are preferred. Tungsten particles are readily available in average sizes of 0.5 to 2.0 um in diameter. Gold particles or microcrystalline gold (e.g., gold powder A1570, available from Engelhard Corp., East Newark, N.J) may also be used. Gold particles provide uniformity in size (available from Alpha Chemicals in particle sizes of 1-3 um, or available from Degussa, South Plainfield, N.J. in a range of particle sizes including 0.95 um) and low toxicity. Microcrystalline gold provides a diverse particle size distribution, typically in the range of 0.1-5 um. The irregular surface area of microcrystalline gold provides for highly efficient coating with nucleic acids.

A large number of methods are known and have been described for coating or precipitating polynucleotides such as DNA or RNA onto articles such as gold or tungsten particles. Typically such methods combine a predetermined amount of gold or tungsten with plasmid DNA, CaCl₂ and spermidine. The resulting solution is suitably vortexed continually during the coating procedure to ensure uniformity of the reaction mixture. After precipitation of the nucleic acid, the coated particles can for example be transferred to suitable membranes and allowed to dry prior to use, coated onto surfaces of a sample module or cassette, or loaded into a delivery cassette for use in particular particle-mediated delivery instruments.

Following their formation, carrier particles coated with the nucleic acid preparations can be delivered to a subject using particle-mediated delivery techniques.

Various particle acceleration devices suitable for particle-mediated delivery are known in the art, and are all suited for use in the practice of the invention. Current device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

If desired, these particle acceleration devices can be provided in a preloaded condition containing a suitable dosage of the coated carrier particles comprising the polynucleotide vaccine composition, with or without additional influenza vaccine compositions and/or a selected adjuvant component. The loaded syringe can be packaged in a hermetically sealed container.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 1000 ug, more preferably 0.01 to 10.0 ug of nucleic acid molecule per dose, depends on the subject to be treated. The exact amount necessary will vary depending on the age and general condition of the individual being immunized and the particular nucleotide sequence or peptide selected, as well as other factors. An appropriate effective amount can be readily determined by one of skill in the art.

The formulated compositions may suitably be prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in International Publication No. WO 97/48485, incorporated herein by reference.

These methods can be used to obtain nucleic acid particles having a size ranging from about 0.01 to about 250 um, preferably about 10 to about 150 um, and most preferably about 20 to about 60 um; and a particle density ranging from about 0.1 to about 25 g/cm³, and a bulk density of about 0.5 to about 3.0 g/cm³, or greater.

Single unit dosages or multidose containers, in which the particles may be packaged prior to use, may suitably comprise a hermetically sealed container enclosing a suitable amount of the particles. The particulate compositions can be packaged as a sterile formulation, and the hermetically sealed container can thus be designed to preserve sterility of the formulation until use in the methods of the invention. If desired, the containers can be adapted for direct use in a needleless syringe system. Such containers can take the form of capsules, foil pouches, sachets, cassettes, and the like. Appropriate needleless syringes are described herein above.

The container in which the particles are packaged can further be labeled to identify the composition and provide relevant dosage information. In addition, the container can be labeled with a notice in the form prescribed by a governmental agency, for example the Food and Drug Administration, wherein the notice indicates approval by the agency under Federal law of the manufacture, use or sale of the composition contained therein for human administration.

Following their formation, the particulate composition (e.g., powder) can be delivered transdermally to the subject's tissue using a suitable transdermal delivery technique. Various particle acceleration devices suitable for transdermal delivery of the substance of interest are known in the art, and will find use in the practice of the invention. A particularly preferred transdermal delivery system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect™ needleless syringe device”). Other needleless syringe configurations are known in the art and are described herein.

Suitably, the particulate compositions will be delivered via a powder injection method, e.g., delivered from a needleless syringe system such as those described in commonly owned International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 um, preferably ranging from about 1-70 um. Particles larger than about 250 urn can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 1-70 um can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labeled as described above.

Compositions containing a therapeutically effective amount of the powdered molecules described herein can be delivered to any suitable target tissue via the above-described needleless syringes. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 ug/kg to 100 ug/kg of nucleic acid molecule per dose, depends on the subject to be treated. Doses for other pharmaceuticals, such as physiological active peptides and proteins, generally range from about 0.1 ug to about 20 mg, preferably 10 ug to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

(ii) Liposome Particle Delivery

In an alternative embodiment, particles may take the form of lipid complexes and/or liposomes.

For example, lipid-nucleic acid formulations can be formed by combining the nucleic acid with a preformed cationic liposome (see, U.S. Pat. Nos. 4,897,355, 5,264,618, 5,279,833 and 5,283,185). In such methods, the nucleic acid is attracted to the cationic surface charge of the liposome and the resulting complexes are thought to be of the liposome-covered “sandwich-type.”

Liposome-based delivery of polynucleotides is also described, for example, in N. J. Caplen, et al., Liposome-mediated CFTR gene transfer to the nasal epithelium of patients with cystic fibrosis, Nature Medicine, 1(1995) 39; M. Cotten and E. Wagner, Non-viral approaches to gene therapy, Current opinion in biotechnology, (1993) 705-710; A. Singhal and L. Huang, Gene transfer in mammalian cells using liposomes as carriers, in Gene Therapeutics: Methods and Applications of Direct Gene Transfer, J. A. Wolff, Editor. 1994, Birkhauser: Boston; and J. P. Schonfield and C. T. Caskey, Non-viral approaches to gene therapy, Brit. Med. J., 51(1995) 56.

(iii) Delivery of Particles for Uptake by Cells

In an alternative embodiment, particles may be administered for active uptake by cells, for example by phagocytosis, as described for example in U.S. Pat. No. 5,783,567 (Pangaea), which is herein incorporated by reference.

As described, for example, in U.S. Pat. No. 5,783,567, phagocytosis of microparticles by macrophages and other antigen presenting cells (APCs) is an effective means for introducing the nucleic acid into these cells. Phagocytosis by these cells can be increased by maintaining a particle size preferably below about 20 um, and preferably below about 11 um. The type of polymer used in the microparticle can also affect the efficiency of uptake by phagocytic cells, as discussed below.

The microparticles can be delivered directly into the bloodstream (i.e., by intravenous or intraarterial injection or infusion) if uptake by the phagocytic cells of the reticuloendothelial system (RES) is desired. Alternatively, one can target, via subcutaneous injection, take-up by the phagocytic cells of the draining lymph nodes. The microparticles can also be introduced intradermally (i.e., to the APCs of the skin, such as dendritic cells and Langerhans cells). Another useful route of delivery (particularly for DNAs encoding tolerance-inducing polypeptides) is via the gastrointestinal tract, e.g., orally. Alternatively, the microparticles can be introduced into organs such as the lung (e.g., by inhalation of powdered microparticles or of a nebulized or aerosolized solution containing the microparticles), where the particles are picked up by the alveolar macrophages, or may be administered intranasally or buccally.

Once a phagocytic cell phagocytoses the microparticle, the nucleic acid is released into the interior of the cell. Upon release, it can perform its intended function: for example, expression by normal cellular transcription/translation machinery.

Because these microparticles are passively targeted to dendritic cells, macrophages and other types of phagocytic cells, they represent a means for modulating immune function. Macrophages serve as professional APCs, expressing both MHC class I and class II molecules.

Suitable polymeric material may be obtained from commercial sources or can be prepared by known methods. For example, polymers of lactic and glycolic acid can be generated as described in U.S. Pat. No. 4,293,539 or purchased from Aldrich.

Alternatively, or in addition, the polymeric matrix can include, for example, polylactide, polyglycolide, poly(lactide-co-glycolide), polyanhydride, polyorthoester, polycaprolactone, polyphosphazene, proteinaceous polymer, polypeptide, polyester, or polyorthoester.

Polymeric particles containing nucleic acids are suitably prepared using a double emulsion technique, for example, as follows: First, the polymer is dissolved in an organic solvent. A preferred polymer is polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing or shaking, and in a preferred method, the mixture can be sonicated. Most preferable is any method by which the nucleic acid receives the least amount of damage in the form of nicking, shearing, or degradation, while still allowing the formation of an appropriate emulsion. For example, acceptable results can be obtained with a Vibra-cell model VC-250 sonicator with a ⅛″ microtip probe, at setting #3.

During this process, the polymer forms into minute “microparticles,” each of which contains some of the nucleic acid-containing solution. If desired, one can isolate a small amount of the nucleic acid at this point in order to assess integrity, e.g., by gel electrophoresis.

The first emulsion is then added to an organic solution. The solution can be comprised of, for example, methylene chloride, ethyl acetate, or acetone, preferably containing polyvinyl alcohol (PVA), and most preferably having a 1:100 ratio of the weight of PVA to the volume of the solution. The first emulsion is generally added to the organic solution with stirring in a homogenizer or sonicator. For example, one can use a Silverson Model L4RT homogenizer (⅝″ probe) set at 7000 RPM for about 12 seconds. A 60 second homogenization time would be too harsh at this homogenization speed.

This process forms a second emulsion which is subsequently added to another organic solution with stirring (e.g., in a homogenizer). In a preferred method, the latter solution is 0.05% w/v PVA. The resultant microparticles are washed several times with water to remove the organic compounds. Particles can be passed through sizing screens to selectively remove those larger than the desired size. If the size of the microparticles is not crucial, one can dispense with the sizing step. After washing, the particles can either be used immediately or be lyophilized for storage.

The size distribution of the microparticles prepared by the above method can be determined with a COULTERM™ counter. This instrument provides a size distribution profile and statistical analysis of the particles. Alternatively, the average size of the particles can be determined by visualization under a microscope fitted with a sizing slide or eyepiece.

If desired, the nucleic acid can be extracted from the microparticles for analysis by the following procedure. Microparticles are dissolved in an organic solvent such as chloroform or methylene chloride in the presence of an aqueous solution. The polymer stays in the organic phase, while the DNA goes to the aqueous phase. The interface between the phases can be made more distinct by centrifugation. Isolation of the aqueous phase allows recovery of the nucleic acid. To test for degradation, the extracted nucleic acid can be analyzed by HPLC or gel electrophoresis.

To increase the recovery of nucleic acid, additional organic solvents, such as phenol and chloroform, can be added to the dissolved microparticles, prior to the addition of the aqueous solution. Following addition of the aqueous solution, the nucleic acid enters the aqueous phase, which can easily be partitioned from the organic phase after mixing. For a clean interface between the organic and aqueous phases, the samples should be centrifuged. The nucleic acid is retrieved from the aqueous phase by precipitation with salt and ethanol in accordance with standard methods.

Microparticles containing nucleic acid can be injected into mammals intramuscularly, intravenously, intraarterially, intradermally, intraperitoneally, or subcutaneously, or they can be introduced into the gastrointestinal tract or the respiratory tract, e.g., by inhalation of a solution or powder containing the microparticles. Expression of the nucleic acid may be monitored by an appropriate method.

Vectors for Introduction and Expression of Polynucleotides in Cells

An important aspect of the present invention is the use of delivery agents to introduce selected polynucleotide sequences into cells in vitro and, preferably, in vivo, followed by expression of the selected gene in the host cell. Thus, the nucleic acids in the particles are typically in the form of vectors that are capable of being expressed in the desired subject host cell. Promoter, enhancer, stress or chemically-regulated promoters, antibiotic-sensitive or nutrient-sensitive regions, as well as therapeutic protein encoding sequences, may be included as required.

As described, for example, in U.S. Pat. No. 5,976,567 (Inex), the expression of natural or synthetic nucleic acids is typically achieved by operably linking a nucleic acid of interest to a promoter (which may be either constitutive or inducible), preferably incorporating the construct into an expression vector, and introducing the vector into a suitable host cell. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors may be suitable for replication and integration in prokaryotes, eukaryotes, or preferably both. See, Giliman and Smith (1979), Gene, 8: 81-97; Roberts et al. (1987), Nature, 328: 731-734; Berger and Kimmel, Guide to Molecular Cloning Techniques, Methods in Enzymology, volume 152, Academic Press, Inc., San Diego, Cailf. (Berger); Sambrook et al. (1989), MOLECULAR CLONING—A LABORATORY MANUAL (2nd ed.) Vol. 1-3, Cold Spring Harbor Laboratory, Cold Spring Harbor Press, N.Y., (Sambrook); and F. M. Ausubel et al., CURRENT PROTOCOLS IN MOLECULAR BIOLOGY, eds., Current Protocols, a joint venture between Greene Publishing Associates, Inc. and John Wiley & Sons, Inc., (1994 Supplement) (Ausubel). Product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources.

Vectors to which foreign nucleic acids are operably linked may be used to introduce these nucleic acids into host cells and mediate their replication and/or expression. “Cloning vectors” are useful for replicating and amplifying the foreign nucleic acids and obtaining clones of specific foreign nucleic acid-containing vectors. “Expression vectors” mediate the expression of the foreign nucleic acid. Some vectors are both cloning and expression vectors.

An expression vector typically comprises a eukaryotic transcription unit or “expression cassette” that contains all the elements required for the expression of exogenous genes in eukaryotic cells. A typical expression cassette contains a promoter operably linked to the DNA sequence encoding a desired protein and signals required for efficient polyadenylation of the transcript.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated. Suitable promoters include the immediate early promoter from human cytomegalovirus (hCMV) and its associated intron A sequence (see e.g. WO0023592 for a suitable minimal promoter).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Another suitable enhancer element is the HBV 3′-enhancer and HBV preS2 5′-UTR (see for example GenBank Accession No AF462041). Other enhancer/promoter combinations that are suitable for the present invention include those drived from polyoma virus, human or murine cytomegalovirus, the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In addition to a promoter sequence, the expression cassette should also contain a transcription termination region downstream of the structural gene to provide for efficient termination. The termination region may be obtained from the same source as the promoter sequence or may be obtained from a different source.

If the mRNA encoded by the selected structural gene is to be efficiently translated, polyadenylation sequences are also commonly added to the vector construct (e.g. Rabbit B-globin pA: GenBank Accession No V00882). Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40, or a partial genomic copy of a gene already resident on the expression vector.

In addition to the elements already described, the expression vector of the present invention may typically contain other specialized elements intended to increase the level of expression of cloned nucleic acids or to facilitate the identification of cells that carry the transduced DNA. For instance, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The expression vectors of the present invention will typically contain both prokaryotic sequences that facilitate the cloning of the vector in bacteria as well as one or more eukaryotic transcription units that are expressed only in eukaryotic cells, such as mammalian cells. The prokaryotic sequences are preferably chosen such that they do not interfere with the replication of the DNA in eukaryotic cells.

Selected genes are normally be expressed when the DNA sequence is functionally inserted into a vector. “Functionally inserted” means that it is inserted in proper reading frame and orientation and operably linked to proper regrulatory elements. Typically, a gene will be inserted downstream from a promoter and will be followed by a stop codon, although production as a hybrid protein followed by cleavage may be used, if desired.

Expression vectors containing regulatory elements from eukaryotic viruses such as retroviruses are typically used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A.sup.+, pMTO10/A.sup.+, pMAMneo-5, baculovirus pDSVE, and any other vector allowing expression of proteins under the direction of the SV-40 early promoter, SV-40 later promoter, metallothionein promoter, murine mammary turnor virus promoter, Rous sarcoma virus promoter, polyhedrin promoter, or other promoters shown effective for expression in eukaryotic cells.

While a variety of vectors may be used, it should be noted that viral vectors such as retroviral vectors are useful for modifying eukaryotic cells because of the high efficiency with which the retroviral vectors transfect target cells and integrate into the target cell genome. Additionally, the retroviruses harboring the retroviral vector are capable of infecting cells from a wide variety of tissues.

In addition to the retroviral vectors mentioned above, cells may be lipofected with adeno-associated viral vectors. See, e.g., Methods in Enzymology, Vol. 185, Academic Press, Inc., San Diego, Calif. (D. V. Goeddel, ed.) (1990) or M. Krieger (1990), Gene Transfer and Expression—A Laboratory Manual, Stockton Press, New York, N.Y., and the references cited therein. Adeno associated viruses (AAVs) require helper viruses such as adenovirus or herpes virus to achieve productive infection. In the absence of helper virus functions, AAV integrates (site-specifically) into a host cell's genome, but the integrated AAV genome has no pathogenic effect. The integration step allows the AAV genome to remain genetically intact until the host is exposed to the appropriate environmental conditions (e.g., a lytic helper virus), whereupon it re-enters the lytic life-cycle. Samulski (1993), Current Opinion in Genetic and Development, 3: 74-80, and the references cited therein provides an overview of the AAV life cycle. See also West et al. (1987), Virology, 160: 38-47; Carter et al. (1989), U.S. Pat. No. 4,797,368; Carter et al. (1993), WO 93/24641; Kotin (1994), Human Gene Therapy, 5: 793-801; Muzyczka (1994), J. Clin. Invest., 94: 1351 and Samulski, supra, for an overview of AAV vectors.

Plasmids designed for producing recombinant vaccinia, such as pGS62, (Langford, C. L. et al. (1986), Mol. Cell. Biol., 6: 3191-3199) may also be used. This plasmid consists of a cloning site for insertion of foreign nucleic acids, the P7.5 promoter of vaccinia to direct synthesis of the inserted nucleic acid, and the vaccinia TK gene flanking both ends of the foreign nucleic acid.

For convenience, vectors may typically further comprise selectable markers which result in nucleic acid amplification such as the sodium, potassium ATPase, thymidine kinase, aminoglycoside phosphotransferase, hygromycin B phosphotransferase, xanthine-guanine phosphoribosyl transferase, CAD (carbamyl phosphate synthetase, aspartate transcarbamylase, and dihydroorotase), adenosine deaminase, dihydro folate reductase, and asparagine synthetase and ouabain selection. Alternatively, high yield expression systems not involving nucleic acid amplification are also suitable, such as using a bacculovirus vector in insect cells, with the encoding sequence under the direction of the polyhedrin promoter or other strong baculovirus promoters.

Treatable Conditions

Preferably the modulation of immune response is effected by control of immune cell, preferably T-cell, preferably peripheral T-cell, activity.

Suitably the modulation of immune response comprises reducing an immune response to an autoantigen or bystander antigen.

Suitably the modulation of immune response comprises promoting immune tolerance to an autoantigen or bystander antigen.

In one embodiment, the modulation of immune response comprises reducing the activity of effector T-cells, for example helper (T_{H}) or cytotoxic (T_{C}) T-cells. Preferably, the reduction of activity is with respect to effector T-cells specific for an autoantigen or bystander antigen. Preferably, the activity of effector T-cells specific for an autoantigen or bystander antigen is reduced more than the activity of effector T-cells of other specificities.

Alternatively or in addition, the modulation of immune response comprises increasing the activity of regulatory (also called suppressor) T-cells, for example Tr1 or Th3 T-cells. Preferably, the increase of activity is with respect to regulatory T-cells specific for an autoantigen or bystander antigen. Preferably, the activity of regulatory T-cells specific for an autoantigen or bystander antigen is increased more than the activity of regulatory T-cells of other specificities.

Examples of autoimmune disorders range from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions, Goodpasture's disease and pemphigus.

In more detail, organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrome, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis,

Administration

Suitably the active agents are administered in combination with a pharmaceutically acceptable carrier or diluent. The pharmaceutically acceptable carrier or diluent may be, for example, sterile isotonic saline solutions, or other isotonic solutions such as phosphate-buffered saline. The conjugates of the present invention may be admixed with any suitable binder(s), lubricant(s), suspending agent(s), coating agent(s), solubilising agent(s).

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient.

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilisers and preservatives as may be required.

For some applications, active agents may be administered orally in the form of tablets containing excipients such as starch or lactose, or in capsules or ovules either alone or in admixture with excipients, or in the form of elixirs, solutions or suspensions containing flavouring or colouring agents.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes. Active agents may be adminstered by conventional DNA delivery techniques, such as DNA vaccination etc., or injected or otherwise delivered with needleless systems, such as ballistic delivery on particles coated with the DNA for delivery to the epidermis or other sites such as mucosal surfaces.

In general, a therapeutically effective oral or intravenous dose is likely to range from 0.01 to 50 mg/kg body weight of the subject to be treated, preferably 0.1 to 20 mg/kg. The conjugate may also be administered by intravenous infusion, at a dose which is likely to range from 0.001-10 mg/kg/hr.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Tablets or capsules of the conjugates may be administered singly or two or more at a time, as appropriate. It is also possible to administer the conjugates in sustained release formulations.

Active agents may also be injected parenterally, for example intracavernosally, intravenously, intradermally, intramuscularly or subcutaneously

For parenteral administration, active agents may suitably be used in the form of a sterile aqueous solution which may contain other substances, for example enough salts or monosaccharides to make the solution isotonic with blood.

For buccal or sublingual administration, agents may be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Imnmunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

The present invention also provides pharmaceutical kits useful, for example, in the treatment or prevention of autoimmune allergy, which comprise one or more containers containing a pharmaceutical composition comprising a therapeutically effective amount of a modulator of Notch signalling and one or more containers containing a pharmaceutical composition comprising an autoimmune antigen or autoimmune antigenic determinant or a polynucleotide coding for an autoantigen or bystander antigen. Such kits may further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, may also be included if required.

The agents of the present invention can be administered by any suitable means including, but not limited to, for example, oral, rectal, nasal, topical (including transdermal, aerosol, buccal and sublingual), vaginal and parenteral (including subcutaneous, intramuscular, intravenous and intradermal) routes of administration. The modulator of Notch signalling and the autoantigen or bystander antigen may be administered by the same or separate routes. For example, the modulator of Notch signalling may be administered systemically whilst the autoantigen or bystander antigen may be administered locally, or both agents may be adminstered systemically or both agents may be adminstered locally.

Alternatively or in addition, one, both or more agents may be administered directly to an organ or tissue which is subject to autoimmune disease, e.g. an arthritic joint in the case of rheumatoid arthritis or the thyroid gland in the case of thyroiditis.

It will be appreciated that it may be appropriate to administer more than one dose of either the modulator of Notch signalling and/or the autoantigen or bystander antigen.

By “simultaneously” is meant that the modulator of the Notch signalling pathway and the autoantigen or bystander antigen or biologically active derivative, homologue or variant therof are administered at substantially the same time, and suitably together in the same formulation.

By “contemporaneously” it is meant that the modulator of the Notch signalling pathway and the autoantigen or bystander antigen, coding polynucleotide or biologically active derivative, homologue or variant therof are administered closely in time, e.g., the autoantigen or bystander antigen, coding polynucleotide or biologically active derivative, homologue or variant therof is administered within from about one minute to within about one day before or after the modulator of the Notch signalling pathway is administered. Any contemporaneous time is useful. However, it will often be the case that when not administered simultaneously, the modulator of the Notch signalling pathway and the autoantigen or bystander antigen, coding polynucleotide or biologically active derivative, homologue or variant therof will be administered within about one minute to within about eight hours, and preferably within less than about one to about four hours. When administered contemporaneously, the modulator of the Notch signalling pathway and the autoantigen or bystander antigen, coding polynucleotide or biologically active derivative, homologue or variant therof are preferably administered at the same site on the patient/subject. The term “same site” includes the exact location, but can be within about 0.5 to about 15 centimetres, preferably from within about 0.5 to about 5 centimetres.

The term “separately” as used herein means that the modulator of the Notch signalling pathway and the autoantigen or bystander antigen, coding polynucleotide or biologically active derivative, homologue or variant thereof are administered at an interval, for example at an interval of about a day to several weeks or months. The active agents may be administered in either order.

Likewise, the modulator of the Notch signalling pathway may be administered more frequently than the autoantigen or bystander antigen, coding polynucleotide or biologically active derivative, homologue or variant therof or vice versa.

The term “sequentially” as used herein means that the modulator of the Notch signalling pathway and the autoantigen or bystander antigen , coding polynucleotide or biologically active derivative, homologue or variant therof are administered in sequence, for example at an interval or intervals of minutes, hours, days or weeks. If appropriate the active agents may be administered in a regular repeating cycle.

It may also be appropriate to administer an autoantigen or bystander antigen directly to an organ or tissue which is subject to autoimmune disease. For example, in rheumatoid arthritis a bystander antigen (e.g. Type II collagen) which has been administered in simultaneous, contemporaneous, separate or sequential combination with a modulator of Notch signalling may be further administered to the affected arthritic joint to provide initial activation T-cells, especially regulatory T-cells.

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin RS, et al. (1998) Gene Therapy 5: 718-722 (Coffin)). Culture conditions may include other cytokines such as GM-CSF or IL-4 for the maintenance and, or activity of the dendritic cells or other antigen presenting cells.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Introduction of Nucleic Acid Sequences into APCs and T-Cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences encoding the enhancers of Notch ligand expression and/or activity will be operably linked to control sequences, including promoters/enhancers and other expression regulation signals.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

The resulting T-cells and/or APCs that comprise nucleic acid constructs capable of up-regulating Notch ligand expression are now ready for use. If required, a small aliquot of cells may be tested for up-regulation of Notch ligand expression as described above. The cells may be prepared for administration to a patient or incubated with T-cells in vitro (ex vivo).

Tolerisation Assays

Any of the assays described above (see “Assays”) can be adapted to monitor or to detect reduced reactivity and tolerisation in immune cells for use in clinical applications. Such assays will involve, for example, detecting increased Notch-ligand expression or activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-0-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more agents capable of activating the Notch signalling pathway are then typically added to the culture medium together with the antigen of interest. The antigen or antigenic determinant may be added before, after or at substantially the same time as the agent(s). Cells are typically incubated with the agent(s) and antigen for at least one hour, preferably at least 3 hours, at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142. APCs transfected with a nucleic acid construct directing the expression of, for example Serrate, may be used as a control.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of a Notch signalling are now ready for use.

Preparation of Regulatory T Cells (and B Cells) Ex Vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different agent capable of activating Notch signalling to that used with the APC, then the T cell will not be brought into contact with the different agent used to upregulate Notch signalling in the APC. Alternatively, the T cell may be incubated with the agent first to activate Notch signalling, washed, resuspended and then incubated with the primed APC in the absence of both the agent(s) used to upregulate APC Notch signaling and the agent(s) used to upregulate Notch signalling in the T cell. Once primed APCs have been prepared, it is not always necessary to administer any agent(s) to the T cell since the primed APC may itself be capable of promoting immunotolerance leading to increased Notch signalling T cell, for example via Notch/Notch ligand interactions between the primed APC and T cell.

Incubations will typically be for at least 1 hour, preferably at least 3, 6, 12, 24, 36 or more hours, in suitable culture medium at 37° C. T cells transfected with a nucleic acid construct directing the expression of, for example Delta, may be used as a control. Induction of immunotolerance may be determined, for example, by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Alternatively, the T cell may be incubated with a first agent to activate Notch signalling, washed, resuspended and then incubated with a primed APC in the absence of both the agent(s) used to treat the APC and the agent(s) used to treat the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3, 6, 12, 24, 36 or more hours, in suitable culture medium at 37° C. Induction of immunotolerance may be determined by subsequently challenging T cells with antigen and measuring IL-2 production compared with control cells not exposed to APCs.

T cells or B cells which have been primed in this way may be used according to the invention to promote or increase immunotolerance in other T cells or B cells.

Various preferred features and embodiments of the present invention will now be described in more detail by way of non-limiting examples.

### EXAMPLE 1

### hDelta1-IgG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, e.g. Genbank Accession No AF003522) into the expression vector pCONγ (Lonza Biologics, Slough, UK) and expressing the resulting construct in CHO cells (see WO 03/041735, Example 1). The amino acid sequence (SEQ ID NO:98) of the resulting expressed fusion protein was as follows:

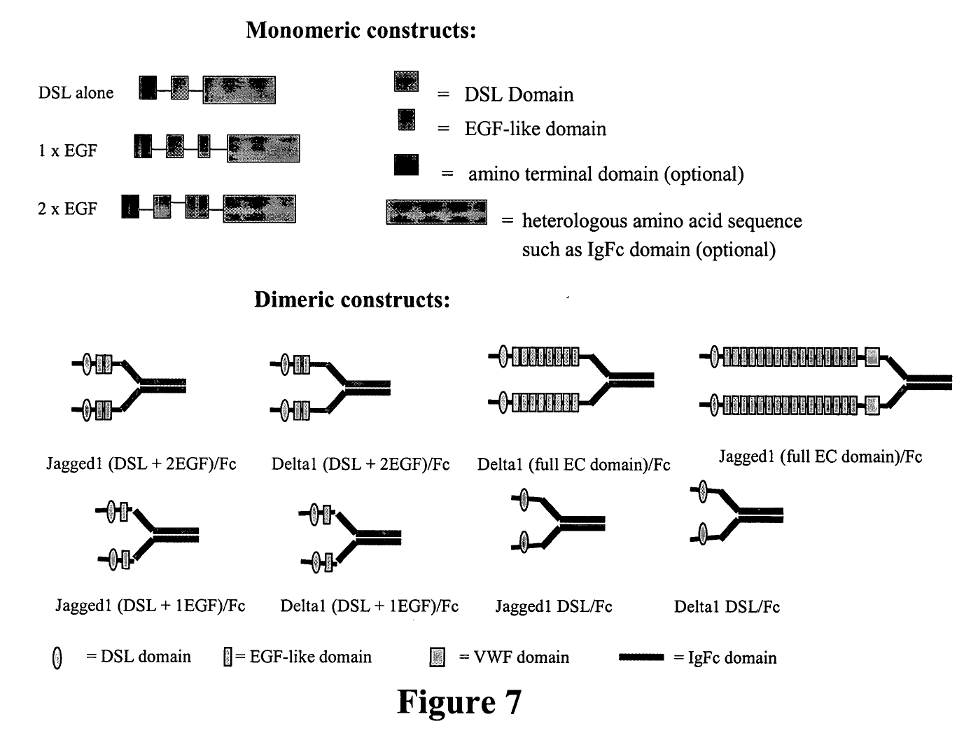

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERSISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECRYPGCLHGTCQQPWQCNCQEGWGGLFCNQDLNYCTHHKPCKNG
ATCTNTGQGSYTCSCRPGYTGATCELGIDECDPSPCKNGGSCTDLENSYS
CTCPPGFYGKICELSAMTCADGPCFNGGRCSDSPDGGYSCRCPVGYSGFN
CEKKIDYCSSSPCSNGAKCVDLGDAYLCRCQAGFSGRIICDDNVDDCASS
PCANGGTCRDGVNDFSCTCPPGYTGRNCSAPVSRCEHAPCHNGATCHERG
HGYVCECARGYGGPNCQFLLPELPPGPAVVDLTEKLEASTKGPSVFPLAP
CSRSTSESTAALGCLVKDYFPEPVTVSWNSGALTSGVHTFPAVLQSSGLY
SLSSVVTVPSSSLGTKTYTCNVDHKPSNTKVDKRVESKYGPPCPSCPAPE
FLGGPSVFLFPPKPKDTLMISRTPEVTCVVVDVSOEDPEVQFNWYVDGVE
VHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEALH
NHYTQKSLSLSLGK

Wherein the first underlined sequence is the signal peptide (cleaved from the mature protein) and the second underlined sequence is the IgG4 Fc sequence. The protein normally exists as a dimer linked by disulphide bonds (see e.g. schematic representation in FIG. 7).

### EXAMPLE 2

### Dynabeads ELISA Assay Method For Detecting Notch Signalling Activity

(i) CD4+ Cell Purification

Spleens were removed from mice (variously Balb/c females, 8-10 weeks, C57B/6 females, 8-10 weeks, D011.10 transgenic females, 8-10 weeks) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHC0₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-IgG4Fc fusion protein (10 μg/ml; as described above). The plates were incubated for 2-3 hours at 37° C. then washed again with DPBS before cells (prepared as described above) were added.

(iii) Primary Polyclonal Stimulation and ELISA

CD4+ cells were cultured in 96 well, flat-bottomed plates pre-coated according to (ii) above. Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of R10F medium was then added to each well to give a final volume of 200 μl (2×10⁵ cells/well, anti-CD28 final concentration 2 μg/ml). The plates were then incubated at 37° C. for 72 hours.

125 μl supernatant was then removed from each well and stored at −20° C. until tested by ELISA for IL-2, IL-10, IFNg and IL-13using antibody pairs from R & D Systems (Abingdon, UK).

### EXAMPLE 3

### Luciferase Assay for Detecting Notch Signalling Activity

hDelta1-IgG4Fc fusion protein (Example 1) was immobilised on Streptavidin-Dynabeads (CELLection Biotin Binder Dynabeads [Cat. No. 115.21] at 4.0×10⁸ beads/ml from Dynal (UK) Ltd; “beads”) in combination with biotinylated α-IgG-4 (clone JDC14 at 0.5 mg/ml from Pharmingen [Cat. No. 555879]) as follows:

1×10⁷ beads (25 μl of beads at 4.0×10⁸ beads/ml) and 2 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG-4 in a sterile Eppendorf tube and placed on a shaker at room temperature for 30 minutes. PBS twas added to 1 ml and the mixture was spun down at 13,000 rpm for 1 minute and then washed twice more with 1 ml of PBS.

The mixture was then spun down at 13,000 rpm for 1 minute and the beads were resupsended in 50 μl PBS per sample. 50 μl of biotinylated α-IgG-4-coated beads were added to each sample and the mixture was incubated on a rotary shaker at 4 ° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended in a final volume of 100 μl of DMEM plus 10% (HI)FCS plus glutamine plus P/S, i.e. at 1.0×10⁵ beads/μl.

N27#11 cells (CHO cells expressing full length human Notch2 and a CBF1-luciferase reporter construct; T₈₀ flask; as described in WO 03/012441, Lorantis, e.g. see Example 7 therein) were removed using 0.02% EDTA solution (Sigma), spun down and resuspended in 10 ml DMEM plus 10% (HI)FCS plus glutamine plus P/S. 10 μl of cells were counted and the cell density was adjusted to 2.0×10⁵ cells/ml with fresh DMEM plus 10% (HI)FCS plus glutamine plus P/S. 100 μl per well was added to a 96-well tissue culture plate (flat bottom), i.e. 2.0×10⁴ transfected cells per well, using a multi-channel pipette and the plate was then incubated overnight.

Supernatant was then removed from all the wells, 100 μl of SteadyGlo™ luciferase assay reagent (Promega) was added and the resulting mixture left at room temperature for 5 minutes.

The mixture was then pipetted up and down 2 times to ensure cell lysis and the contents from each well were transferred to a 96 well plate (with V-shaped wells) and spun in a plate holder for 5 minutes at 1000 rpm at room temperature.

175 μl of cleared supernatant was then transferred to a white 96-well plate (Nunc) leaving the beads pellet behind.

Luminescence was then read in a TopCount™ (Packard) counter.

### EXAMPLE 4

### Reporter Assay Using Jurkat Cell Line

As Jurkat cells cannot be cloned by simple limiting dilution a methylcellulose-containing medium (ClonaCell™ TCS) was used with these cells.

Jurkat E6.1 cells (lymphoblast cell line; ATCC No TIB-152) were cloned using ClonaCell™ Transfected Cell Selection (TCS) medium (StemCell Technologies, Vancouver, Canada and Meylan, France) according to the manufacturer's guidelines.

Plasmid pLOR92 (prepared as described above) was electroporated into the Jurkat E6.1 cells with a Biorad Gene Pulser II electroporator as follows:

Actively dividing cells were spun down and resuspended in ice-cold RPMI medium containing 10% heat-inactivated FCS plus glutamine plus penicillin/streptomycin (complete RPMI) at 2.0×10⁷ cells per ml. After 10 min on ice, 0.5 ml of cells (i.e. 1×10⁷ cells) was placed into a pre-cooled 4 mm electroporation cuvette containing 20 μg of plasmid DNA (Endo-free Maxiprep DNA dissolved in sterile water). The cells were electroporated at 300 v and 950 μF and then quickly removed into 0.5 ml of warmed complete RPMI medium in an Eppendorf tube. The cells were spun for at 3000 rpm for 1 min in a microfuge and placed at 37° C. for 15 min to recover from being electroporated. The supernatant was then removed and the cells were plated out into a well of a 6-well dish in 4 ml of complete RPMI and left at 37° C. for 48 h to allow for expression of the antibiotic resistance marker.

After 48 h the cells were spun down and resupended into 10 ml fresh complete RPMI. This was then divided into 10×15 ml Falcon tubes and 8 ml of pre-warmed ClonaCell-TCS medium was added followed by 1 ml of a 10× final concentration of the antibiotic being used for selection. For G418 selection the final concentration of G418 was 1 mg/ml so a 10 mg/ml solution in RPMI was prepared and 1 ml of this was added to each tube. The tubes were mixed well by inversion and allowed to settle for 15 min at room temperature before being plated out into 10 cm tissue culture dishes. These were then placed in a CO2 incubator for 14 days when that were examined for visible colonies.

Macroscopically visible colonies were picked off the plates and these colonies were expanded through 96-well plates to 24-well plates to T25 flasks—in complete RPMI containing 1 mg/ml G418.

The resulting clones were each transiently transfected with pLOR91 using Lipofectamine 2000 reagent (according to manufacturer's protocol) and then plated out onto a 96-well plate containing plate-bound immobilised hDelta1-IgG4Fc. A well-performing clone (#24) was selected and used for luciferase assays.

### EXAMPLE 5

B1/6 mice (8 weeks, 5 days to 10 weeks, 5 days old) were grouped and treated as follows:

i) 15 mice received a) 100 ug mMOG 35-55 peptide (peptide consisting of amino acids 35-55 of Mus Musculus MOG having sequence: MEVGWYRSPFSRVVHLYRNGK; SEQ ID NO:99) with Complete Freund's Adjuvant (CFA) in phosphate buffered saline (PBS) 100 ul administered by subcutaneous (s.c.) injection plus b) 200 ng Pertussis toxin (Sigma) in PBS 100 ul administered by intraperitoneal (i.p.) injection.

ii) 5 mice were left untreated as controls.

Approximately three days later the mice from (i) received a further 200 ng Pertussis toxin in PBS 100 ul i.p

Approximately three months later (after onset of autoimmune encephalitis disease symptoms in teated mice) spleens were removed and selected spleens were passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed. The cells were incubated for 4 minutes with 5 ml ACK lysis buffer (0.15M NH₄Cl, 1.0M KHC0₃, 0.1 mM Na₂EDTA in double distilled water) per spleen (to lyse red blood cells). The cells were then washed once with R10F medium and counted.

Cells were resuspended in R10F medium at 12.5×10⁶cells/ml. 200 μl of cell suspension was added to wells of a 48-well plate to give 2.5×10⁶ cells/well.

mMOG 35-55 peptide stocks at 100 mg/ml were used for activation. 22 mls at 80 μg/ml in medium was used to give a final well concentration of 40 ug/ml or 10 ug/ml.

Human Notch ligand protein (human Delta1 extracellular domain domain fused with IgG4Fc) was coated onto Streptavidin-Dynabeads (CELLection Biotin Binder Dynabeads [Cat. No. 115.21] from Dynal (UK) Ltd) in combination with biotinylated α-IgG-4 (clone JDC14 at 0.5 mg/ml from Pharmingen [Cat. No. 555879]) to give Delta beads. Beads were added to wells at a ratio of 2 beads:1 cell, giving 5×10⁶ beads/well. Wells were mixed and plates were incubated 37° C. for 4 days before collecting supernatants for ELISA using anti-IL-10 antibody from R & D Systems.

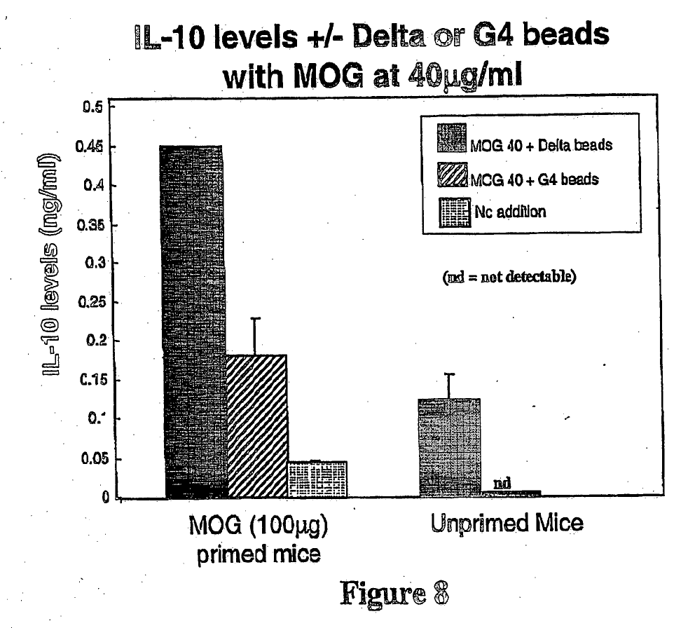

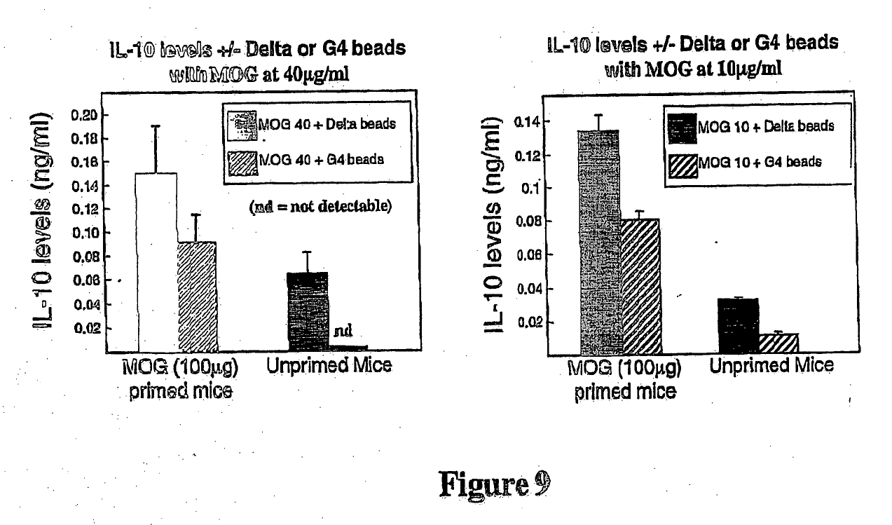

ELISA Results in FIGS. 8 and 9, show how activity of autoantigen-specific T-cells can be modified according to the present invention.

### EXAMPLE 6

i) Preparation of Notch Ligand Extracellular Domain Fragment (Activator of Notch Signalling) with Free Cysteine Tail for Particle Coupling

A protein fragment comprising amino acids 1 to 332 of human Delta 1 (DLL-1; for sequence see GenBank Accession No AF003522) and ending with a free cysteine residue (“D1E3cys”) was prepared as follows:

A template containing the entire coding sequence for the extracellular (EC) domain of human DLL-1 (with two silent mutations) was prepared by a PCR cloning strategy from a placental cDNA library made from placental polyA+ RNA (Clontech; cat no 6518-1) and combined with a C-terminal V5HIS tag in a pCDNA3.1 plasmid (Invitrogen, UK) The template was cut HindIII to PmeI to provide a fragment coding for the EC domain and this was used as a template for PCR using primers as follows:

(SEQ ID NO:100)

5′-primer: CAC CAT GGG CAG TCG GTG CGC GCT GG

(SEQ ID NO:101)

3′-primer: GTC TAC GTT TAA ACT TAA CAC TCG TCA ATC
CCC AGC TCG CAG GTG

PCR was carried out using Pfu turbo polymerase (Stratagene, La Jolla, Calif., US) with cycling conditions as follows: 95 C 5 min, 95 C 1 min, 45-69 C 1 min, 72 C 1 min for 25 cycles, 72 C 10 min.

The products at 58 C, 62 C & 67 C were purified from 1% agarose gel in 1×TAE using a Qiagen gel extraction kit according to the manufacturer's instructions, ligated into pCRIIblunt vector (InVitrogen TOPO-blunt kit) and then transformed into TOP10 cells (InVitrogen). The resulting clone sequence was verified, and only the original two silent mutations were found to be present in the parental clone.

The resulting sequence coding for “D1E3Cys” was excised using PmeI and HindIII, purified on 1% agarose gel, 1×TAE using a Qiagen gel extraction kit and ligated into pCDNA3.1V5HIS (Invitrogen) between the PmeI and HindIII sites, thereby eliminating the V5HIS sequence. The resulting DNA was transformed into TOP10 cells. The resulting clone sequence was verified at the 3′-ligation site.

The D1E3Cys-coding fragment was excised from the pCDNA3.1 plasmid using PmeI and HindIII. A pEE14.4 vector plasmid (Lonza Biologics, UK) was then restricted using EcoRI, and the 5′-overhangs were filled in using Klenow fragment polymerase. The vector DNA was cleaned on a Qiagen PCR purification column, restricted using HindIII, then treated with Shrimp Alkaline Phosphatase (Roche). The pEE14.4 vector and D1E3cys fragments were purified on 1% agarose gel in 1×TAE using a Qiagen gel extraction kit prior to ligation (T4 ligase) to give plasmid pEE14.4 DLLΔ4-8cys. The resulting clone sequence was verified.

The D1E3Cys coding sequence (SEQ ID NO:102) is as follows:

| | |
|---|---|
| 1 | atgggcagtc ggtgcgcgct ggccctggcg gtgctctcgg ccttgctgtg |
| 51 | tcaggtctgg agctctgggg tgttcgaact gaagctgcag gagttcgtca 101 |
| | acaagaaggg gctgctgggg aaccgcaact gctgccgcgg gggcgcgggg 151 |
| | ccaccgccgt gcgcctgccg gaccttcttc cgcgtgtgcc tcaagcacta 201 |
| | ccaggccagc gtgtcccccg agccgccctg cacctacggc agcgccgtca 251 |
| | cccccgtgct gggcgtcgac tccttcagtc tgcccgacgg cgggggcgcc 301 |
| | gactccgcgt tcagcaaccc catccgcttc cccttcggct tcacctggcc 351 |
| | gggcaccttc tctctgatta ttgaagctct ccacacagat tctcctgatg 401 |
| | acctcgcaac agaaaaccca gaaagactca tcagccgcct ggccacccag 451 |
| | aggcacctga cggtgggcga ggagtggtcc caggacctgc acagcagcgg 501 |
| | ccgcacggac ctcaagtact cctaccgctt cgtgtgtgac gaacactact 551 |
| | acggagaggg ctgctccgtt ttctgccgtc cccgggacga tgccttcggc 601 |
| | cacttcacct gtggggagcg tggggagaaa gtgtgcaacc ctggctggaa 651 |
| | agggccctac tgcacagagc cgatctgcct gcctggatgt gatgagcagc 701 |
| | atggattttg tgacaaacca ggggaatgca agtgcagagt gggctggcag 751 |
| | ggccggtact gtgacgagtg tatccgctat ccaggctgtc tccatggcac 801 |
| | ctgccagcag ccctggcagt gcaactgcca ggaaggctgg gggggccttt 851 |
| | tctgcaacca ggacctgaac tactgcacac accataagcc ctgcaagaat 901 |
| | ggagccacct gcaccaacac gggccagggg agctacactt gctcttgccg 951 |
| | gcctgggtac acaggtgcca cctgcgagct ggggattgac gagtgttaa |

The DNA was prepared for stable cell line transfection/selection in a Lonza GS system using a Qiagen endofree maxi-prep kit.
ii) Expression of D1E3Cys
Linearisation of DNA

The pEE14.4 DLLΔ4-8cys plasmid DNA from (i) above was linearised by restriction enzyme digestion with PvuI, and then cleaned up using phenol chloroform isoamyl alcohol (IAA), followed by ethanol precipitation. Plasmid DNA was checked on an agarose gel for linearisation, and spec'd at 260/280 nm for quantity and quality of prep.

Transfection

CHO-K1 cells were seeded into 6 wells at 7.5×10⁵ cells per well in 3 ml media (DMEM 10% FCS) 24 hrs prior to transfection, giving 95% confluency on the day of transfection.

Lipofectamine 2000 was used to transfect the cells using 5 ug of linearised DNA. The transfection mix was left on the cell sheet for 5½ hours before replacing with 3 ml semi-selective media (DMEM, 10% dFCS, GS) for overnight incubation.

At 24 hours post-transfection the media was changed to full selective media (DMEM (Dulbecco's Modified Eagle Medium), 10% dFCS (fetal calf serum), GS (glutamine synthase), 25 uM L-MSX (methionine sulphoximine)) and incubated further.

Cells were plated into 96 wells at 10⁵ cells per well on days 4 and 15 after transfection.

96 well plates were screened under a microscope for growth 2 weeks post clonal plating. Single colonies were identified and scored for % confluency. When colony size was >30% media was removed and screened for expression by dot blot against anti-human-Delta-1 antisera. High positives were confirmed by the presence of a 36 kDa band reactive to anti-human-Delta-1 antisera in PAGE Western blot of media.

Cells were expanded by passaging from 96 well to 6 well to T25 flask before freezing.

The fastest growing positive clone (LC09 0001) was expanded for protein expression.

D1E3Cys Expression and Purification

T500 flasks were seeded with 1×10⁷ cells in 80 ml of selective media. After 4 days incubation the media was removed, cell sheet rinsed with DPBS and 150 ml of 325 media with GS supplement added to each flask. Flasks were incubated for 7 further days before harvesting. Harvest media was filtered through a 0.65-0.45 um filter to clarify prior to freezing. Frozen harvests were purified by FPLC as follows:

Frozen harvest was thawed and filtered. A 17 ml Q Sepharose column was equilibrated in 0.1M Tris pH8 buffer, for 10 column volumes. The harvest was loaded onto the column using a P1 pump set at 3 ml/min, the flowthrough was collected into a separate container (this is a reverse purification—a lot of the BSA contaminant binds to the Q Sepharose FF and our target protein does not and hence remains in the flowthrough). The flowthrough was concentrated in a TFF rig using a 10 kDa cut off filter cartridge, during concentration it was washed 3× with 0.1M Sodium phosphate pH 7 buffer. The 500 ml was concentrated down to 35 ml, to a final concentration of 3 mg/ml.

Samples were run on SDS PAGE reduced and non-reduced. The amino acid sequence (SEQ ID NO: 103) of the resulting expressed D1E3Cys protein was as follows:

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDEC

(wherein the sequence in italics is the leader peptide, the underlined sequence is the DSL domain, the bold sequences are the three EGF repeats, and the terminal Cys residue is shown bold underlined).

iii) Reduction of D1E3cys Protein

40 μg D1E3Cys protein from (ii) above was made up to 100 l to include 100 mM sodium phosphate pH 7.0 and 5 mM EDTA. 2 volumes of immobilised TCEP (tris[2-carboxyethyl]phosphine hydrochloride; Pierce, Rockford, Ill., US, Cat No: 77712; previously washed 3 times 1 ml 100 mM sodium phosphate pH 7.0) were added and the mixture was incubated for 30 minutes at room temperature, with rotating.

The resin was pelleted at room temperature in a microfuge (13,000 revs/min, 5 minutes) and the supernatant was transferred to a clean Eppendorf tube and stored on ice. Protein concentration was measured by Warburg-Christian method.

iv) Purification of Expressed D1E3Cys by HIC

D1E3Cys Harvests were purified using Hydrophobic Interaction Chromatography (HIC), the eluate was then concentrated and buffer exchanged using centrifugal concentrators according to the manufacturers' instructions. The purity of the product was determined by SDS PAGE.

v) Partial Reduction of D1E3cys

D1E3cys protein (purified as in (i) above) at 1 mg/ml in 100 mM sodium phosphate pH7.0 was reduced using TCEP.HCl (Tris(2-carboxyethyl)phosphine hydrochloride; Pierce, 20490) at a 10-fold molar excess of reducing agent for 1 h at 22° C. The protein was purified by buffer exchange using Sephadex G-25, PD-10 columns (Amersham Biosciences, 17-0851-01) into 100 mM sodium phosphate pH7.0 followed by concentration in Vivaspin 6ml concentrators. Protein concentration was estimated using the Warburg-Christian A280/A260 method.

The efficiency of reduction can be estimated using the Ellman's assay. The supplied D1E3cys protein has no free thiol groups, whereas partially reduced D1E3cys is predicted to have a single free thiol group per mole of protein. Using a 96-well microtitre plate, aliqouts of D1E3cys protein or L-cysteine hydrochloride (Sigma, C-1276) were made to 196 ul in 100 mM sodium phosphate pH7.0 and 4 ul 4 mg/ml Ellman's reagent (in 100 mM sodium phosphate pH 7.0) was added. Reactions were incubated for 15 min at 22° C. and absorbance was recorded at 405 nm.

vi) Coupling of Reduced D1E3cys to Beads.

D1E3Cys was coupled to beads from Miltenyi Biotec (Bisley, Surrey, UK and Auburn, Calif., US; e.g. product reference 130-048-001) by reductive coupling. The beads are super-paramagnetic iron-dextran particles with a mean particle diameter of approximately 50 nm.

### EXAMPLE 7

### Co-Administration of KLH Beads and D1E3Cys-Coupled Microbeads In Vivo

i) Coating of Beads with KLH

Imject® Mariculture Keyhole Limpet Hemocyanin (mcKLH) in PBS Buffer (lyophilized from PBS) 20 mg (Pierce product number 77600) was reconstituted with 2.0 ml dH₂O to make a 10 mg/ml solution containing PBS, pH 7.2 with proprietary stabilizer.

Surfactant-free White Aldehyde/Sulfate Latex Beads (Interfacial Dynamics corp Portland or USA batch number 1813) concentration 5.8×10⁸ beads/ml were washed in PBS×3 (spun for 10 mins at 13 k RT). The beads were then resuspended at 2×10⁸ beads/ml in 500 μg/ml mcKLH in PBS and horizontally rotated at 37° C. overnight. Beads were then washed again in PBS×3 (spun for 10 mins at 13 k RT) and resuspended in PBS at the required concentration. Successful coating of the beads was checked by their ability to neutralize an anti-KLH antiserum in an ELISA system.

ii) In Vivo Administration with D1E3Cys-Coupled Beads

6-8 weeks old female Balb/c mice were injected s.c. at the base of the tail with 2×10⁶ KLH coated beads (prepared as described above) per mouse. Particles bearing modulators of Notch signalling (D1E3cys-coupled beads prepared as above; 0.6 or 7 μg protein per mouse); D1E3Cys protein alone (7 μg per mouse); Protein G-coupled beads (Miltenyi Cat No 130-071-101; control); or LPS 0.76 ng/mouse in Na₂PO₄ buffer (100 ul) were injected s.c. in a close separate site of the tail base (all agents were administered as aqueous solutions;100 mM sodium phosphate at pH 7). In each case 8 mice were used in each group and one group was left untreated.

Groups were thus as follows:

Untreated:

(8 mice) Untreated

KLH Only:

(8 mice), 2×10⁶ KLH beads/mouse, 100 ul s.c. at tail base+Saline 100 ul (1 site 100 ul each) s.c. tail base

KLH Plus Buffer Control:

(8 mice), 2×10⁶ KLH beads/mouse, 100 ul s.c. at tail base+LPS 0.76 ng/mouse in Na2PO4 buffer 100 ul s.c. at tail base

KLH+D1E3Cys-Beads:

(8 mice), 2×10⁶ KLH beads/mouse, 100 ul s.c. at tail base, +7 ug D1E3cys-coated Miltenyi beads/mouse, 100 ul s.c. tail base

After 14 days, mice were injected s.c. in a close separate site of the tail base with KLH 5 ng+ovalbumin (OVA) 100 ug/100 ul Saline:CFA (1:1).

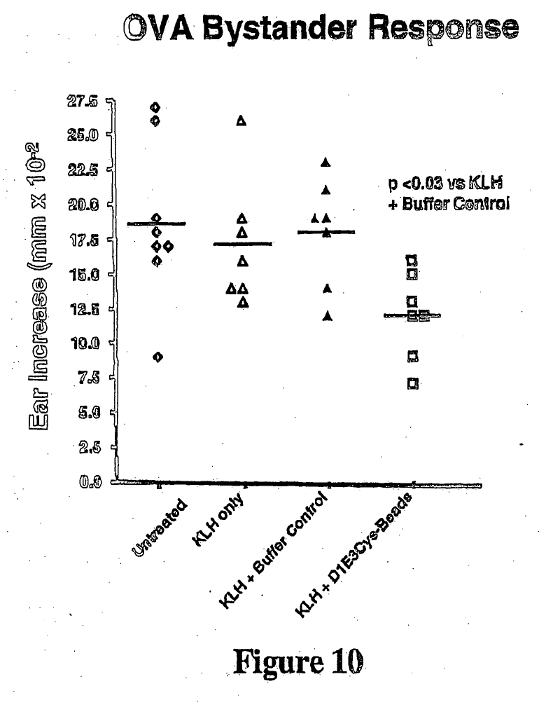

2 weeks later mice were challenged in the right ear with OVA 20 ug/20 ul. The increase in ear swelling (right ear-left ear) was measured for the following four days using a digital calliper. Results are shown in FIG. 10.

In this case KLH can be seen as the bystander antigen and OVA as the target antigen. The suppression seen in the mice treated with the D1E3Cys coated beads (modulator of Notch signaling) is indicative of a bystander suppression effect (p <0.03 vs KLH+ buffer, student's t-test).

The invention is further described by the following numbered paragraphs:

1. A product comprising i) a modulator of the Notch signalling pathway; and ii) an autoantigen or bystander antigen or antigenic determinant thereof, or a polynucleotide coding for an autoantigen or bystander antigen or antigenic determinant thereof; as a combined preparation for simultaneous, contemporaneous, separate or sequential use for modulation of immune response.

2. A product as described in paragraph 1 for modulation of peripheral T-cell activation.

3. A product as described in paragraph 1 for use in reducing an immune response to an autoantigen or bystander antigen.

4. A product as described in paragraph 1 for use in promoting immune tolerance to an autoantigen or bystander antigen.

5. A product as described in paragraph 1 for use in the treatment of autoimmune disease.

6. A product as described in any of paragraphs 1 to 5 wherein the autoantigen or bystander antigen is a nervous system autoantigen or bystander antigen.

7. A product as described in paragraph 6 wherein the autoantigen or bystander antigen is a Multiple Sclerosis autoantigen or bystander antigen.

8. A product as described in paragraph 6 wherein the autoantigen or bystander antigen is a Myasthenia Gravis autoantigen or bystander antigen.

9. A product as described in any of paragraphs 1 to 5 wherein the autoantigen or bystander antigen is a skin autoantigen or bystander antigen.

10. A product as described in paragraph 9 wherein the autoantigen or bystander antigen is a Pemphigus autoantigen or bystander antigen.

11. A product as described in any of paragraphs 1 to 5 wherein the autoantigen or bystander antigen is an endocrine autoantigen or bystander antigen.

12. A product as described in paragraph 11 wherein the autoantigen or bystander antigen is an adrenal autoantigen or bystander antigen.

13. A product as described in paragraph 11 wherein the autoantigen or bystander antigen is a thyroid autoantigen or bystander antigen.

14. A product as described in any of paragraphs 1 to 5 wherein the autoantigen or bystander antigen is a Goodpasture's autoantigen or bystander antigen.

15. A product as described in any of paragraphs 1 to 5 wherein the autoantigen or bystander antigen is a renal autoantigen or bystander antigen.

16. A product as described in any of paragraphs 1 to 5 wherein the autoantigen or bystander antigen is a Wegener's autoantigen or bystander antigen.

17. A product as described in any of paragraphs 1 to 5 wherein the autoantigen or bystander antigen is an autoimmune anemia autoantigen or bystander antigen.

18. A product as described in any of paragraphs 1 to 5 wherein the autoantigen or bystander antigen is an autoimmune thrombocytopenia autoantigen or bystander antigen.

19. A product as described in any of paragraphs 1 to 5 wherein the autoantigen or bystander antigen is an autoimmune gastritis autoantigen or bystander antigen.

20. A product as described in any of paragraphs 1 to 5 wherein the autoantigen or bystander antigen is an autoimmune hepatitis autoantigen or bystander antigen.

21. A product as described in any of paragraphs 1 to 5 wherein the autoantigen or bystander antigen is an autoimmune vasculitis autoantigen or bystander antigen.

22. A product as described in any of paragraphs 1 to 5 wherein the autoantigen or bystander antigen is an ocular autoantigen or bystander antigen.

23. A product as described in any of paragraphs 1 to 5 wherein the autoantigen or bystander antigen is a cardiac autoantigen or bystander antigen.

24. A product as described in any of paragraphs 1 to 5 wherein the autoantigen or bystander antigen is a scleroderma or myositis autoantigen or bystander antigen.

25. A product as described in any of paragraphs 1 to 5 wherein the autoantigen or bystander antigen is an autoimmune arthritis autoantigen or bystander antigen.

26. A product as described in any of paragraphs 1 to 5 wherein the autoantigen or bystander antigen is a Systemic Lupus Erythematosus (SLE) autoantigen or bystander antigen.

27. A product as described in any of paragraphs 1 to 5 wherein the autoantigen or bystander antigen is a bowel autoantigen or bystander antigen.

28. A product as described in any of paragraphs 1 to 5 wherein the autoantigen or bystander antigen is a Sjogren's autoantigen or bystander antigen.

29. A product as described in any one of the preceding paragraphs wherein the modulator of the Notch signalling pathway comprises a Notch ligand or a fragment, derivative, homologue, analogue or allelic variant thereof or a polynucleotide coding for a Notch ligand or a fragment, derivative, homologue, analogue or allelic variant thereof.

30. A product as described in any one of paragraphs 1 to 28 wherein the modulator of the Notch signalling pathway comprises a Delta or Serrate/Jagged protein or a fragment, derivative, homologue, analogue or allelic variant thereof or a polynucleotide coding for a Delta or Serrate/Jagged protein or a fragment, derivative, homologue, analogue or allelic variant thereof.

31. A product as described in paragraph 30 wherein the modulator of the Notch signalling pathway comprises a fusion protein comprising a segment of a Notch ligand extracellular domain and an immunoglobulin F_{c} segment, or a polynucleotide coding for such a fusion protein.

32. A product as described in any one of paragraphs 1 to 28 wherein the modulator of the Notch signalling pathway comprises a protein or polypeptide comprising a DSL or EGF-like domain or a polynucleotide sequence coding for such a protein or polypeptide.

33. A product as described in paragraph 32 wherein the modulator of the Notch signalling pathway comprises or codes for a Notch ligand DSL domain and at least 2 to 8 EGF-like domains.

34. A product as described in any one of paragraphs 1 to 28 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

35. A product as described in any one of paragraphs 1 to 28 wherein the modulator of the Notch signalling pathway comprises a dominant negative version of a Notch signalling repressor, or a polynucleotide which codes for a dominant negative version of a Notch signalling repressor.

36. A product as described in any one of the preceding paragraphs in the form of a pharmaceutical composition.

37. A combination of i) a modulator of the Notch signalling pathway and ii) an autoantigen or bystander antigen or antigenic determinant thereof, or a polynucleotide coding for an autoantigen or bystander antigen or antigenic determinant thereof; for simultaneous, contemporaneous, separate or sequential use for the treatment of autoimmune disease.

38. A pharmaceutical composition comprising i) a modulator of the Notch signalling pathway, ii) an autoantigen or bystander antigen or antigenic determinant thereof, or a polynucleotide coding for an autoantigen or bystander antigen or antigenic determinant thereof and iii) a pharmaceutically acceptable carrier.

39. A method for treating autoimmune disease in a mammal comprising simultaneously, contemporaneously, separately or sequentially administering, in either order:

i) an effective amount of a modulator of the Notch signalling pathway; and

ii) an effective amount of an autoantigen or bystander antigen or antigenic determinant thereof, or a polynucleotide coding for an autoantigen or bystander antigen or antigenic determinant thereof.

40. A method for reducing an immune response to an autoantigen or bystander antigen in a mammal comprising simultaneously, contemporaneously, separately or sequentially administering, in either order:

i) an effective amount of a modulator of the Notch signalling pathway; and

ii) an effective amount of an autoantigen or bystander antigen or antigenic determinant thereof, or a polynucleotide coding for an autoantigen or bystander antigen or antigenic determinant thereof.

41. A method for promoting immune tolerance to an autoantigen or bystander antigen in a mammal comprising simultaneously, contemporaneously, separately or sequentially administering, in either order:

i) an effective amount of a modulator of the Notch signalling pathway; and

ii) an effective amount of an autoantigen or bystander antigen or antigenic determinant thereof, or a polynucleotide coding for an autoantigen or bystander antigen or antigenic determinant thereof.

42. A method for producing a lymphocyte or antigen presenting cell (APC) capable of promoting tolerance to an autoantigen or bystander antigen which method comprises incubating a lymphocyte or APC obtained from a human or animal patient with (i) a modulator of the Notch signalling pathway and (ii) an autoantigen or bystander antigen or antigenic determinant thereof or a polynucleotide coding for an autoantigen or bystander antigen or antigenic determinant thereof, in either order.

43. A method according to paragraph 42 which comprises incubating a lymphocyte or APC obtained from a human or animal patient with an APC in the presence of (i) a modulator of the Notch signalling pathway and (ii) an autoantigen or bystander antigen or antigenic determinant thereof or a polynucleotide coding for an autoantigen or bystander antigen or antigenic determinant thereof, in either order.

44. A method according to paragraph 42 for producing an APC capable of promoting tolerance to an autoantigen or bystander antigen in a T cell which method comprises contacting an APC with (i) a modulator of the Notch signalling pathway and (ii) an autoantigen or bystander antigen or antigenic determinant thereof or a polynucleotide coding for an autoantigen or bystander antigen or antigenic determinant thereof, in either order.

45. A method for producing a T cell capable of promoting tolerance to an autoantigen or bystander antigen which method comprises incubating an antigen presenting cell (APC) simultaneously or sequentially, in any order, with:

(i) an autoantigen or bystander antigen or antigenic determinant thereof or a polynucleotide coding for an autoantigen or bystander antigen or antigenic determinant thereof;

(ii) a modulator of the Notch signalling pathway; and

(iii) a T cell obtained from a human or animal patient.

46. A method for producing a lymphocyte or APC capable of promoting tolerance to an autoantigen or bystander antigen which method comprises incubating a lymphocyte or APC obtained from a human or animal patient with a lymphocyte or APC produced by the method of any one of paragraphs 42 to 45.

47. A method as described in any one of paragraphs 42 to 46 wherein the lymphocyte or APC is incubated ex-vivo.

48. A method for promoting tolerance to an autoantigen or bystander antigen which method comprises administering to the patient an APC or lymphocyte produced by the method of any one of paragraphs 42 to 47.

49. A method as described in any one of paragraphs 42 to 48 wherein the modulator of the Notch signalling pathway comprises a Notch ligand or a fragment, derivative, homologue, analogue or allelic variant thereof or a polynucleotide coding for a Notch ligand or a fragment, derivative, homologue, analogue or allelic variant thereof.

50. A method as described in paragraph 49 wherein the modulator of the Notch signalling pathway comprises a Delta or Serrate/Jagged protein or a fragment, derivative, homologue, analogue or allelic variant thereof or a polynucleotide coding for a Delta or Serrate/Jagged protein or a fragment, derivative, homologue, analogue or allelic variant thereof.

51. A method as described in any one of paragraphs 42 to 50 wherein the modulator of the Notch signalling pathway comprises a fusion protein comprising a segment of a Notch ligand extracellular domain and an immunoglobulin F_{c} segment, or a polynucleotide coding for such a fusion protein.

52. A method as described in any one of paragraphs 42 to 50 wherein the modulator of the Notch signalling pathway comprises a protein or polypeptide comprising at least one Notch ligand DSL domain and at least one EGF-like domain or a polynucleotide sequence coding for such a protein or polypeptide.

53. A method as described in any one of paragraphs 42 to 48 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

54. A method as described in any one of paragraphs 42 to 48 wherein the modulator of the Notch signalling pathway comprises a dominant negative version of a Notch signalling repressor, or a polynucleotide which codes for a dominant negative version of a Notch signalling repressor.

55. A modulator of the Notch signalling pathway for use to treat autoimmune disease in simultaneous, contemporaneous, separate or sequential combination with an autoantigen or bystander antigen or antigenic determinant thereof or a polynucleotide coding for an autoantigen or bystander antigen or antigenic determinant thereof.

56. The use of a combination of i) a modulator of the Notch signalling pathway; and ii) an autoantigen or bystander antigen or antigenic determinant thereof, or a polynucleotide coding for an autoantigen or bystander antigen or antigenic determinant thereof, in the manufacture of a medicament for the treatment of autoimmune disease.

57. The use of a modulator of the Notch signalling pathway in the manufacture of a medicament for treatment of autoimmune disease in simultaneous, contemporaneous, separate or sequential combination with an autoantigen or bystander antigen or antigenic determinant thereof, or a polynucleotide coding for an autoantigen or bystander antigen or antigenic determinant thereof.

58. A conjugate comprising first and second sequences, wherein the first sequence comprises an autoantigen or bystander antigen or a polynucleotide sequence coding for an autoantigen or bystander antigen or antigenic determinant thereof, and the second sequence comprises a polypeptide or polynucleotide for Notch signalling modulation.

59. A conjugate as described in paragraph 58 in the form of a vector comprising a first polynucleotide sequence coding for a modulator of the Notch signalling pathway and a second polynucleotide sequence coding for an autoantigen or bystander antigen or antigenic determinant thereof.

60. A conjugate as described in paragraph 59 in the form of an expression vector.

61. A conjugate as described in any one of paragraphs 58 to 60 wherein the first polynucleotide sequence codes for a Notch ligand or a fragment, derivative, homologue, analogue or allelic variant thereof.

62. A conjugate as described in paragraph 61 wherein the first polynucleotide sequence codes for a Delta or Serrate/Jagged protein or a fragment, derivative, homologue, analogue or allelic variant thereof.

63. A conjugate as described in any one of paragraphs 58 to 62 wherein the first polynucleotide sequence codes for a protein or polypeptide comprising at least one DSL domain and at least one EGF-like domain or a fragment, derivative, homologue, analogue or allelic variant thereof.

64. A conjugate as described in paragraph 63 wherein the first polynucleotide sequence codes for a protein or polypeptide comprising at least one Notch ligand DSL domain and at least 3 to 8 EGF-like domains.

65. A conjugate as described in any one of paragraphs 58 to 60 wherein the first polynucleotide sequence codes for Notch intracellular domain (Notch IC) or a fragment, derivative, homologue, analogue or allelic variant thereof.

66. A conjugate as described in any one of paragraphs 58 to 60 wherein the first polynucleotide sequence codes for a dominant negative version of a Notch signalling repressor.

67. A conjugate as described in any one of paragraphs 58 to 66 wherein the first or second sequences are operably linked to one or more promoters.

68. A pharmaceutical or veterinary kit comprising a modulator of the Notch signalling pathway and an autoantigen or bystander antigen or antigenic determinant thereof, or a polynucleotide coding for an autoantigen or bystander antigen or antigenic determinant thereof.

69. A method for reducing an immune response to a target disease antigen or antigenic determinant thereof by administering a bystander antigen or antigenic determinant thereof (or a polynucleotide coding for such an antigen or antigenic determinant) and simultaneously, separately or sequentially administering an activator of Notch signalling.

70. A method for reducing an immune response to a target disease autoantigen or antigenic determinant thereof, by administering a bystander antigen or antigenic determinant thereof (or a polynucleotide coding for such an antigen or antigenic determinant) and simultaneously, separately or sequentially administering an activator of Notch signalling.

71. A method as described in paragraph 69 or paragraph 70 wherein the modulator of the Notch signalling pathway comprises a Notch ligand or a fragment, derivative, homologue, analogue or allelic variant thereof or a polynucleotide coding for a Notch ligand or a fragment, derivative, homologue, analogue or allelic variant thereof.

72. A method as described in paragraph 71 wherein the modulator of the Notch signalling pathway comprises a Delta or Serrate/Jagged protein or a fragment, derivative, homologue, analogue or allelic variant thereof or a polynucleotide coding for a Delta or Serrate/Jagged protein or a fragment, derivative, homologue, analogue or allelic variant thereof.

73. A method as described in paragraph 72 wherein the modulator of the Notch signalling pathway comprises a fusion protein comprising a segment of a Notch ligand extracellular domain and an immunoglobulin F_{c} segment, or a polynucleotide coding for such a fusion protein.

74. A method as described in any one of paragraphs 71 to 73 wherein the modulator of the Notch signalling pathway comprises a protein or polypeptide comprising at least one Notch ligand DSL domain and at least one EGF-like domain or a polynucleotide sequence coding for such a protein or polypeptide.

75. A method as described in any one of paragraphs 71 to 74 wherein the modulator of Notch signalling comprises a protein or polypeptide comprising:

i) a Notch ligand DSL domain;

ii) 1-5 Notch ligand EGF domains;

iii) optionally all or part of a Notch ligand N-terminal domain; and

iv) optionally one or more heterologous amino acid sequences;

or a polynucleotide coding therefor.

76. A method as described in any one of paragraphs 71 to 74 wherein the modulator of Notch signalling comprises a protein or polypeptide comprising:

i) a Notch ligand DSL domain;

ii) 2-4 Notch ligand EGF domains;

iii) optionally all or part of a Notch ligand N-terminal domain; and

iv) optionally one or more heterologous amino acid sequences;

or a polynucleotide coding therefor.

77. A method as described in any one of paragraphs 71 to 74 wherein the modulator of Notch signalling comprises a protein or polypeptide comprising:

i) a Notch ligand DSL domain;

ii) 2-3 Notch ligand EGF domains;

iii) optionally all or part of a Notch ligand N-terminal domain; and

iv) optionally one or more heterologous amino acid sequences;

or a polynucleotide coding therefor.

78. A method as described in any one of paragraphs 71 to 74 wherein the modulator of Notch signalling comprises a protein or polypeptide with at least 50%, amino acid sequence similarity to the following sequence along the entire length of the latter:

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDEC

79. A product for reducing an immune response to a target disease antigen or antigenic determinant thereof comprising i) a bystander antigen or antigenic determinant thereof (or a polynucleotide coding for such an antigen or antigenic determinant) and ii) an activator of Notch signalling, for simultaneous, separate or sequential administration for reducing an immune response to a target disease antigen.

80. The use of an activator of Notch signaling in simultaneous, separate or sequential combination with a bystander antigen or antigenic determinant thereof (or a polynucleotide coding for such an antigen or antigenic determinant) for reducing an immune response to a target antigen.

### REFERENCES (INCORPORATED HEREIN BY REFERENCE THERETO)

Altman J D et al. Science 1996 274: 94-6.
Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Dkuz et al. (1997) Cell 90: 271-280.
Hemmati-Brivanlou and Melton (1997) Cell 88:13-17.
Hicks C, et al. (2000) Nat. Cell. Biol. 2:515-520.
Iemura et al. (1998) PNAS 95:9337-9345.
Irvine K D (1999) Curr. Opin. Genet. Devel. 9:434-441.
Ju B J, et al. (2000) Nature 405:191-195.
Leimeister C. et al. (1999) Mech Dev 85(1-2):173-7.
Li et al. (1998) Immunity 8(1):43-55.
Lieber, T. et al. (1993) Genes Dev 7(10):1949-65.
Lu, F. M. et al. (1996) Proc Natl Acad Sci 93(11):5663-7.
Matsuno K, et al. (1998) Nat. Genet. 19:74-78.
Matsuno, K. et al. (1995) Development 121(8):2633-44.
McGuinness T. et al. (1996) Genomics 35(3):473-85.
Medhzhitov et al. (1997) Nature 388:394-397.
Meuer S. et al. (2000) Rapid Cycle Real-time PCR, Springer-Verlag Berlin and Heidelberg GmbH & Co.
Moloney DJ, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Sasai et al. (1994) Cell 79:779-790.
Schroeter E H, et al. (1998) Nature 393:382-386.
Schroeter, E. H. et al. (1998) Nature 393(6683):382-6.
Struhl G, Adachi A. (1998) Cell 93:649-660.
Struhl, G. et al. (1998) Cell 93(4):649-60.
Takebayashi K. et al. (1994) J Biol Chem 269(7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.
Zhao et al. (1995) J. Immunol. 155:3904-3911.

Various modifications and variations of the described methods and system of the invention will be apparent to those skilled in the art without departing from the scope and spirit of the invention. Although the invention has been described in connection with specific preferred embodiments, it should be understood that the invention as described should not be unduly limited to such specific embodiments. Indeed, various modifications of the described modes for carrying out the invention which are obvious to those skilled in chemistry, biology or related fields are intended to be within the scope of the following claims.

1. A composition comprising i) a modulator of the Notch signalling pathway; ii) an autoantigen or bystander antigen or antigenic determinant thereof, or a polynucleotide encoding an autoantigen or bystander antigen or antigenic determinant thereof; and optionally, iii) a pharmaceutically acceptable carrier.

2. The composition as claimed in claim 1, wherein the autoantigen or bystander antigen is selected from the group consisting of a nervous system autoantigen or bystander antigen, a skin autoantigen or bystander antigen, an endocrine autoantigen or bystander antigen, a Goodpasture's autoantigen or bystander antigen, a renal autoantigen or bystander antigen, a Wegener's autoantigen or bystander antigen, an autoimmune anemia autoantigen or bystander antigen, an autoimmune thrombocytopenia autoantigen or bystander antigen, an autoimmune gastritis autoantigen or bystander antigen, an autoimmune hepatitis autoantigen or bystander antigen, an autoimmune vasculitis autoantigen or bystander antigen, an ocular autoantigen or bystander antigen, a cardiac autoantigen or bystander antigen, a scleroderma or myositis autoantigen or bystander antigen, an autoimmune arthritis autoantigen or bystander antigen, a Systemic Lupus Erythematosus (SLE) autoantigen or bystander antigen, a bowel autoantigen or bystander antigen and a a Sjogren's autoantigen or bystander antigen.

3. The composition as claimed in claim 1, wherein the modulator of the Notch signalling pathway comprises or encodes:
(i) a Notch ligand or a fragment, derivative, homologue, analogue or allelic variant thereof;
(ii) a fusion protein comprising a segment of a Notch ligand extracellular domain and an immunoglobulin F_{c} segment;
(iii) a DSL or EGF-like domain;
(iv) Notch intracellular domain (Notch IC) or a fragment, derivative, homologue, analogue or allelic variant thereof; or
(v) a dominant negative version of a Notch signalling repressor.

4. The composition as claimed in claim 3, wherein the modulator of the Notch signalling pathway comprises or encodes a Delta or Serrate/Jagged protein or a fragment, derivative, homologue, analogue or allelic variant thereof.

5. The composition as claimed in claim 3, wherein the modulator of the Notch signalling pathway comprises or encodes a Notch ligand DSL domain and at least 2 to 8 EGF-like domains.

6. A method for reducing an immune response or promoting immune tolerance to an autoantigen or bystander antigen in a mammal comprising simultaneously, contemporaneously, separately or sequentially administering to the mammal, in either order:
i) an effective amount of a modulator of the Notch signalling pathway; and
ii) an effective amount of an autoantigen or bystander antigen or antigenic determinant thereof, or a polynucleotide encoding an autoantigen or bystander antigen or antigenic determinant thereof.

7. The method as claimed in claim 6, wherein the modulator of the Notch signalling pathway comprises or encodes:
(i) a Notch ligand or a fragment, derivative, homologue, analogue or allelic variant thereof;
(ii) a fusion protein comprising a segment of a Notch ligand extracellular domain and an immunoglobulin F_{c} segment;
(iii) a DSL or EGF-like domain;
(iv) Notch intracellular domain (Notch IC) or a fragment, derivative, homologue, analogue or allelic variant thereof; or
(v) a dominant negative version of a Notch signalling repressor.

8-16. (canceled)

17. A conjugate comprising first and second sequences, wherein the first sequence comprises or encodes an autoantigen or bystander antigen or antigenic determinant thereof, and the second sequence comprises or encodes a modulator of Notch signalling.

18. A vector comprising the conjugate as claimed in claim 17, wherein the first and second sequences are polynucleotide sequences.

19. The conjugate as claimed in claim 17, wherein the first sequence is a polynucleotide sequence that encodes:
(i) a Notch ligand or a fragment, derivative, homologue, analogue or allelic variant thereof;
(ii) at least one DSL domain and at least one EGF-like domain or a fragment, derivative, homologue, analogue or allelic variant thereof;
(iii) Notch intracellular domain (Notch IC) or a fragment, derivative, homologue, analogue or allelic variant thereof; or
(iv) a dominant negative version of a Notch signalling repressor.

20. The conjugate as claimed in claim 19, wherein the first sequence encodes a Delta or Serrate/Jagged protein or a fragment, derivative, homologue, analogue or allelic variant thereof.

21. The conjugate as claimed in claim 19, wherein the first sequence encodes a protein or polypeptide comprising at least one Notch ligand DSL domain and at least 3 to 8 EGF-like domains.

22. The conjugate as claimed in claim 18 wherein the first and/or second sequences are operably linked to one or more promoters.

23. A kit comprising (i) a modulator of the Notch signalling pathway and (ii) an autoantigen or bystander antigen or antigenic determinant thereof, or a polynucleotide encoding an autoantigen or bystander antigen or antigenic determinant thereof.

24. A method for reducing an immune response in a subject to a target disease antigen or antigenic determinant thereof by administering to the subject a bystander antigen or antigenic determinant thereof or a polynucleotide encoding the antigen or antigenic determinant, and simultaneously, separately or sequentially administering an activator of Notch signalling.

25. The method as claimed in claim 24, wherein the target disease antigen is an autoantigen.

26. The method as claimed in claim 24, wherein the activator of Notch signalling comprises or encodes:
(i) a Notch ligand or a fragment, derivative, homologue, analogue or allelic variant thereof;
(ii) a fusion protein comprising a segment of a Notch ligand extracellular domain and an immunoglobulin Fc segment;
(iii) at least one DSL domain and at least one EGF-like domain;
(iv) Notch intracellular domain (Notch IC) or a fragment, derivative, homologue, analogue or allelic variant thereof; or
(v) a dominant negative version of a Notch signalling repressor.

27. The method as claimed in claim 26, wherein the activator of Notch signalling comprises or encodes a Delta or Serrate/Jagged protein or a fragment, derivative, homologue, analogue or allelic variant thereof.

28. The method as claimed in 24, wherein the activator of Notch signalling comprises a protein or polypeptide comprising:
i) a Notch ligand DSL domain;
ii) 1-5 Notch ligand EGF domains;
iii) optionally, all or part of a Notch ligand N-terminal domain; and
iv) optionally, one or more heterologous amino acid sequences;
or a polynucleotide coding therefor.

29. The method as claimed in claim 24, wherein the activator of Notch signalling comprises a protein or polypeptide with at least 50%, amino acid sequence identity to SEQ ID NO:1.

30. A composition comprising i) a bystander antigen or antigenic determinant thereof or a polynucleotide encoding the antigen or antigenic determinant and ii) an activator of Notch signalling, for simultaneous, separate or sequential administration for reducing an immune response to a target disease antigen.
